SEQUENCE LISTING (67 sequences)

SEQ ID NO: 1 - protein, 892 aa, synthetic construct
MTNYTITKETNKHSFTVPLNIAFANANAKGKGIDKLLNEAKRSKGGITEGQINAVSNVIGKKLEDIRNYFSHNYHKEFALEFESNDPVKIFLDETYEEAKKRLQIGAKSDGYKIPELPLFEKTQNGKCKITKAGAIFLASFFCHRSYVNKMLAGISGFKRDDREEFNDGQKIDYGFTKKLLSYYCLRDSYSVNTQDKHLKTFRDILGYLSRPPLEAIKWLAEKNKLEQNDRAMFFIAGQDKPQETEEDRDDKNQKFGLRKTDKFILFAVRFIEDWAETTSSKIEFARYEKIVKEKEDAKYNGKEIRVVEFPSDKQKKEEQKEIRKKQYYIRNNHAVIKITPDGKNPVSARISENELKYLVLAILDDNVQGQKAINAISSHINWIMGEINEPKSQSSYLPDFVIKEESADIKQLAEMRVKYIKEKIEKVKSQIKEEQDLQKQETGKPAEYRTWLIYKGRKISIVLKYVNNCIAEYKDRLSVEEYNKLQEYLQTNAFDKFYGILAGYEESGRLEKGVVNSIRTMKDLSGLCVKVCDMQIERIEKLKTAGGSEFYKYIGLEPKIFNQKQSEYNTKAENFLINWLSIPENFLRRKFYDAKCQKQDCLRKTPNTKKSNSQVKQRKYLIDIIKEKKFRPIHKEAFYLLGEDPRGYSRADGAIIRQHCDIYCKDALCMKMAEWYYKNRTGDFQRFIEWSPQGNAQHSYAGYKLEYTATDKIKICFEPADFTRLDIIDKPDTISNICKRYEKELLDGKLDNRINWYDFRDKALKEHRKYQGSAVADTFWFEKDLKIAEKEWQGRNETHVPFNKEEEKKGEAIKNPVNILDEAEGQGKIKSTDKEALNIVRKDCFHERCSAADEQVGIFKKLISDKAANGRKAENKAGNFQKNRRKNGWKR

SEQ ID NO: 2 - RNA, 36 nt, synthetic construct
  source 1..36: mol_type = other RNA
gctggagcagcacccgatttgcggtgtaatcacagc

SEQ ID NO: 3 - protein, 878 aa, synthetic construct
MAVNYSLREKWYRGVNKCCFTVALNIAVDNCKSKGCETLLKEAEHSKGGITDEQIQQSYTEVEKRLNDIRNYFSHFYHGDECLIFKKDDIVKRFMESVFATAVSNVVGGTKESDYKGVVPPLFEQSNEDYMITAAGVIFLASFFCHRSNVYRMLGAVKGFKHTGKEELSDGQKRDHGFTHRLLAHYSLRDSYSVKIEETKSFRDLLGYLSRVPQQAVDWLNERNELSEDEKKEFLNQKSSEEESPEQPEPENAEWRTEKTSRRSLRKTEKFILFAAKFIEDRAEKEKQDVTFARYQKTVTKEENKNQDGKQARVVRLKYEEDKKDDEKPREHFNLEWMYYIRNEHAIIQIKPKDKEAVAARISENELKYLVLLIFEGKGGDAFNKLSDYIFRMTQKIKSGQINPNEARLPSFLKNPVKNITDKMVRNRLDYIRGQIKDVLEKINMEEPQNNKWLIYKGKKISLVLKFISDGISDIKKRPNVKEYDTLRDTLQKLDFNRFYERLKSYVSDGRLAAALYDKIKGIDDISELCKKVCELMFARLAELEKKGGFELYRYIGMEVQEKDEKYDEWNSPQKKAERFLESQFSIGENFLRESFYSEYCQKQECIDKEISLNTSVKNRKSLVYIVKEKLKDIMPLHNDRWYLIDRNPKDFERKDSKVIKGLCNTYVQDVLCMKMARWYYGQLNPALKNNIKWDETGQGHGYDRYKLSYRTNFGITIEFKLADFTRLDIIEKSDMIENICRSFIKPNRTISWYDFKQDGVEEYRKRQYKAVRAVFAFEESLIIPGRDWLSQGFVPFIKNEEYVKKGFSLFVLDEAVRQLKIKGSDKDAMRQVRNDFFHEQFQAKDEQWKVFEGYLSCFMIDRPKGEKNKKRYNGNKK

SEQ ID NO: 4 - RNA, 36 nt, synthetic construct
  source 1..36: mol_type = other RNA
gctggagcagccctcgatttgcagggtaatcacagc

SEQ ID NO: 5 - protein, 871 aa, synthetic construct
MEKYLIKNFEGINKSKFTVALNIANDNCKNKGIQELLKEAQRSKGGITDTQITEVQEHIKERLNSVRNYFSHCYHEKKPLYFEANDPVKIFLEETFAKAVENLQGRFLSDKYKLTVPPLFEPNQNNTITAAGVIFLASFFCHRSYVYRMLGGIPGFKRSDKKKWGDGQKIDYGFTRKLMSFYSLRDSYSVNVQENKELTAFRDILGYLARVPGQAIDWLIEKGKLTKEEGKQFYLGEQSEEREEKAKKEEIKYALRKTDKFMLFAVRFIEDWAEQERIKVEFARYEKMTIVNENKKQDEKEERKVKFVSDEPTAAGWTYYIRNNHAIIKIIPDDKKKKAVSARISENELKYLVLTIIDGNGKNAIAYIGDYIFRTARQIENKSYNAESEKYAPAFVRGGQKKSVDKRIKYIRDEIQQVINDIEAEQEKQKNEQDAPAENRTWLIYKGKKISIILRYVNDNIAEYKKRLSVTEYNELRGYLQQLDFINFHRKLAEYQHHGRLPNGFAESINKFQDLSKLCIEVCERQKKKLQEMAAKGGIELEQYIGLAPKEENQEQNKYATKANNFIKVWLSIPENFLRQKFYDKFCKQQECKNKGSDKPDNTSVPQRKYFIAIIREKNIRPIHADKYYLLGQNPKDYERPDGKIIRQLCDVYCKDGLCMAMAKWYYENRLGKFKDLIEWQTGDDKQQHGYAGHTLEYQATEKIKIRFKLADFTRLDIIEPPERVKNICRQWETELLKKTRDGTISWYDFKLNGLEPYRQWQGYAVADIFWFEESLKINETQWQGRTHMPFNFEKDKPLWCNILDEAVKQNKIEKQDTQALRRVRHDCFHEEFLANYEQLKIFKNLISDKAKDAKPKDKKSRKNEQKYGKR

SEQ ID NO: 6 - RNA, 36 nt, synthetic construct
  source 1..36: mol_type = other RNA
gctggagcagccctcgatttgcagggttatcacagc

SEQ ID NO: 7 - protein, 798 aa, synthetic construct
MEKEQGLYSIDRYQGAGKWCFAIGANRAWDNYNERPKLFSESLLRYEKATRRDWFDEETRGLIKKSDVRQRLRKIRCYFSHYCHDNTCLGFDPDDDLRKIMEKAYERAIFEQRKHLSTETDIETPALFEPHGRITAAGVVFFCSFFVERRILNRLMGRIPGFKKTEGEYGATRQMFSKYCLRDSYSIRASDSNAVLFRDILGYLSRAPSQYYRHNKDQCDKDGHPERKKDKFINLALRYLESFVPARLRNHTLSVGRKEVVRMETNAVAEGEGEYRPYPPKAKVKVVFTEDDPERPYYITHNTVILQTAKKEEDIHHCKVGVNELKYLVLLCLQGKAEKAVAGIEGYVRRIQGRFADHTNKVARDDDERLVRGLPEFVRVASGIETPDEVRELKSRLDHIRKKWQTKKAESAEAQLHRKARDVLRHINWESQRPLGIEQYNRLLELLVNRDLESFAAEMKELKRRGLISEELLKSVEGIRNLNTLHVKVCNLVLTRLEHLVENDPEELKRHIGIVPREEKEGPSYEEKVRAFVQQPMMYRGFLRNSFFKGSGKSFAKLVEEELHKKGCPDVPLGTDYYLVRDLERDERKNRFHNDNAALYETLALDRLCVLMARDCLVRLNRNLEKHATRISWEATDAGDTICLELPRRDRDHESFRLSFGVRDYPKLYVMDDPVFLCGLMKHFFPDNQAIQYHELYSEGINKYTAMQAEGIAATLKLEEKTIKEKNMQIPATGYIRFCEIVSQSDFAPGEKRVLKNVRNGLLHYHLEFEPTEWAEFREIMKREGFDTAKKRKSTRKK

SEQ ID NO: 8 - RNA, 36 nt, synthetic construct
  source 1..36: mol_type = other RNA
gctgaagcaaccctggttttgcggggtgattacagc

SEQ ID NO: 9 - protein, 901 aa, synthetic construct
MQNYISDKQNREDSYSIDKIKGIGKWCFGLVLNKALDNVNPKDKTEIKEYEKKDLFCKSLLRFQGIKKQQWYDGTIAQDIINNKDKAAELLTNLRNYFSHHCHTDDCLNFGKESTNKEIRRIMETAYERAKEDLAGRKTGEEVSGEAEKDKDGNIKKYRLEKIPWPELFDKRDKITKAGVVFFASLFTQRSQVNRLMSWISGLKKNEDEFGMTRRALSFYSLPDSYAEEAVDLAIDESGIRKTITEQARMFRDILGYLRRIPSETYKIYHKNEEKKKYSETEKDNEEKEDKETGPVERKTDKFAFFAMKYLEDSEKVQFARYRINQETRKNEIFFDEDELKKLIEEKDVPEQEKDKRYDDYKYYFVENNAILKTDKGNIRIGINELKYFTLLSLDGKGKEAIGKIKSFLAKYTLYNLANKSFVEKYKKELPPFILTEYEILTEDEKTKTEKRLKYLKEKWINKKKEYQNLRINNKVGGILRFINENLKKDRKLDAEEYNNLQKLLTMERFGEFEKELLKFEDERESRIKKDVMKDIKGIKSIDAMFLKVCEIVLKKLEALKGDELKEYIGLNRKPLINTRAAINQKFENVLERFIQNKISLPNGMLRELYLRENNLDTGEQKDRQKFNVSVENLLKTKYTGFDVDLDKKYYEYEIDKSKRDIEGSKIKRELREILAKDRLCLMMGMKYYKDIREEFKNENGVDIRWEKENNKRVIYADILDENKSNLLTLKFGEKDYAKMYVMDRPKFIRQVWKNFVKKSGVIEYHEFYKDGIEKGLGNFQTDVIKKVLMLEEKIIEKAAGKGLTPIQINDKLFFEIKKRSRQVKNDFEKHKEVFVQNDAILKAFITSDKDIDILKKIRNAAFHYQADFQPEEKNAFDEIMKNNDIVIEEKVINKQKKWKK

SEQ ID NO: 10 - RNA, 36 nt, synthetic construct
  source 1..36: mol_type = other RNA
gctggagtagccctctatttgagtggtgattacagc

SEQ ID NO: 11 - protein, 1226 aa, synthetic construct
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLGSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 12 - protein, 701 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 13 - protein, 739 aa, Homo sapiens
MASVLGSGRGSGGLSSQLKCKSKRRRRRRSKRKDKVSILSTFLAPFKHLSPGITNTEDDDTLSTSSAEVKENRNVGNLAARPPPSGDRARGGAPGAKRKRPLEEGNGGHLCKLQLVWKKLSWSVAPKNALVQLHELRPGLQYRTVSQTGPVHAPVFAVAVEVNGLTFEGTGPTKKKAKMRAAELALRSFVQFPNACQAHLAMGGGPGPGTDFTSDQADFPDTLFQEFEPPAPRPGLAGGRPGDAALLSAAYGRRRLLCRALDLVGPTPATPAAPGERNPVVLLNRLRAGLRYVCLAEPAERRARSFVMAVSVDGRTFEGSGRSKKLARGQAAQAALQELFDIQMPGHAPGRARRTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGEGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDAEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLTL

SEQ ID NO: 14 - protein, 111 aa, synthetic construct
MAVNYSLREKWYRGVNKCCFTVALNIAVDNCKSKGCETLLKEAEHSKGGITDEQIQQSYTEVEKRLNDIRNYFSHFYHGDECLIFKKDDIVKRFMESVFATAVSNVVGGTK

SEQ ID NO: 15 - protein, 126 aa, synthetic construct
WYDFKQDGVEEYRKRQYKAVRAVFAFEESLIIPGRDWLSQGFVPFIKNEEYVKKGFSLFVLDEAVRQLKIKGSDKDAMRQVRNDFFHEQFQAKDEQWKVFEGYLSCFMIDRPKGEKNKKRYNGNKK

SEQ ID NO: 16 - protein, 6 aa, synthetic construct
RNYFSH

SEQ ID NO: 17 - protein, 6 aa, synthetic construct
RCYFSH

SEQ ID NO: 18 - protein, 6 aa, synthetic construct
RNDFFH

SEQ ID NO: 19 - protein, 6 aa, synthetic construct
RKDCFH

SEQ ID NO: 20 - protein, 6 aa, synthetic construct
RHDCFH

SEQ ID NO: 21 - protein, 6 aa, synthetic construct
RNGLLH

SEQ ID NO: 22 - protein, 6 aa, synthetic construct
RNAAFH

SEQ ID NO: 23 - protein, 430 aa, synthetic construct
KAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNVVISKPQEEKNFYLCPV

SEQ ID NO: 24 - protein, 385 aa, synthetic construct
QLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLT

SEQ ID NO: 25 - protein, 298 aa, synthetic construct
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFN

SEQ ID NO: 26 - protein, 564 aa, synthetic construct
MLRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 27 - protein, 198 aa, synthetic construct [sequence omitted]

SEQ ID NO: 28 - protein, 188 aa, synthetic construct
MDSLLMNRRKFLYQFKNVRWAKGRRETYLCYVVKRRDSATSFSLDFGYLRNKNGCHVELLFLRYISDWDLDPGRCYRVTWFTSWSPCYDCARHVADFLRGNPNLSLRIFTARLYFCEDRKAEPEGLRRLHRAGVQIAIMTFKENHERTFKAWEGLHENSVRLSRQLRRILLPLYEVDDLRDAFRTLGL

SEQ ID NO: 29 - protein, 7 aa, synthetic construct
PKKKRKV

SEQ ID NO: 30 - protein, 16 aa, synthetic construct
KRPAATKKAGQAKKKK

SEQ ID NO: 31 - protein, 9 aa, synthetic construct
PAAKRVKLD

SEQ ID NO: 32 - protein, 11 aa, synthetic construct
RQRRNELKRSP

SEQ ID NO: 33 - protein, 38 aa, synthetic construct
NQSSNFGPMKGGNFGGRSSGPYGGGGQYFAKPRNQGGY

SEQ ID NO: 34 - protein, 42 aa, synthetic construct
RMRIZFKNKGKDTAELRRRRVEVSVELRKAKKDEQILKRRNV

SEQ ID NO: 35 - protein, 8 aa, synthetic construct
VSRKRPRP

SEQ ID NO: 36 - protein, 8 aa, synthetic construct
PPKKARED

SEQ ID NO: 37 - protein, 8 aa, synthetic construct
PQPKKKPL

SEQ ID NO: 38 - protein, 12 aa, synthetic construct
SALIKKKKKMAP

SEQ ID NO: 39 - protein, 5 aa, synthetic construct
DRLRR

SEQ ID NO: 40 - protein, 7 aa, synthetic construct
PKQKKRK

SEQ ID NO: 41 - protein, 10 aa, synthetic construct
RKLKKKIKKL

SEQ ID NO: 42 - protein, 10 aa, synthetic construct
REKKKFLKRR

SEQ ID NO: 43 - protein, 20 aa, synthetic construct
KRKGDEVDGVDEVAKKKSKK

SEQ ID NO: 44 - protein, 17 aa, synthetic construct
RKCLQAGMNLEARKTKK

SEQ ID NO: 45 - protein, 30 aa, synthetic construct
MDSLLMNRRKFLYQFKNVRWAKGRRETYLC

SEQ ID NO: 46 - protein, 18 aa, synthetic construct
KRTADGSEFESPKKKRKV

SEQ ID NO: 47 - protein, 17 aa, synthetic construct
KRTADGSEFEPKKKRKV

SEQ ID NO: 48 - protein, 5 aa, synthetic construct
  REGION >5: note = the entire sequence of amino acids 1-5 can be repeated one or more times
GSGGS

SEQ ID NO: 49 - protein, 6 aa, synthetic construct
  REGION >6: note = the entire sequence of amino acids 1-6 can be repeated one or more times
GGSGGS

SEQ ID NO: 50 - protein, 4 aa, synthetic construct
  REGION >4: note = the entire sequence of amino acids 1-4 can be repeated one or more times
GGGS

SEQ ID NO: 51 - protein, 4 aa, synthetic construct
GGGS

SEQ ID NO: 52 - protein, 5 aa, synthetic construct
GGSGG

SEQ ID NO: 53 - protein, 5 aa, synthetic construct
GSGSG

SEQ ID NO: 54 - protein, 5 aa, synthetic construct
GSGGG

SEQ ID NO: 55 - protein, 5 aa, synthetic construct
GGGGS

SEQ ID NO: 56-60 - skipped (5 entries)

SEQ ID NO: 61 - protein, 16 aa, synthetic construct
RQIKIWFQNRRMKWKK

SEQ ID NO: 62 - RNA, 27 nt, synthetic construct
  source 1..27: mol_type = other RNA
tcctccttgaagtcgatgcccttcagc

SEQ ID NO: 63 - RNA, 27 nt, synthetic construct
  source 1..27: mol_type = other RNA
acgaactccagcaggaccatgtgatcg

SEQ ID NO: 64 - RNA, 27 nt, synthetic construct
  source 1..27: mol_type = other RNA
gaagaagtcgtgctgcttcatgtggtc

SEQ ID NO: 65 - RNA, 30 nt, synthetic construct
  source 1..30: mol_type = other RNA
gcgggtcttgtagttgccgtcgtccttgaa

SEQ ID NO: 66 - RNA, 30 nt, synthetic construct
  source 1..30: mol_type = other RNA
gcggctgaagcactgcacgccgtaggtcag

SEQ ID NO: 67 - RNA, 30 nt, synthetic construct
  source 1..30: mol_type = other RNA
gtgcagatgaacttcagggtcagcttgccg

(19) United States
(12) Patent Application Publication
(10) Pub. No.: US 2025/0313818 A1
(43) Pub. Date: Oct. 9, 2025

(54) CRISPR-Cas Effector Polypeptides and Methods of Use Thereof

(71) Applicant: The Regents of the University of California, Oakland, CA (US)

(72) Inventors: Basem Al-Shayeb, Berkeley, CA (US); Jennifer A. Doudna, Berkeley, CA (US); Jillian F. Banfield, Berkeley, CA (US)

(21) Appl. No.: 18/855,257

(22) PCT Filed: Apr. 10, 2023
(86) PCT No.: PCT/US2023/065592

§ 371 Date: Oct. 8, 2024

Related U.S. Application Data
(60) Provisional application No. 63/329,693, filed on Apr. 11, 2022.

(51) Int. Cl. C12N 9/22; C12N 15/11; C12N 15/88

(52) U.S. Cl. CPC C12N 9/226; C12N 15/111; C12N 15/88; C07K 2319/09; C07K 2319/095; C12N 2310/20

(57) ABSTRACT

The present disclosure provides Type VI CRISPR-Cas effector polypeptides that can, when complexed with a guide nucleic acid, modify a target RNA. A Type VI CRISPR-Cas effector polypeptide of the present disclosure can also provide for detection of nucleic acid by cleavage of non-target RNAs. The present disclosure provides methods of modifying a target RNA, and methods of detecting a nucleic acid.

### CROSS-REFERENCE

This application claims the benefit of U.S. Provisional Patent Application No. 63/329,693, filed Apr. 11, 2022, which application is incorporated herein by reference in its entirety.

### STATEMENT REGARDING FEDERALLY SPONSORED RESEARCH

This invention was made with government support under Grant No. DGE 1752814 awarded by the National Science Foundation. The government has certain rights in the invention.

### INCORPORATION-BY-REFERENCE OF MATERIAL ELECTRONICALLY SUBMITTED

A Sequence Listing is provided herewith as a Sequence Listing XML, “BERK-461WO_SEQ_LIST” created on Apr. 3, 2023 and having a size of 64.9 KB. The contents of the Sequence Listing XML are incorporated by reference herein in their entirety.

### INTRODUCTION

Bacterial adaptive immune systems employ CRISPRs (clustered regularly interspaced short palindromic repeats) and CRISPR-associated (Cas) proteins for RNA-guided nucleic acid cleavage. CRISPR-Cas effector proteins have found use in a variety of applications, including gene editing, nucleic acid detection, and transcription control.

### SUMMARY

The present disclosure provides Type VI CRISPR-Cas effector polypeptides that can, when complexed with a guide nucleic acid, modify a target RNA. A Type VI CRISPR-Cas effector polypeptide of the present disclosure can also provide for detection of nucleic acid by cleavage of non-target RNAs. The present disclosure provides methods of modifying a target RNA, and methods of detecting a nucleic acid.

### BRIEF DESCRIPTION OF THE DRAWINGS

FIG. 1-5 provide amino acid sequences of exemplary Cas13Z polypeptides and repeat sequences of corresponding Cas13Z guide RNAs. Canonical HEPN motifs are underlined (SEQ ID NOs: 1-10, respectively).

FIG. 6 provides an alignment of the amino acid sequences of the Cas13Z polypeptides depicted in FIG. 1-5.

FIG. 7A-7B depicts the effect of a Cas13Z polypeptide/Cas 13Z guide RNA complex on of green fluorescent protein (GFP) transcripts.

FIG. 8A-8C provides amino acid sequences of ADAR polypeptides (SEQ ID NOs: 11-13, respectively).

FIG. 9 provides nucleotide sequences of exemplary green fluorescent protein targeting spacer sequences (SEQ ID NOs: 62-67, respectively).

FIG. 10-13 depict the effect of expressing Cas13Z polypeptides/Cas 13Z guide RNA complexes on green fluorescent protein (GFP) and blue fluorescent protein (BFP) transcripts in mammalian cells.

### DEFINITIONS

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

The term “oligonucleotide” refers to a polynucleotide of between 3 and 100 nucleotides of single- or double-stranded nucleic acid (e.g., DNA, RNA, or a modified nucleic acid). However, for the purposes of this disclosure, there is no upper limit to the length of an oligonucleotide. Oligonucleotides are also known as “oligomers” or “oligos” and can be isolated from genes, transcribed (in vitro and/or in vivo), or chemically synthesized. The terms “polynucleotide” and “nucleic acid” should be understood to include, as applicable to the embodiments being described, single-stranded (such as sense or antisense) and double-stranded polynucleotides.

By “hybridizable” or “complementary” or “substantially complementary” it is meant that a nucleic acid (e.g. RNA, DNA) comprises a sequence of nucleotides that enables it to non-covalently bind, i.e. form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine)(A) pairing with thymidine/thymidine (T), A pairing with uracil/uridine (U), and guanine/guanosine)(G) pairing with cytosine/cytidine (C). In addition, for hybridization between two RNA molecules (e.g., dsRNA), and for hybridization of a DNA molecule with an RNA molecule (e.g., when a DNA target nucleic acid base pairs with a Cas13Z guide RNA, etc.): G can also base pair with U. For example, G/U base-pairing is partially responsible for the degeneracy (i.e., redundancy) of the genetic code in the context of tRNA anti-codon base-pairing with codons in mRNA. Thus, in the context of this disclosure, a G (e.g., of a protein-binding segment (dsRNA duplex) of a Cas13Z guide RNA molecule; of a target nucleic acid base pairing with a Cas13Z guide RNA) is considered complementary to both a U and to C. For example, when a G/U base-pair can be made at a given nucleotide position of a protein-binding segment (e.g., dsRNA duplex) of a Cas13Z guide RNA molecule, the position is not considered to be non-complementary, but is instead considered to be complementary.

Hybridization and washing conditions are well known and exemplified in Sambrook, J., Fritsch, E. F. and Maniatis, T. Molecular Cloning: A Laboratory Manual, Second Edition, Cold Spring Harbor Laboratory Press, Cold Spring Harbor (1989), particularly Chapter 11 and Table 11.1 therein; and Sambrook, J. and Russell, W., Molecular Cloning: A Laboratory Manual, Third Edition, Cold Spring Harbor Laboratory Press, Cold Spring Harbor (2001). The conditions of temperature and ionic strength determine the “stringency” of the hybridization.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. For hybridizations between nucleic acids with short stretches of complementarity (e.g. complementarity over 35 or fewer, 30 or fewer, 25 or fewer, 22 or fewer, 20 or fewer, or 18 or fewer nucleotides) the position of mismatches can become important (see Sambrook et al., supra, 11.7-11.8). Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more). The temperature and wash solution salt concentration may be adjusted as necessary according to factors such as length of the region of complementation and the degree of complementation.

It is understood that the sequence of a polynucleotide need not be 100% complementary to that of its target nucleic acid to be specifically hybridizable or hybridizable. Moreover, a polynucleotide may hybridize over one or more segments such that intervening or adjacent segments are not involved in the hybridization event (e.g., a loop structure or hairpin structure). A polynucleotide can comprise 60% or more, 65% or more, 70% or more, 75% or more, 80% or more, 85% or more, 90% or more, 95% or more, 98% or more, 99% or more, 99.5% or more, or 100% sequence complementarity to a target region within the target nucleic acid sequence to which it will hybridize. For example, an antisense nucleic acid in which 18 of 20 nucleotides of the antisense compound are complementary to a target region, and would therefore specifically hybridize, would represent 90 percent complementarity. In this example, the remaining noncomplementary nucleotides may be clustered or interspersed with complementary nucleotides and need not be contiguous to each other or to complementary nucleotides. Percent complementarity between particular stretches of nucleic acid sequences within nucleic acids can be determined using any convenient method. Exemplary methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones.

“Binding” as used herein (e.g. with reference to an RNA-binding domain of a polypeptide, binding to a target nucleic acid, and the like) refers to a non-covalent interaction between macromolecules (e.g., between a protein and a nucleic acid; between a Cas13Z guide RNA complex and a target nucleic acid; and the like). While in a state of non-covalent interaction, the macromolecules are said to be “associated” or “interacting” or “binding” (e.g., when a molecule X is said to interact with a molecule Y, it is meant the molecule X binds to molecule Y in a non-covalent manner). Not all components of a binding interaction need be sequence-specific (e.g., contacts with phosphate residues in a DNA backbone), but some portions of a binding interaction may be sequence-specific. Binding interactions are generally characterized by a dissociation constant (K_{d}) of less than 10⁻⁶ M, less than 10⁻⁷ M, less than 10⁻⁸ M, less than 10⁻⁹ M, less than 10⁻¹⁰ M, less than 10⁻¹¹ M, less than 10⁻¹² M, less than 10⁻¹³ M, less than 10⁻¹⁴ M, or less than 10⁻¹⁵ M. “Affinity” refers to the strength of binding, increased binding affinity being correlated with a lower K_{d}.

By “binding domain” it is meant a protein domain that is able to bind non-covalently to another molecule. A binding domain can bind to, for example, an RNA molecule (an RNA-binding domain) and/or a protein molecule (a protein-binding domain). In the case of a protein having a protein-binding domain, it can in some cases bind to itself (to form homodimers, homotrimers, etc.) and/or it can bind to one or more regions of a different protein or proteins.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various methods and computer programs (e.g., BLAST, T-COFFEE, MUSCLE, MAFFT, Phyre2, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/, http://www.sbg.bio.ic.ac.uk/˜phyre2/. See, e.g., Altschul et al. (1990), J. Mol. Biol. 215:403-10.

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, microRNA (miRNA), a “non-coding” RNA (ncRNA), a Cas13Z guide RNA, etc.).

The terms “DNA regulatory sequences,” “control elements,” and “regulatory elements,” used interchangeably herein, refer to transcriptional and translational control sequences, such as promoters, enhancers, polyadenylation signals, terminators, protein degradation signals, and the like, that provide for and/or regulate transcription of a non-coding sequence (e.g., Cas13Z guide RNA) or a coding sequence (e.g., Cas13Z polypeptide) and/or regulate translation of an encoded polypeptide.

As used herein, a “promoter sequence” is a DNA regulatory region capable of binding RNA polymerase and initiating transcription of a downstream (3′ direction) coding or non-coding sequence. For purposes of the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present disclosure.

The term “naturally-occurring” or “unmodified” or “wild type” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature and which has not been intentionally modified by a human in the laboratory is wild type (and naturally occurring).

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). Alternatively, DNA sequences encoding RNA (e.g., Cas13Z guide RNA) that is not translated may also be considered recombinant. Thus, e.g., the term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques. When a recombinant polynucleotide encodes a polypeptide, the sequence of the encoded polypeptide can be naturally occurring (“wild type”) or can be a variant (e.g., a mutant) of the naturally occurring sequence. Thus, the term “recombinant” polypeptide does not necessarily refer to a polypeptide whose sequence does not naturally occur. Instead, a “recombinant” polypeptide is encoded by a recombinant DNA sequence, but the sequence of the polypeptide can be naturally occurring (“wild type”) or non-naturally occurring (e.g., a variant, a mutant, etc.). Thus, a “recombinant” polypeptide is the result of human intervention, but may be a naturally occurring amino acid sequence.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter. “Operably linked” refers to a juxtaposition wherein the components so described are in a relationship permitting them to function in their intended manner. For instance, a promoter is operably linked to a coding sequence if the promoter affects its transcription or expression.

The terms “recombinant expression vector,” or “DNA construct” are used interchangeably herein to refer to a DNA molecule comprising a vector and one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

Any given component, or combination of components can be unlabeled, or can be detectably labeled with a label moiety. In some cases, when two or more components are labeled, they can be labeled with label moieties that are distinguishable from one another.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

“Heterologous,” as used herein, refers to a nucleotide or polypeptide sequence that is not found in the native nucleic acid or protein, respectively. For example, relative to a Cas13Z polypeptide of the present disclosure, a heterologous polypeptide comprises an amino acid sequence from a protein other than the Cas13Z polypeptide. As another example, a Cas 13Z polypeptide of the present disclosure can be fused to an active domain from a non-CRISPR/Cas effector protein (e.g., a demethylase), and the sequence of the active domain could be considered a heterologous polypeptide (it is heterologous to the Cas13Z polypeptide). As another example, a guide sequence of a guide RNA that is heterologous to a protein-binding sequence of a guide RNA is a guide sequence that is not found in nature together with the protein-binding sequence.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a Cas 13Z polypeptide” includes a plurality of such Cas13Z polypeptides and reference to “the guide RNA” includes reference to one or more guide RNAs and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides CRISPR-Cas effector polypeptides, nucleic acids encoding the CRISPR-Cas effector polypeptides, and systems and kits comprising the CRISPR-Cas effector polypeptides. The present disclosure provides methods of editing a target RNA. The present disclosure provides methods of detecting an RNA.

A CRISPR-Cas effector polypeptide of the present disclosure finds use in a number of applications, including RNA detection, detection of DNA (via detection of an RNA transcript of the DNA), detection of transcriptional activity, RNA knockdown, RNA editing, RNA tracking, transcriptome editing, epitranscriptome editing, translational upregulation, epi-transcriptomic reading and writing via N6-Methyladenosinc, and isoform modulation.

### Crispr-Cas Effector Polypeptides

The present disclosure provides CRISPR-Cas effector polypeptides, which are referred to herein as “Cas13Z” polypeptides. A CRISPR-Cas effector polypeptide of the present disclosure is a Type VI CRISPR-Cas effector polypeptide. A Cas13Z polypeptide of the present disclosure binds to a guide nucleic acid (e.g., a guide RNA), which guide nucleic acid is referred to herein as a “Cas13Z guide RNA.” A Cas13Z polypeptide binds to a Cas13Z guide RNA, is guided to a target RNA, and is thereby activated. A Cas13Z polypeptide can include two HEPN domains: HEPN1 and HEPN2. If the HEPN1 and HEPN2 domains of the Cas13Z polypeptide are intact, once activated, the Cas13Z polypeptide cleaves the target RNA; such cleavage is referred to as “cis cleavage.” Upon activation, a Cas13Z polypeptide can also cleave non-target RNAs in a sequence-non-specific manner; such cleavage is referred to as “trans cleavage.”

A Cas 13Z polypeptide of the present disclosure can have a length of from 790 amino acids to 910 amino acids; e.g., a Cas13Z polypeptide can have a length of from 790 amino acids to 795 amino acids, from 795 amino acids to 800 amino acids, from 800 amino acids to 825 amino acids, from 825 amino acids to 850 amino acids, from 850 amino acids to 875 amino acids, from 875 amino acids to 880 amino acids, from 880 amino acids to 885 amino acids, from 885 amino acids to 890 amino acids, from 890 amino acids to 895 amino acids, from 895 amino acids to 900 amino acids, from 900 amino acids to 905 amino acids, or from 905 amino acids to 910 amino acids. In some cases, a Cas13Z polypeptide has a length of 798 amino acids. In some cases, a Cas13Z polypeptide has a length of 871 amino acids. In some cases, a Cas13Z polypeptide has a length of 878 amino acids. In some cases, a Cas 13Z polypeptide has a length of 892 amino acids. In some cases, a Cas13Z polypeptide has a length of 901 amino acids.

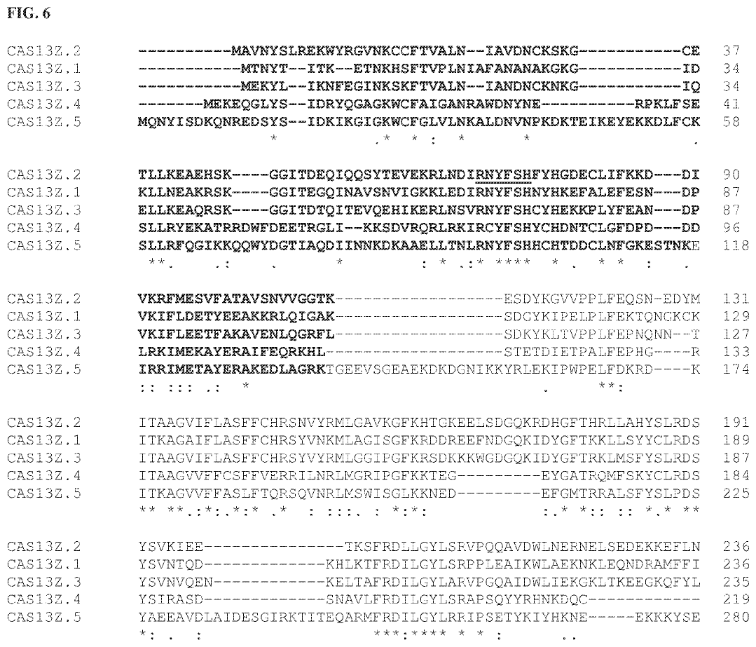

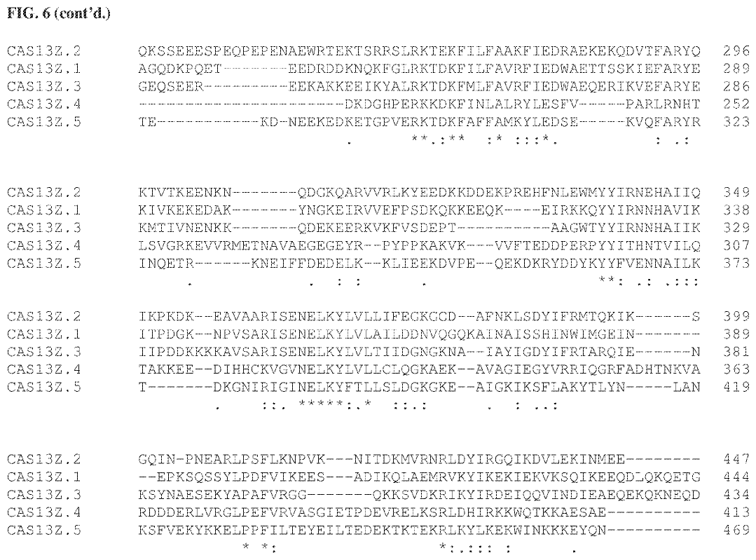

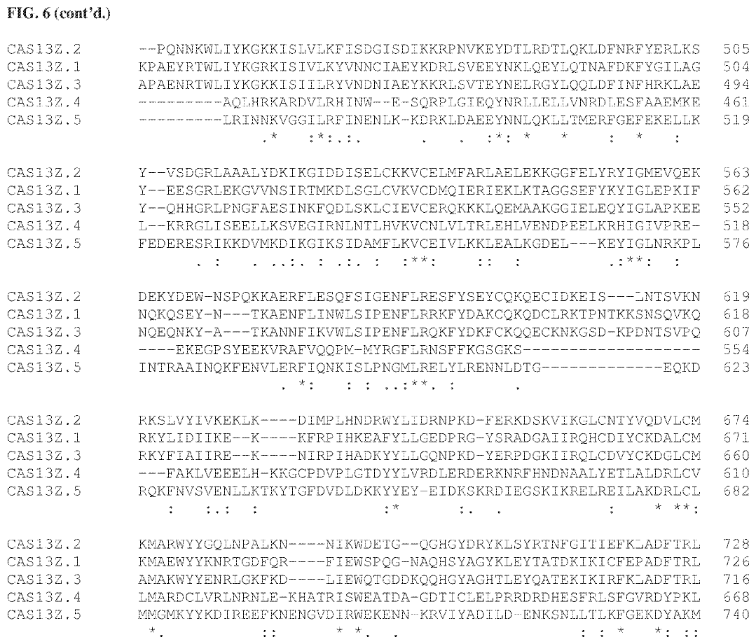

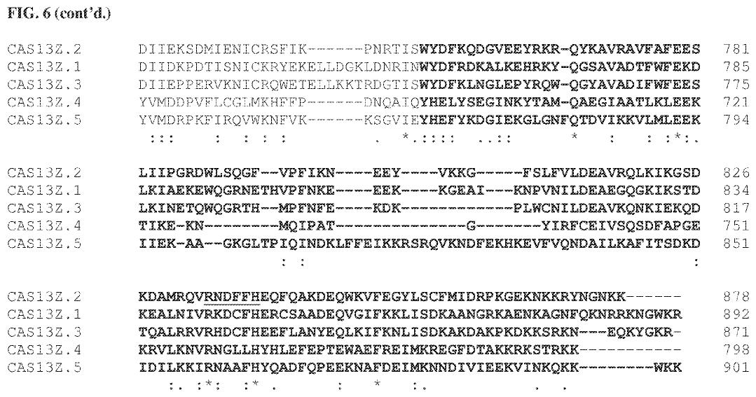

As noted above, a Cas13Z polypeptide comprises a HEPN1 domain and a HEPN2 domain, where each HEPN domain includes a canonical HEPN motif. As illustrated in FIG. 6, the HEPN1 domain is shown in bold. For example, the HEPN1 domain of Cas13Z.2 is: MAVNYSLREKWYRGVNKCCFTVALNIAVDNCKSKGCETLLKEAEHSKGGITDEQIQQSYTEVE KRLNDIRNYFSHFYHGDECLIFKKDDIVKRFMESVFATAVSNVVGGTK (SEQ ID NO:14), with the canonical HEPN motif underlined; and the HEPN2 domain of Cas13Z.2 is: WYDFKQDGVEEYRKRQYKAVRAVFAFEESLIIPGRDWLSQGFVPFIKNEEYVKKGFSLFVLDEA VRQLKIKGSDKDAMRQVRNDFFHEQFQAKDEQWKVFEGYLSCFMIDRPKGEKNKKRYNGNK K (SEQ ID NO:15), with the canonical HEPN motif underlined.

Each HEPN domain includes a canonical HEPN motif, where the canonical HEPN motif is R(X)nH, where n is an integer from 3 to 5, and where X is any amino acid. In some cases, a HEPN domain present in a Cas13Z polypeptide includes a HEPN motif RX₁X₂X₃X₄H, where X₁ is N, H, C, or K, and where X₂, X₃, and X₄ are each independently any amino acid. In some cases, the HEPN1 domain comprises the amino acid sequence RNYFSH (SEQ ID NO:16) or RCYFSH (SEQ ID NO:17). In some cases, the HEPN2 domain comprises the amino acid sequence RX₁X₂X₃X₄H, where X₁ is N, K, or H; X₂ is D, G, or A; X₃ is F, C, L, or A; and X₄ is F or L. In some cases, the HEPN2 domain comprises the amino acid sequence RNDFFH (SEQ ID NO:18). In some cases, the HEPN2 domain comprises the amino acid sequence RKDCFH (SEQ ID NO:19). In some cases, the HEPN2 domain comprises the amino acid sequence RHDCFH (SEQ ID NO:20). In some cases, the HEPN2 domain comprises the amino acid sequence RNGLLH (SEQ ID NO:21). In some cases, the HEPN2 domain comprises the amino acid sequence RNAAFH (SEQ ID NO:22).

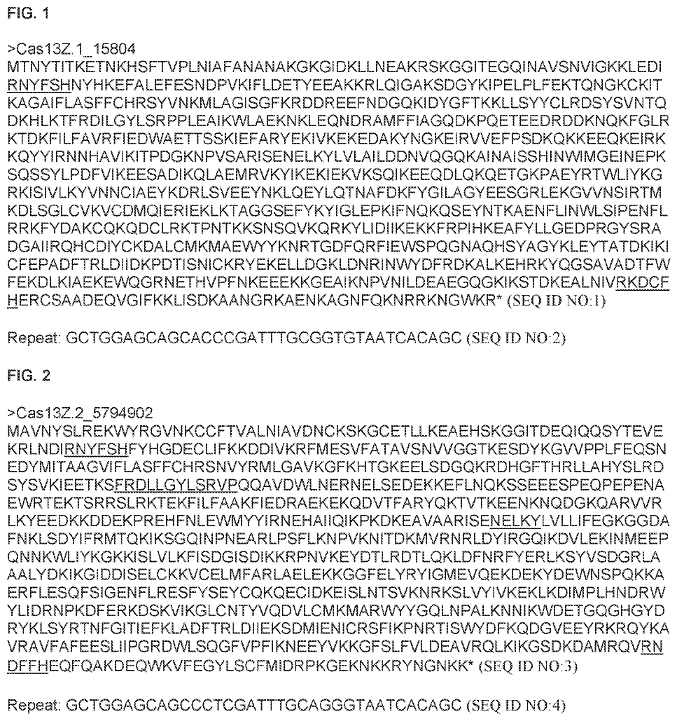

FIG. 6 provides an amino acid sequence alignment of 5 Cas13Z polypeptides. The alignment indicates the positions of the canonical HEPN1 motif and the canonical HEPN2 motif. The alignment also indicates other conserved amino acid sequences; these include, e.g., i) the sequence FRD (I/L) LGYL (S/R) R (V/P/A/I) P (e.g., at amino acids 202-213 of the amino acid sequence of Cas13Z.2 shown in FIG. 2, or corresponding positions in another Cas13Z polypeptide); and ii) the sequence NELKY (e.g., at amino acids 365-369 of the amino acid sequence of Cas13Z.2 shown in FIG. 2, or corresponding positions in another Cas13Z polypeptide). The corresponding amino acid positions are apparent from the alignment provided in FIG. 6. Other conserved amino acids are apparent from the alignment provided in FIG. 6.

In some cases, a Cas13Z polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to a contiguous stretch of from 850 amino acids to 892 amino acids of the amino acid sequence depicted in FIG. 1 (designated “Cas13Z.1_15804” in FIG. 1 and also referred to herein as “Cas13Z.1”). In some cases, the Cas13Z polypeptide has a length of from 850 amino acids to 892 amino acids (e.g., from 850 to 875, from 875 to 880, from 880 to 885, from 885 to 890, or from 890 to 892 amino acids). In some cases, the Cas13Z polypeptide has a length of 892 amino acids.

In some cases, a Cas 13Z polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to a contiguous stretch of from 840 amino acids to 878 amino acids of the amino acid sequence depicted in FIG. 2 (designated “Cas13Z.2_5794902” in FIG. 2 and also referred to herein as “Cas13Z.2”). In some cases, the Cas13Z polypeptide has a length of from 840 amino acids to 878 amino acids (e.g., from 840 to 845, from 845 to 850, from 850 to 855, from 855 to 860, from 860 to 865, from 865 to 870, or from 870 to 878 amino acids). In some cases, the Cas13Z polypeptide has a length of 878 amino acids.

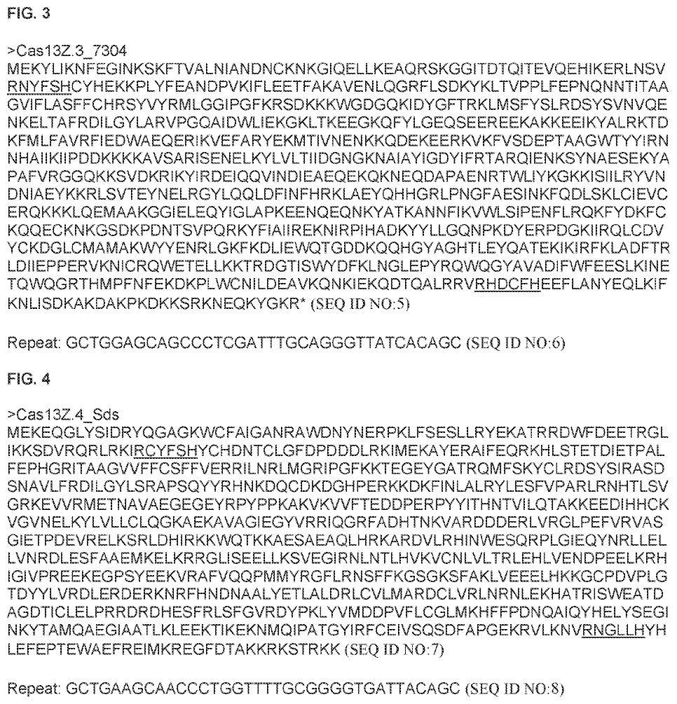

In some cases, a Cas13Z polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to a contiguous stretch of from 840 amino acids to 871 amino acids of the amino acid sequence depicted in FIG. 3 (designated “Cas13Z.3_7304” in FIG. 3 and also referred to herein as “Cas13Z.3”). In some cases, the Cas13Z polypeptide has a length of from 840 amino acids to 871 amino acids (e.g., from 840 to 845, from 845 to 850, from 850 to 855, from 855 to 860, from 860 to 865, or from 865 to 871 amino acids). In some cases, the Cas13Z polypeptide has a length of 871 amino acids.

In some cases, a Cas13Z polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to a contiguous stretch of from 750 amino acids to 798 amino acids of the amino acid sequence depicted in FIG. 4 (designated “Cas13Z.4_Sds” in FIG. 4 and also referred to herein as “Cas13Z.4”). In some cases, the Cas13Z polypeptide has a length of from 750 to 755, from 755 to 780, from 780 to 785, from 785 to 790, or from 790 to 798 amino acids). In some cases, the Cas13Z polypeptide has a length of 798 amino acids.

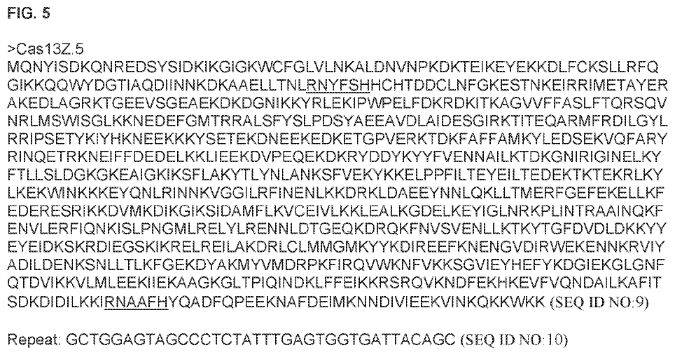

In some cases, a Cas13Z polypeptide of the present disclosure comprises an amino acid sequence having at least 50%, at least 55%, at least 60%, at least 65%, at least 70%, at least 75%, at least 80%, at least 85%, at least 90%, at least 95%, at least 98%, at least 99%, or 100%, amino acid sequence identity to a contiguous stretch of from 850 amino acids to 901 amino acids of the amino acid sequence depicted in FIG. 5 (designated “Cas13Z.5” in FIG. 5 and also referred to herein as “Cas13Z.5”). In some cases, the Cas13Z polypeptide has a length of from 850 to 860, from 860 to 870, from 870 to 880, from 880 to 890, or from 890 to 901 amino acids). In some cases, the Cas13Z polypeptide has a length of 901 amino acids.

Variant Cas13Z polypeptides

The term “Cas13Z polypeptide” encompasses variants, e.g., variants having reduced catalytic activity compared to the catalytic activity of a Cas13Z polypeptide comprising an amino acid sequence depicted in any one of FIG. 1-5. In some cases, a variant Cas13Z polypeptide retains the ability, when complexed with a Cas13Z guide RNA, to bind to a target RNA. In some cases, a variant Cas13Z polypeptide, when complexed with a Cas13Z guide RNA: i) retains the ability to bind to a target RNA; and ii) exhibits reduced catalytic activity (e.g., cleavage of a target and/or a non-target RNA) compared to the catalytic activity of a Cas13Z polypeptide comprising an amino acid sequence depicted in any one of FIG. 1-5).

In some cases, a variant Cas 13Z polypeptide exhibits reduced (or undetectable) nuclease activity. For example, in some cases, a variant Cas13Z protein lacks a catalytically active HEPN1 domain. As another example, a variant Cas13Z protein lacks a catalytically active HEPN2 domain. In some cases, a variant Cas13Z protein lacks a catalytically active HEPN1 domain and lacks a catalytically active HEPN2 domain. In some cases, a variant Cas13Z polypeptide comprises substitutions of 1, 2, 3, or 4 of amino acids R67, H72, R842, and H847, based on the amino acid number of the Cas13Z.1 polypeptide depicted in FIG. 1, or the corresponding amino acids of another Cas13Z polypeptide. Corresponding amino acids can be readily determined by amino acid sequence alignment; see, e.g., FIG. 6. In some cases, the HEPN1 domain and/or the HEPN2 domain comprises a deletion of one or more amino acids. In some cases, the canonical motif of the HEPN1 domain and/or the HEPN2 domain is deleted. For example, in some cases, a variant Cas13Z polypeptide does not comprise the amino acid sequence RNYFSH (SEQ ID NO:16) or RCYFSH (SEQ ID NO:17). As another example, in some cases, a variant Cas13Z polypeptide does not comprise the sequence RNDFFH (SEQ ID NO:18), RKDCFH (SEQ ID NO: 19), RHDCFH (SEQ ID NO:20), RNGLLH (SEQ ID NO:21), or RNAAFH (SEQ ID NO:22).

Fusion polypeptides

In some cases, a Cas 13Z polypeptide of the present disclosure is part of a fusion polypeptide comprising: i) a Cas13Z polypeptide; and ii) one or more heterologous polypeptides, where a hctcrologous polypeptide is also referred to as a “fusion partner.” In some cases, the Cas 13Z polypeptide of the Cas13Z fusion polypeptide is a catalytically active Cas13Z polypeptide. In some cases, the Cas13Z polypeptide of the Cas13Z fusion polypeptide exhibits reduced catalytic activity compared to the catalytic activity of a Cas13Z polypeptide having an amino acid sequence depicted in any one of FIG. 1-5. In some cases, the Cas13Z polypeptide of the Cas13Z fusion polypeptide is a catalytically inactive Cas13Z polypeptide. In some cases, a Cas13Z polypeptide present in a Cas13Z polypeptide i) retains the ability to bind to a target RNA; and ii) exhibits reduced catalytic activity (e.g., cleavage of a target and/or a non-target RNA) compared to the catalytic activity of a Cas13Z polypeptide comprising an amino acid sequence depicted in any one of FIG. 1-5).

Suitable heterologous polypeptides (fusion partners) include effector polypeptides. Exemplary effector polypeptides include, e.g., polypeptides that can cleave RNA (e.g., a PIN endonuclease, an NYN domain, an SMR domain from SOT1, or an RNase domain from a Staphylococcal nuclease); polypeptides that can affect RNA stability (e.g., tristetraprolin (TTP) or domains from UPF1, EXOSC5, and STAU1); polypeptides that can modify a nucleotide or ribonucleotide (e.g., a cytidine deaminase, PPR protein, adenosine deaminase, an adenosine deaminase acting on RNA (ADAR) family protein, or an APOBEC family protein); polypeptides that can activate translation (e.g., eIF4E and other translation initiation factors, a domain of the yeast poly (A)-binding protein or GLD2), those that can repress translation (e.g., Pumilio or FBF PUF proteins, deadenylases, CAF1, Argonaute proteins); polypeptides that can methylate RNA (e.g., domains from m6A methyltransferase factors such as METTL14, METTL3, or WTAP); polypeptides that can demethylate RNA (e.g., human alkylation repair homolog 5 or Alkbh5); polypeptides that can affect splicing (e.g., the RS-rich domain of SRSF1, the Gly-rich domain of hnRNP A1, the alanine-rich motif of RBM4, or the proline-rich motif of DAZAP1); polypeptides that can enable affinity purification or immunoprecipitation (e.g., FLAG, hemagglutinin (HA), biotin, or HALO tags); and polypeptides that can enable proximity-based protein labeling and identification (e.g., a biotin ligase (such as BirA) or a peroxidase (such as APEX2) in order to biotinylate proteins that interact with the target RNA).

Suitable heterologous polypeptides include splicing factors (e.g., RS domains); protein translation components (e.g., translation initiation, elongation, and/or release factors; e.g., eIF4G); RNA methylases; RNA editing enzymes (e.g., RNA deaminases, e.g., ADAR polypeptides, including A to I and/or C to U editing enzymes); helicases; RNA-binding proteins; and the like.

Suitable heterologous polypeptides include, e.g., endonucleases (for example RNase III, the CRR22 DYW domain, Dicer, and PIN(PilT N-terminus) domains from proteins such as SMG5 and SMG6); proteins and protein domains responsible for stimulating RNA cleavage (for example CPSF, CstF, CFIm and CFIIm); exonucleases (for example XRN-1 or Exonuclease T); deadenylases (for example HNT3); proteins and protein domains responsible for nonsense mediated RNA decay (for cxamplc UPF1, UPF2, UPF3, UPF3b, RNP S1, Y14, DEK, REF2, and SRm160); protcins and protein domains responsible for stabilizing RNA (for example PABP); proteins and protein domains responsible for repressing translation (for example Ago2 and Ago4); proteins and protein domains responsible for stimulating translation (for example Staufen); proteins and protein domains responsible for (e.g., capable of) modulating translation (e.g., translation factors such as initiation factors, elongation factors, release factors, etc., e.g., eIF4G); proteins and protein domains responsible for polyadenylation of RNA (for example PAP1, GLD-2, and Star-PAP); proteins and protein domains responsible for polyuridinylation of RNA (for example CI D1 and terminal uridylate transferase); proteins and protein domains responsible for RNA localization (for example from IMP1, ZBP1, She2p, She3p, and Bicaudal-D); proteins and protein domains responsible for nuclear retention of RNA (for example Rrp6); proteins and protein domains responsible for nuclear export of RNA (for example TAP, NXF1, THO, TREX, REF, and Aly); proteins and protein domains responsible for repression of RNA splicing (for example PTB, Sam68, and hnRNP A1); proteins and protein domains responsible for stimulation of RNA splicing (for example Serine/Arginine-rich (SR) domains); proteins and protein domains responsible for reducing the efficiency of transcription (for example FUS (TLS)); and proteins and protein domains responsible for stimulating transcription (for example CDK7 and HIV Tat). Alternatively, the effector domain may be selected from the group comprising endonucleases; proteins and protein domains capable of stimulating RNA cleavage; exonucleases; deadenylases; proteins and protein domains having nonsense mediated RNA decay activity; proteins and protein domains capable of stabilizing RNA; proteins and protein domains capable of repressing translation; proteins and protein domains capable of stimulating translation; proteins and protein domains capable of modulating translation (e.g., translation factors such as initiation factors, elongation factors, release factors, etc., e.g., eIF4G); proteins and protein domains capable of polyadenylation of RNA; proteins and protein domains capable of polyuridinylation of RNA; proteins and protein domains having RNA localization activity; proteins and protein domains capable of nuclear retention of RNA; proteins and protein domains having RNA nuclear export activity; proteins and protein domains capable of repression of RNA splicing; proteins and protein domains capable of stimulation of RNA splicing; proteins and protein domains capable of reducing the efficiency of transcription; and proteins and protein domains capable of stimulating transcription. Another suitable fusion partner is a PUF RNA-binding domain, which is described in more detail in WO2012068627.

Some RNA splicing factors that can be used (in whole or as fragments thereof) as fusion partners for a Cas13Z polypeptide have modular organization, with separate sequence-specific RNA binding modules and splicing effector domains. For example, members of the Serine/Arginine-rich (SR) protein family contain N-terminal RNA recognition motifs (RRMs) that bind to exonic splicing enhancers (ESEs) in pre-mRNAs and C-terminal RS domains that promote exon inclusion. As another example, the hnRNP protein hnRNP Al binds to exonic splicing silencers (ESSs) through its RRM domains and inhibits exon inclusion through a C-terminal Glycine-rich domain. Some splicing factors can regulate alternative use of splice site (ss) by binding to regulatory sequences between the two alternative sites. For example, ASF/SF2 can recognize ESEs and promote the use of intron proximal sites, whereas hnRNP A1 can bind to ESSs and shift splicing towards the use of intron distal sites. One application for such factors is to generate ESFs that modulate alternative splicing of endogenous genes, particularly disease associated genes. For example, Bcl-x pre-mRNA produces two splicing isoforms with two alternative 5′ splice sites to encode proteins of opposite functions. The long splicing isoform Bcl-xL is a potent apoptosis inhibitor expressed in long-lived postmitotic cells and is up-regulated in many cancer cells, protecting cells against apoptotic signals. The short isoform Bcl-xS is a pro-apoptotic isoform and expressed at high levels in cells with a high turnover rate (e.g., developing lymphocytes). The ratio of the two Bcl-x splicing isoforms is regulated by multiple cis-elements that are located in either the core exon region or the exon extension region (i.e., between the two alternative 5′ splice sites). For more examples, see WO2010075303.

In some cases, the heterologous fusion polypeptide is an RNA methyltransferase, an RNA demethylase, an RNA splicing modifier, a localization factor, or a translation modification factor. In some cases, the heterologous fusion polypeptide is a methyltransferase (e.g., METTL3, METTL14, or METTL3/METTL14). In some cases, the methyltransferase is capable of making an N°-methyladenosine modification in an RNA. In some cases, the methyltransferase is capable of making a 1-methyladenosine modification in an RNA. In some cases, the methyltransferase is capable of making a 5-hydroxymethylcytidine modification in an RNA. In some cases, the heterologous fusion polypeptide is a demethylase (e.g., ALKBH5 or FTO).

In some cases, a Cas13Z fusion polypeptide comprises: a) a Cas13Z polypeptide; and b) an ADAR polypeptide (as the fusion partner)(e.g., an ADAR1 polypeptide, an ADAR2 polypeptide; or an ADAR3 polypeptide). The term “adenosine deaminases acting on RNA” or “ADAR” as used herein can refer to an adenosine deaminase that can convert adenosines (A) to inosines (I) in an RNA moleculc.

An ADAR polypeptide can comprise a catalytic domain. An ADAR1 catalytic domain can comprise a catalytic domain comprising an amino acid sequence having at least 50%, at least 60%, at least 70%, at least 75%, at least 80%, at least 85%, at least 90%, at least 95%, at least 98%, at least 99%, or 100%, amino acid sequence identity the following amino acid sequence:

(SEQ ID NO: 23)
KAERMGFTEVTPVTGASLRRTMLLLSRSPEAQPKTLPLTGSTFHDQIAML
SHRCFNTLTNSFQPSLLGRKILAAIIMKKDSEDMGVVVSLGTGNRCVKGD
SLSLKGETVNDCHAEIISRRGFIRFLYSELMKYNSQTAKDSIFEPAKGGE
KLQIKKTVSFHLYISTAPCGDGALFDKSCSDRAMESTESRHYPVFENPKQ
GKLRTKVENGEGTIPVESSDIVPTWDGIRLGERLRTMSCSDKILRWNVLG
LQGALLTHFLQPIYLKSVTLGYLFSQGHLTRAICCRVTRDGSAFEDGLRH
PFIVNHPKVGRVSIYDSKRQSGKTKETSVNWCLADGYDLEILDGTRGTVD
GPRNELSRVSKKNIFLLFKKLCSFRYRRDLLRLSYGEAKKAARDYETAKN
YFKKGLKDMGYGNVVISKPQEEKNFYLCPV.

An ADAR2 catalytic domain can comprise a catalytic domain comprising an amino acid sequence having at least 50%, at least 60%, at least 70%, at least 75%, at least 80%, at least 85%, at least 90%, at least 95%, at least 98%, at least 99%, or 100%, amino acid sequence identity the following amino acid sequence:

(SEQ ID NO: 24)
QLHLPQVLADAVSRLVLGKFGDLTDNFSSPHARRKVLAGVVMTTGTDVKD
AKVISVSTGTKCINGEYMSDRGLALNDCHAEIISRRSLLRFLYTQLELYL
NNKDDQKRSIFQKSERGGFRLKENVQFHLYISTSPCGDARIFSPHEPILE
EPADRHPNRKARGQLRTKIESGEGTIPVRSNASIQTWDGVLQGERLLTMS
CSDKIARWNVVGIQGSLLSIFVEPIYFSSIILGSLYHGDHLSRAMYQRIS
NIEDLPPLYTLNKPLLSGISNAEARQPGKAPNFSVNWTVGDSAIEVINAT
TGKDELGRASRLCKHALYCRWMRVHGKVPSHLLRSKITKPNVYHESKLAA
KEYQAAKARLFTAFIKAGLGAWVEKPTEQDQFSLT.

An ADAR polypeptide can comprise a double-stranded RNA binding domain (dsRBD). The dsRBD of an ADAR polypeptide can comprise an amino acid sequence having at least 50%, at least 60%, at least 70%, at least 75%, at least 80%, at least 85%, at least 90%, at least 95%, at least 98%, at least 99%, or 100%, amino acid sequence identity the following amino acid sequence include the following amino acid sequence:

(SEQ ID NO: 25)
MDIEDEENMSSSSTDVKENRNLDNVSPKDGSTPGPGEGSQLSNGGGGGPG
RKRPLEEGSNGHSKYRLKKRRKTPGPVLPKNALMQLNEIKPGLQYTLLSQ
TGPVHAPLFVMSVEVNGQVFEGSGPTKKKAKLHAAEKALRSFVQFPNASE
AHLAMGRTLSVNTDFTSDQADFPDTLFNGFETPDKAEPPFYVGSNGDDSF
SSSGDLSLSASPVPASLAQPPLPVLPPFPPPSGKNPVMILNELRPGLKYD
ELSESGESHAKSFVMSVVVDGQFFEGSGRNKKLAKARAAQSALAAIFN.

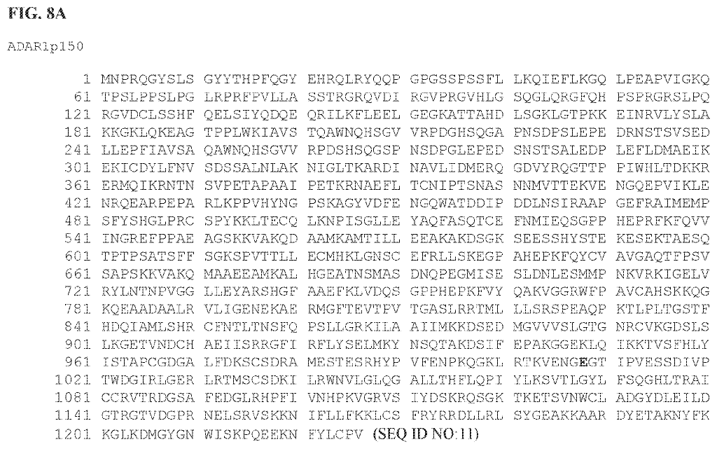

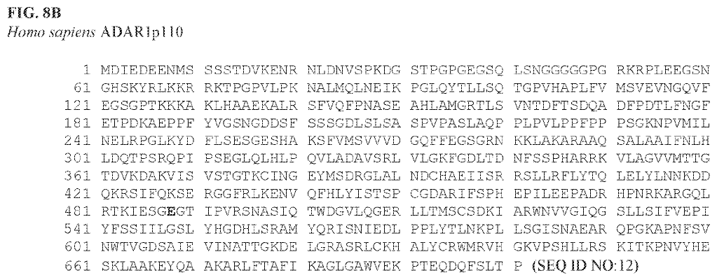

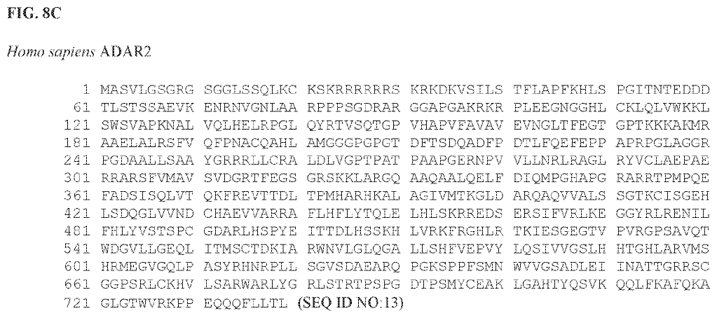

In some cases, an ADAR1 polypeptide comprises an amino acid sequence having at least 50%, at least 60%, at least 70%, at least 75%, at least 80%, at least 85%, at least 90%, at least 95%, at least 98%, at least 99%, or 100%, amino acid sequence identity to the ADAR1 amino acid sequence depicted in FIG. 8A. In some cases, an ADAR1 polypeptide comprises an amino acid sequence having at least 50%, at least 60%, at least 70%, at least 75%, at least 80%, at least 85%, at least 90%, at least 95%, at least 98%, at least 99%, or 100%, amino acid sequence identity to the ADAR1 amino acid sequence depicted in FIG. 8B. In some cases, an ADAR2 polypeptide comprises an amino acid sequence having at least 50%, at least 60%, at least 70%, at least 75%, at least 80%, at least 85%, at least 90%, at least 95%, at least 98%, at least 99%, or 100%, amino acid sequence identity to the ADAR2 amino acid sequence depicted in FIG. 8C.

The term “ADAR” encompasses wild-type ADAR polypeptides (ADAR polypeptides having naturally-occurring amino acid sequences) and variants, e.g., “promiscuous” ADAR variants, and variants having altered enzymatic activity compared to a naturally-occurring ADAR polypeptide. Examples of variant ADAR polypeptides include E488Q and E1008Q variants of ADAR1, E488Q and E1008Q variants of ADAR2, and the like.

In some cases, a “promiscuous” ADAR2 variant comprises the following amino acid sequence:

(SEQ ID NO: 26)
MLRSFVQFPNASEAHLAMGRTLSVNTDFTSDQADFPDTLFNGFETPDKAE
PPFYVGSNGDDSFSSSGDLSLSASPVPASLAQPPLPVLPPFPPPSGKNPV
MILNELRPGLKYDFLSESGESHAKSFVMSVVVDGQFFEGSGRNKKLAKAR
AAQSALAAIFNLHLDQTPSRQPIPSEGLQLHLPQVLADAVSRLVLGKFGD
LTDNFSSPHARRKVLAGVVMTTGTDVKDAKVISVSTGTKCINGEYMSDRG
LALNDCHAEIISRRSLLRFLYTQLELYLNNKDDQKRSIFQKSERGGFRLK
ENVQFHLYISTSPCGDARIFSPHEPILEEPADRHPNRKARGQLRTKIESG
EGTIPVRSNASIQTWDGVLQGERLLTMSCSDKIARWNVVGIQGSLLSIFV
EPIYFSSIILGSLYHGDHLSRAMYQRISNIEDLPPLYTLNKPLLSGISNA
EARQPGKAPNFSVNWTVGDSAIEVINATTGKDELGRASRLCKHALYCRWM
RVHGKVPSHLLRSKITKPNVYHESKLAAKEYQAAKARLFTAFIKAGLGAW
VEKPTEQDQFSLTP.

In some cases, the ADAR polypeptide is a hyperactive Q mutant of an ADAR1 deaminase or an ADAR2 deaminase (e.g., human ADAR1p150, E1008Q; human ADAR1p110 E448Q; human ADAR2 E488Q; human ADAR2 E448Q/T375G).

In some cases, a Cas13Z fusion polypeptide comprises: a) a Cas13Z polypeptide; and b) a cytidine deaminase. In some cases, the cytidine deaminase is an activation-induced cytidine deaminase (AID). In some cases, a Cas13Z fusion polypeptide comprises: a) a Cas13Z polypeptide; and b) an Apolipoprotein B mRNA Editing Catalytic Polypeptide-like (APOBEC) polypeptide (e.g., an APOBEC1, APOBEC2, APOBEC3A, APOBEC3B, APOBEC3C, APOBEC3E, APOBEC3F, APOBEC3G, APOBEC3H, or APOBEC4 polypeptide).

In some cases, a suitable cytidine deaminase comprises an amino acid sequence having at least 80%, at least 85%, at least 90%, at least 95%, at least 98%, at least 99%, or 100%, amino acid sequence identity to the following amino acid sequence:

(SEQ ID NO: 27)
MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKDYFYCWNT
FVENHERTFKAWEGLHENSVRLSRQLRRILLPLYEVDDLRDAFRTLGL.

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SFSLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKENHERTFK AWEGLHENSV RLSRQLRRIL LPLYEVDDLR DAFRTLGL (SEQ ID NO:28).

In some cases, a suitable cytidine deaminase is an AID and comprises an amino acid sequence having at least 80%, at least 85%, at least 90%, at least 95%, at least 98%, at least 99%, or 100%, amino acid sequence identity to the following amino acid sequence: MDSLLMNRRK FLYQFKNVRW AKGRRETYLC YVVKRRDSAT SESLDFGYLR NKNGCHVELL FLRYISDWDL DPGRCYRVTW FTSWSPCYDC ARHVADFLRG NPNLSLRIFT ARLYFCEDRK AEPEGLRRLH RAGVQIAIMT FKDYFYCWNT FVENHERTFK AWEGLHENSV RLSRQLRRIL LPLYEVDDLR DAFRTLGL (SEQ ID NO: 27).

A Cas13Z fusion polypeptide can function as a transcriptional sensor, e.g., to sense an RNA transcript. For example, the transcriptional sensor can include: i) a Cas13Z polypeptide (e.g., a Cas13Z polypeptide with a mutated HEPN domain); ii) at least one gRNA containing at least one spacer sequence specific for a target RNA; and iii) an effector polypeptide such as: 1) an optionally split fluorescent protein or probe (e.g., a split Venus fluorescent protein, a split green fluorescent protein (GFP), a split enhanced GFP, a split mCherry, a split super-folder mCherry, and other fluorescent protein variants such as cyan fluorescent protein (CFP), yellow fluorescent protein (YFP), red fluorescent protein (RFP), and derivatives or fragments thereof); 2) an optionally split luminescent protein or probe (e.g. Gaussia, Firefly, NanoLuc, or Renilla variants); 3) an optionally split enzyme (e.g., ubiquitin or a tobacco etch virus (TEV) protease); 4) a FRET-compatible protein pair; 5) one or more transcription factor(s) fused to a Cas13Z polypeptide via a cleavable linker (e.g., an artificial GAL4, zinc finger, transcriptional activator like effector (TALE), or TetR-based transcription factor or an endogenous transcription factor); 6) a split intein that trans-splices a protein to restore its function such as a transcription factor (e.g., an intein from Rhodothermus marinus or DnaE); 7) a kinase-substrate pair that activates upon phosphorylation (e.g., TYK2-STAT3); 8) one, two, or more monomers that activate upon dimerization or multimerization (e.g., caspase 9); or 9) one or more proteins that induce conformational and functional change upon interaction. As an example, the spatial proximity of a Cas13Z polypeptide and a Cas13Z gRNA following binding a particular RNA transcript would activate the fusion partner, resulting in a detectable signal or detectable activity in the cell; such detectable signal or detectable activity would indicate the presence of the RNA transcript.

In some cases, a heterologous polypeptide (a fusion partner) provides for subcellular localization, i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an endoplasmic reticulum (ER) retention signal, and the like). In some cases, a Cas13Z fusion polypeptide does not include an NLS.

Non-limiting examples of NLSs include an NLS sequence derived from: the NLS of the SV40 virus large T-antigen, having the amino acid sequence PKKKRKV (SEQ ID NO:29); the NLS from nucleoplasmin (e.g., the nucleoplasmin bipartite NLS with the sequence KRPAATKKAGQAKKKK (SEQ ID NO:30)); the c-myc NLS having the amino acid sequence PAAKRVKLD (SEQ ID NO:31) or RQRRNELKRSP (SEQ ID NO:32); the hRNPA1 M9 NLS having the sequence NQSSNFGPMKGGNFGGRSSGPYGGGGQYFAKPRNQGGY (SEQ ID NO:33); the sequence RMRIZFKNKGKDTAELRRRRVEVSVELRKAKKDEQILKRRNV (SEQ ID NO:34) of the IBB domain from importin-alpha; the sequences VSRKRPRP (SEQ ID NO:35) and PPKKARED (SEQ ID NO: 36) of the myoma T protein; the sequence PQPKKKPL (SEQ ID NO:37) of human p53; the sequence SALIKKKKKMAP (SEQ ID NO:38) of mouse c-abl IV; the sequences DRLRR (SEQ ID NO: 39) and PKQKKRK (SEQ ID NO:40) of the influenza virus NS1; the sequence RKLKKKIKKL (SEQ ID NO:41) of the Hepatitis virus delta antigen; the sequence REKKKFLKRR (SEQ ID NO:42) of the mouse Mx1 protein; the sequence KRKGDEVDGVDEVAKKKSKK (SEQ ID NO:43) of the human poly (ADP-ribose) polymerase; and the sequence RKCLQAGMNLEARKTKK (SEQ ID NO:44) of the steroid hormone receptors (human) glucocorticoid. In some cases, a NLS comprises the amino acid sequence PKKKRKV (SEQ ID NO:29), MDSLLMNRRKFLYQFKNVRWAKGRRETYLC (SEQ ID NO: 45), KRTADGSEFESPKKKRKV (SEQ ID NO:46), or KRTADGSEFEPKKKRKV (SEQ ID NO: 47). A Cas13Z polypeptide can include 1, 2, 3, 4, 5, or 6 NLSs. The NLSs can be at the N-terminus, the C-terminus, or both the N-terminus and the C-terminus, of the Cas13Z polypeptide.

### Linkers

In some embodiments, a Cas13Z protein can fused to a fusion partner via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. These linkers can be produced by using synthetic, linker-encoding oligonucleotides to couple the proteins, or can be encoded by a nucleic acid sequence encoding the fusion protein. Peptide linkers with a degree of flexibility can be used. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use.

Examples of linker polypeptides include glycine polymers (G) n, glycine-serine polymers (including, for example, (GS),, GSGGSn (SEQ ID NO:48), GGSGGS, (SEQ ID NO:49), and GGGSn (SEQ ID NO:50), where n is an integer of at least one), glycine-alanine polymers, alanine-serine polymers. Exemplary linkers can comprise amino acid sequences including, but not limited to, GGSG (SEQ ID NO:51), GGSGG (SEQ ID NO:52), GSGSG (SEQ ID NO:53), GSGGG (SEQ ID NO:54), GGGSG (SEQ ID NO:54), GGGGS (SEQ ID NO: 55), and the like. The ordinarily skilled artisan will recognize that design of a peptide conjugated to any desired element can include linkers that are all or partially flexible, such that the linker can include a flexible linker as well as one or more portions that confer less flexible structure.

### Detectable Labels

In some cases, a Cas 13Z polypeptide of the present disclosure comprises a detectable label. Suitable detectable labels and/or moieties that can provide a detectable signal can include, but are not limited to, an enzyme, a radioisotope, a member of a specific binding pair; a fluorophore; a fluorescent protein; a quantum dot; and the like.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilized EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2 (12), mRFP1, pocilloporin, Renilla GFP, Monster GFP, paGFP, Kaede protein and kindling protein, Phycobiliproteins and Phycobiliprotein conjugates including B-Phycocrythrin, R-Phycocrythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

Cas13Z guide nucleic acids

A nucleic acid that binds to a Cas13Z protein, forming a ribonucleoprotein complex (RNP), and that targets the complex to a specific location within a target nucleic acid (e.g., a target RNA) is referred to herein as a “Cas13Z guide RNA” or simply as a “guide RNA.” It is to be understood that in some cases, a hybrid DNA/RNA can be made such that a Cas13Z guide RNA includes DNA bases in addition to RNA bases; the term “Cas13Z guide RNA” is used to encompass such a molecule herein.

A Cas13Z guide RNA can be said to include two segments (regions), a targeting segment and a protein-binding segment. The protein-binding segment is also referred to herein as the “constant region” of the guide RNA. The targeting segment of a Cas13Z guide RNA includes a nucleotide sequence (a guide sequence) that is complementary to (and therefore hybridizes with) a specific sequence (a target site) within a target RNA. The protein-binding segment (or “protein-binding sequence”) interacts with (binds to) a Cas13Z polypeptide. The protein-binding segment of a subject Cas13Z guide RNA can include two complementary stretches of nucleotides that hybridize to one another to form a double stranded RNA duplex (dsRNA duplex). Site-specific binding and/or cleavage of a target RNA can occur at locations (e.g., target sequence of a target locus) determined by base-pairing complementarity between the Cas13Z guide RNA (the guide sequence of the Cas13Z guide RNA) and the target RNA.

A Cas13Z guide RNA and a Cas 13Z protein (e.g., a wild-type Cas13Z protein; a variant Cas13Z protein; a fusion Cas13Z polypeptide; etc.) form a complex (e.g., bind via non-covalent interactions). The Cas13Z guide RNA provides target specificity to the complex by including a targeting segment, which includes a guide sequence (a nucleotide sequence that is complementary to a sequence of a target RNA). The Cas13Z protein of the complex provides the site-specific activity (e.g., cleavage activity provided by the Cas13Z protein and/or an activity provided by the fusion partner in the case of a fusion Cas13Z protein). In other words, the Cas 13Z protein is guided to a target nucleotide sequence (e.g. a target sequence) by virtue of its association with the Cas13Z guide RNA.

The “guide sequence” also referred to as the “targeting sequence” of a Cas13Z guide RNA can be modified so that the Cas13Z guide RNA can target a Cas13Z protein (e.g., a naturally occurring Cas13Z protein, a fusion Cas13Z polypeptide, and the like) to any desired sequence of any desired target RNA with the exception (e.g., as described herein). Thus, for example, a Cas13Z guide RNA can have a guide sequence with complementarity to (e.g., can hybridize to) a sequence in a nucleic acid in a eukaryotic cell, e.g., a viral nucleic acid, a eukaryotic nucleic acid (e.g., a eukaryotic chromosome, chromosomal sequence, a eukaryotic RNA, etc.), and the like.

In some cases, a Cas 13Z guide RNA comprises the structure: 5′-[guide sequence]-[protein-binding segment]-3′.

Guide sequence of a Cas13Z guide RNA

A subject Cas13Z guide RNA includes a guide sequence (i.e., a targeting sequence), which is a nucleotide sequence that is complementary to a sequence (a target site) in a target nucleic acid. In other words, the guide sequence of a Cas13Z guide RNA can interact with a target RNA (double-stranded RNA or single-stranded RNA) in a sequence-specific manner via hybridization (i.e., base pairing). The guide sequence of a Cas13Z guide RNA can be modified (e.g., by genetic engineering)/designed to hybridize to any desired target sequence within a target nucleic acid (e.g., target RNA).

In some cases, the percent complementarity between the guide sequence and the target site of the target nucleic acid is 60% or more (e.g., 65% or more, 70% or more, 75% or more, 80% or more, 85% or more, 90% or more, 95% or more, 97% or more, 98% or more, 99% or more, or 100%). In some cases, the percent complementarity between the guide sequence and the target site of the target nucleic acid is 80% or more (e.g., 85% or more, 90% or more, 95% or more, 97% or more, 98% or more, 99% or more, or 100%). In some cases, the percent complementarity between the guide sequence and the target site of the target nucleic acid is 90% or more (e.g., 95% or more, 97% or more, 98% or more, 99% or more, or 100%). In some cases, the percent complementarity between the guide sequence and the target site of the target nucleic acid is 100%.

In some cases, the percent complementarity between the guide sequence and the target site of the target nucleic acid is 100% over the seven contiguous 3′-most nucleotides of the target site of the target nucleic acid.

In some cases, the percent complementarity between the guide sequence and the target site of the target nucleic acid is 60% or more (e.g., 70% or more, 75% or more, 80% or more, 85% or more, 90% or more, 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 80% or more (e.g., 85% or more, 90% or more, 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 90% or more (e.g., 95% or more, 97% or more, 98% or more, 99% or more, or 100%) over 17 or more (e.g., 18 or more, 19 or more, 20 or more, 21 or more, 22 or more) contiguous nucleotides. In some cases, the percent complementarity between the guide sequence and the target site of the target nucleic acid is 100% over 17 or more (e.g., 18 or more, 19 or more, 20 or more, 21 or more, 22 or more) contiguous nucleotides.

In some cases, the guide sequence has a length in a range of from 17-30 nucleotides (nt)(e.g., from 17-25, 17-22, 17-20, 19-30, 19-25, 19-22, 19-20, 20-30, 20-25, or 20-22 nt). In some cases, the guide sequence has a length in a range of from 17-25 nucleotides (nt)(e.g., from 17-22, 17-20, 19-25, 19-22, 19-20, 20-25, or 20-22 nt). In some cases, the guide sequence has a length of 17 or more nt (e.g., 18 or more, 19 or more, 20 or more, 21 or more, or 22 or more nt; 19 nt, 20 nt, 21 nt, 22 nt, 23 nt, 24 nt, 25 nt, etc.). In some cases, the guide sequence has a length of 19 or more nt (e.g., 20 or more, 21 or more, or 22 or more nt; 19 nt, 20 nt, 21 nt, 22 nt, 23 nt, 24 nt, 25 nt, etc.). In some cases, the guide sequence has a length of 17 nt. In some cases, the guide sequence has a length of 18 nt. In some cases, the guide sequence has a length of 19 nt. In some cases, the guide sequence has a length of 20 nt. In some cases, the guide sequence has a length of 21 nt. In some cases, the guide sequence has a length of 22 nt. In some cases, the guide sequence has a length of 23 nt.

In some cases, the guide sequence (also referred to as a “spacer sequence”) has a length of from 15 to 50 nucleotides (e.g., from 15 nucleotides (nt) to 20 nt, from 20 nt to 25 nt, from 25 nt to 30 nt, from 30 nt to 35 nt, from 35 nt to 40 nt, from 40 nt to 45 nt, or from 45 nt to 50 nt).

Protein-binding segment of a Cas13Z guide RNA

The protein-binding segment (the “constant region”) of a subject Cas13Z guide RNA interacts with a Cas13Z protein. The Cas13Z guide RNA guides the bound Cas13Z protein to a specific nucleotide sequence within target nucleic acid via the above-mentioned guide sequence. The protein-binding segment of a Cas13Z guide RNA can include two stretches of nucleotides that are complementary to one another and hybridize to form a double stranded RNA duplex (dsRNA duplex). Thus, in some cases, the protein-binding segment includes a dsRNA duplex.

In some cases, the dsRNA duplex region includes a range of from 5-25 base pairs (bp)(e.g., from 5-22, 5-20, 5-18, 5-15, 5-12, 5-10, 5-8, 8-25, 8-22, 8-18, 8-15, 8-12, 12-25, 12-22, 12-18, 12-15, 13-25, 13-22, 13-18, 13-15, 14-25, 14-22, 14-18, 14-15, 15-25, 15-22, 15-18, 17-25, 17-22, or 17-18 bp, e.g., 5 bp, 6 bp, 7 bp, 8 bp, 9 bp, 10 bp, etc.). In some cases, the dsRNA duplex region includes a range of from 6-15 base pairs (bp)(e.g., from 6-12, 6-10, or 6-8 bp, e.g., 6 bp, 7 bp, 8 bp, 9 bp, 10 bp, etc.). In some cases, the duplex region includes 5 or more bp (e.g., 6 or more, 7 or more, or 8 or more bp). In some cases, the duplex region includes 6 or more bp (e.g., 7 or more, or 8 or more bp). In some cases, not all nucleotides of the duplex region are paired, and therefore the duplex forming region can include a bulge. The term “bulge” herein is used to mean a stretch of nucleotides (which can be one nucleotide) that do not contribute to a double stranded duplex, but which are surround 5′ and 3′ by nucleotides that do contribute, and as such a bulge is considered part of the duplex region. In some cases, the dsRNA includes 1 or more bulges (e.g., 2 or more, 3 or more, 4 or more bulges). In some cases, the dsRNA duplex includes 2 or more bulges (e.g., 3 or more, 4 or more bulges). In some cases, the dsRNA duplex includes 1-5 bulges (e.g., 1-4, 1-3, 2-5, 2-4, or 2-3 bulges).

Thus, in some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 70%-100% complementarity (e.g., 75%-100%, 80%-10%, 85%-100%, 90%-100%, 95%-100% complementarity) with one another. In some cases, the stretches of nucleotides that hybridize to one another to form the dsRNA duplex have 85%-100% complementarity (e.g., 90%-100%, 95%-100% complementarity) with one another. In some cases, the stretches of nucleotides that hybridize to one another to form the dsRNA duplex have 70%-95% complementarity (e.g., 75%-95%, 80%-95%, 85%-95%, 90%-95% complementarity) with one another.

In other words, in some embodiments, the dsRNA duplex includes two stretches of nucleotides that have 70%-100% complementarity (e.g., 75%-100%, 80%-10%, 85%-100%, 90%-100%, 95%-100% complementarity) with one another. In some cases, the dsRNA duplex includes two stretches of nucleotides that have 85%-100% complementarity (e.g., 90%-100%, 95%-100% complementarity) with one another. In some cases, the dsRNA duplex includes two stretches of nucleotides that have 70%-95% complementarity (e.g., 75%-95%, 80%-95%, 85%-95%, 90%-95% complementarity) with one another.

The duplex region of a subject Cas13Z guide RNA can include one or more (1, 2, 3, 4, 5, etc) mutations relative to a naturally occurring duplex region. For example, in some cases a base pair can be maintained while the nucleotides contributing to the base pair from each segment can be different. In some cases, the duplex region of a subject Cas13Z guide RNA includes more paired bases, less paired bases, a smaller bulge, a larger bulge, fewer bulges, more bulges, or any convenient combination thereof, as compared to a naturally occurring duplex region (of a naturally occurring Cas13Z guide RNA).

In some cases, the protein-binding segment (also referred to in FIG. 1-5 as a “repeat”) has a length of from about 25 nt to about 50 nt (e.g., from 25 nt to 30 nt, from 30 nt to 35 nt, from 35 nt to 40 nt, from 40 nt to 45 nt, or from 45 nt to 50 nt).

Examples of protein-binding segments are provided in FIG. 1-5. For example, a Cas13Z.1 guide RNA can comprise a protein-binding segment comprising the nucleotide sequence: 5′-GCTGGAGCAGCACCCGATTTGCGGTGTAATCACAGC-3′ (SEQ ID NO:2), as depicted in FIG. 1; (e.g., 5′-GCUGGAGCAGCACCCGAUUUGCGGUGUAAUCACAGC-3′ (SEQ ID NO:2)). As another example, a Cas13Z.2 guide RNA can comprise a protein-binding segment comprising the nucleotide sequence: 5′-GCTGGAGCAGCCCTCGATTTGCAGGGTAATCACAGC-3′ (SEQ ID NO:4), as depicted in FIG. 2; (e.g., 5′-GCUGGAGCAGCCCUCGAUUUGCAGGGUAAUCACAGC-3′ (SEQ ID NO: 4)). As another example, a Cas13Z.3 guide RNA can comprise a protein-binding segment comprising the nucleotide sequence: 5′-GCTGGAGCAGCCCTCGATTTGCAGGGTTATCACAGC-3′ (SEQ ID NO: 6), as depicted in FIG. 3; (e.g., 5′-GCUGGAGCAGCCCUCGAUUUGCAGGGUUAUCACAGC-3′ (SEQ ID NO:6)). As another example, a Cas13Z.4 guide RNA can comprise a protein-binding segment comprising the nucleotide sequence: 5′-GCTGAAGCAACCCTGGTTTTGCGGGGTGATTACAGC-3′ (SEQ ID NO:8), as depicted in FIG. 4; (e.g., 5′-GCUGAAGCAACCCUGGUUUUGCGGGGUGAUUACAGC-3′ (SEQ ID NO: 8)). As another example, a Cas13Z.5 guide RNA can comprise a protein-binding segment comprising the nucleotide sequence: 5′-GCTGGAGTAGCCCTCTATTTGAGTGGTGATTACAGC-3′ (SEQ ID NO: 10), as depicted in FIG. 5; (e.g., 5′-GCUGGAGUAGCCCUCUAUUUGAGUGGUGAUUACAGC-3′ (SEQ ID NO:10)).

### Nucleic Acid Modifications

In some cases, a Cas13Z guide RNA comprises one or more modifications, e.g., a base modification, a backbone modification, a sugar modification, etc., to provide the nucleic acid with a new or enhanced feature (e.g., improved stability). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Modified Backbones and Modified Internucleoside Linkages

Examples of suitable modifications include modified nucleic acid backbones and non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a Cas13Z guide RNA comprises one or more phosphorothioate and/or heteroatom internucleoside linkages, in particular —CH₂—NH—O—CH₂—, —CH₂—N(CH₃)—O—CH₂— (known as a methylene(methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids having morpholino backbone structures as described in, e.g., U.S. Pat. No. 5,034,506. For example, in some cases, a Cas13Z guide RNA comprises a 6-membered morpholino ring in place of a ribose ring. In some cases, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A Cas 13Z guide RNA can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound. The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41 (14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonuclcolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

### Modified Sugar Moieties

A Cas13Z guide RNA can also include one or more substituted sugar moieties. Suitable polynucleotides comprise a sugar substituent group selected from: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)ₙO)ₘCH₃, O(CH₂), OCH₃, O(CH₂), NH₂, O(CH₂),CH₃, O(CH₂), ONH₂, and O(CH₂), ON((CH₂)ₙCH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃,ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′—O—CH₂ CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′—O—CH₂—O—CH₂—N(CH₃) 2.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A Cas13Z guide RNA may also include nucleobase (often referred to in the art simply as “base”) modifications or substitutions. As used herein, “unmodified” or “natural” nucleobases include the purine bases adeninc (A) and guaninc (G), and the pyrimidinc bases thyminc (T), cytosinc (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido (5,4-b)(1,4)benzoxazin-2 (3H)-one), phenothiazine cytidine (1H-pyrimido (5,4-b)(1,4)benzothiazin-2 (3H)-one), G-clamps such as a substituted phenoxazine cytidine (e.g. 9-(2-aminoethoxy)-H-pyrimido (5,4-(b)(1,4)benzoxazin-2 (3H)-one), carbazole cytidine (2H-pyrimido (4,5-b) indol-2-one), pyridoindole cytidine (H-pyrido (3′,2′: 4,5) pyrrolo (2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O−6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

### Methods of Modifying a Target RNA

The present disclosure provides a method of modifying a target RNA. The methods generally involve contacting the target RNA with: i) a Cas13Z polypeptide of the present disclosure or a Cas13Z fusion polypeptide of the present disclosure; and ii) a Cas13Z guide RNA, wherein the Cas13Z guide RNA comprises a targeting region comprising a nucleotide sequence that hybridizes with the target RNA.

The target RNA can be a messenger RNA (mRNA), a transfer RNA (tRNA), a ribosomal RNA (rRNA), a microRNA (miRNA), a mitochondrial RNA, and the like. In some cases, the target RNA is an mRNA. In some cases, the target RNA is a mitochondrial RNA.

In some cases, the target RNA is in vitro and is outside of a cell (e.g., in a cell-free system). In some cases, the target RNA is in a living cell, where the living cell is in vitro. In some cases, the target RNA is in a living cell in vivo. In some cases, the target RNA is in vivo and is outside of a cell (e.g., in extracellular fluid). In some cases, the target RNA is in a transcriptome, e.g., a mammalian transcriptome. When the target RNA is in a living cell, the target RNA can be in the nucleus, in the cytoplasm, or both the nucleus and the cytoplasm. In some cases, the target RNA is in a mitochondrion.

In some cases, a subject method for modifying a target RNA comprises modifies translation of a target mRNA. For example, in some cases, a target mRNA is cleaved such that production of a polypeptide encoded by the mRNA is reduced. For example, production of a polypeptide encoded by a target mRNA is reduced by at least 10%, at least 15%, at least 20%, at least 25%, at least 30%, at least 40%, at least 50%, at least 60%, at least 70%, at least 80%, at least 90%, or more than 90%, compared to the level of production of the polypeptide when the target mRNA is not contacted with a Cas13Z polypeptide/Cas 13Z guide RNA complex.

In some cases, a subject method for modifying a target RNA comprises methylating one or more bases in a target RNA. In some cases, a subject method for modifying a target RNA comprises making an N⁶-methyladenosine modification to one or more bases in a target RNA. In some cases, a subject method for modifying a target RNA comprises making a 1-methyladenosine modification to one or more bases in a target RNA. In some cases, a subject method for modifying a target RNA comprises making a 5-hydroxymethylcytidine modification to one or more bases in a target RNA. In some cases, a subject method for modifying a target RNA comprises demethylating one or more methylated bases in a target RNA.

In some cases, a subject method for modifying a target RNA comprises modifying one or more proteins associated with a target RNA.

In some cases, e.g., where the Cas 13Z polypeptide is a Cas13Z fusion polypeptide comprising a methylase or a demethylase as the fusion partner, such that the Cas13Z fusion polypeptide is an RNA methylation editor, the RNA methylation editor can be used for globally changing the epitranscriptome state of a cell, e.g., the methylation state of the expressed transcripts of a cell. In some cases, modifying the methylation state of a target RNA provides for treating a subject having a disease or condition that is caused by a first methylation state of the transcriptome, where the treatment method comprises contacting the diseased cells with an RNA methylation editor as disclosed herein, thereby altering the methylation state of the transcriptome to a second, non-disease associated state.

The present disclosure provides a modified cell comprising a Cas13Z polypeptide of the present disclosure and/or a nucleic acid (e.g., a recombinant expression vector) comprising a nucleotide sequence encoding the Cas13Z polypeptide. The present disclosure provides a modified cell comprising: i) a Cas13Z polypeptide of the present disclosure; and ii) a Cas13Z guide RNA, or a nucleic acid comprising a nucleotide sequence encoding the Cas13Z guide RNA. The present disclosure provides a modified cell comprising: i) a nucleic acid (e.g., a recombinant expression vector) comprising a nucleotide sequence encoding the Cas13Z polypeptide; and ii) a Cas13Z guide RNA, or a nucleic acid comprising a nucleotide sequence encoding the Cas13Z guide RNA. The present disclosure provides a modified cell comprising a Cas13Z fusion polypeptide of the present disclosure and/or a nucleic acid (e.g., a recombinant expression vector) comprising a nucleotide sequence encoding the Cas13Z fusion polypeptide. The present disclosure provides a modified cell comprising: i) a Cas13Z fusion polypeptide of the present disclosure; and ii) a Cas13Z guide RNA, or a nucleic acid comprising a nucleotide sequence encoding the Cas 13Z guide RNA.

A cell that serves as a recipient for a Cas13Z polypeptide or a Cas13Z fusion polypeptide of the present disclosure and/or a nucleic acid comprising a nucleotide sequence encoding a Cas13Z polypeptide or a Cas13Z fusion polypeptide of the present disclosure and/or a Cas13Z guide RNA of the present disclosure, can be any of a variety of cells, including, e.g., in vitro cells; in vivo cells; ex vivo cells; primary cells; cancer cells; animal cells; plant cells; algal cells; fungal cells; bacterial cells; archaeal cells; etc. A cell that serves as a recipient for a Cas13Z polypeptide or a Cas13Z fusion polypeptide of the present disclosure and/or a nucleic acid comprising a nucleotide sequence encoding a Cas13Z polypeptide or a Cas13Z fusion polypeptide of the present disclosure and/or a Cas13Z guide RNA of the present disclosure, is referred to as a “host cell” or a “target cell.” A host cell or a target cell can be a recipient of a Cas13Z system of the present disclosure. A host cell or a target cell can be a recipient of a ribonucleoprotein (RNP) of the present disclosure, where the RNP comprises: i) a Cas13Z polypeptide of the present disclosure; and ii) a Cas13Z guide RNA. A host cell or a target cell can be a recipient of a single component of a system of the present disclosure.

Non-limiting examples of cells (target cells) include: a prokaryotic cell, eukaryotic cell, a bacterial cell, an archaeal cell, a cell of a single-cell eukaryotic organism, a protozoa cell, a cell from a plant (e.g., cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, angiosperms, ferns, clubmosses, hornworts, liverworts, mosses, dicotyledons, monocotyledons, etc.), an algal cell, (e.g., Botryococcus braunii, Chlamydomonas reinhardtii, Nannochloropsis gaditana, Chlorella pyrenoidosa, Sargassum patens, C. agardh, and the like), seaweeds (e.g. kelp) a fungal cell (e.g., a yeast cell, a cell from a mushroom), an animal cell, a cell from an invertebrate animal (e.g., fruit fly, cnidarian, echinoderm, nematode, etc.), a cell from a vertebrate animal (e.g., fish, amphibian, reptile, bird, mammal), a cell from a mammal (e.g., an ungulate (e.g., a pig, a cow, a goat, a sheep); a rodent (e.g., a rat, a mouse); a non-human primate; a human; a feline (e.g., a cat); a canine (e.g., a dog); etc.), and the like. In some cases, the cell is a cell that does not originate from a natural organism (e.g., the cell can be a synthetically made cell; also referred to as an artificial cell).

A cell can be an in vitro cell (e.g., established cultured cell line). A cell can be an ex vivo cell (cultured cell from an individual). A cell can be an in vivo cell (e.g., a cell in an individual). A cell can be an isolated cell. A cell can be a cell inside of an organism. A cell can be an organism. A cell can be a cell in a cell culture (e.g., in vitro cell culture). A cell can be one of a collection of cells. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a mammalian cell or derived from a mammalian cell. A cell can be a rodent cell or derived from a rodent cell. A cell can be a human cell or derived from a human cell. A cell can be a microbe cell or derived from a microbe cell. A cell can be a fungi cell or derived from a fungi cell. A cell can be an insect cell. A cell can be an arthropod cell. A cell can be a protozoan cell. A cell can be a helminth cell.

Suitable cells include a stem cell (e.g. an embryonic stem (ES) cell, an induced pluripotent stem (iPS) cell; a germ cell (e.g., an oocyte, a sperm, an oogonia, a spermatogonia, etc.); a somatic cell, e.g. a fibroblast, an oligodendrocyte, a glial cell, a hematopoietic cell, a neuron, a muscle cell, a bone cell, a hepatocyte, a pancreatic cell, etc.

Suitable cells include human embryonic stem cells, fetal cardiomyocytes, myofibroblasts, mesenchymal stem cells, cardiomyocytes, adipocytes, totipotent cells, pluripotent cells, blood stem cells, myoblasts, adult stem cells, bone marrow cells, mesenchymal cells, embryonic stem cells, parenchymal cells, epithelial cells, endothelial cells, mesothelial cells, fibroblasts, osteoblasts, chondrocytes, exogenous cells, endogenous cells, stem cells, hematopoietic stem cells, bone-marrow derived progenitor cells, myocardial cells, skeletal cells, fetal cells, undifferentiated cells, multi-potent progenitor cells, unipotent progenitor cells, monocytes, cardiac myoblasts, skeletal myoblasts, macrophages, capillary endothelial cells, xenogeneic cells, allogeneic cells, and post-natal stem cells.

In some cases, the cell is an immune cell, a neuron, an epithelial cell, and endothelial cell, or a stem cell. In some cases, the immune cell is a T cell, a B cell, a monocyte, a natural killer cell, a dendritic cell, or a macrophage. In some cases, the immune cell is a cytotoxic T cell. In some cases, the immune cell is a helper T cell. In some cases, the immune cell is a regulatory T cell (Treg).

In some cases, the cell is a stem cell. Stem cells include adult stem cells. Adult stem cells are also referred to as somatic stem cells.

Adult stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells; hematopoietic stem cells; epithelial stem cells; neural stem cells; mesenchymal stem cells; mammary stem cells; intestinal stem cells; mesodermal stem cells; endothelial stem cells; olfactory stem cells; neural crest stem cells; and the like.

Stem cells of interest include mammalian stem cells, where the term “mammalian” refers to any animal classified as a mammal, including humans; non-human primates; domestic and farm animals; and zoo, laboratory, sports, or pet animals, such as dogs, horses, cats, cows, mice, rats, rabbits, etc. In some cases, the stem cell is a human stem cell. In some cases, the stem cell is a rodent (e.g., a mouse; a rat) stem cell. In some cases, the stem cell is a non-human primate stem cell.

Stem cells can express one or more stem cell markers, e.g., SOX9, KRT19, KRT7, LGR5, CA9, FXYD2, CDH6, CLDN18, TSPAN8, BPIFB1, OLFM4, CDH17, and PPARGC1A.

In some cases, the stem cell is a hematopoietic stem cell (HSC). HSCs are mesoderm-derived cells that can be isolated from bone marrow, blood, cord blood, fetal liver and yolk sac. HSCs are characterized as CD34+ and CD3−. HSCs can repopulate the erythroid, neutrophil-macrophage, megakaryocyte and lymphoid hematopoietic cell lineages in vivo. In vitro, HSCs can be induced to undergo at least some self-renewing cell divisions and can be induced to differentiate to the same lineages as is seen in vivo. As such, HSCs can be induced to differentiate into one or more of erythroid cells, megakaryocytes, neutrophils, macrophages, and lymphoid cells.

In other instances, the stem cell is a neural stem cell (NSC). Neural stem cells (NSCs) can differentiate into neurons, and glia (including oligodendrocytes, and astrocytes). A neural stem cell is a multipotent stem cell which is capable of multiple divisions, and under specific conditions can produce daughter cells which are neural stem cells, or neural progenitor cells that can be neuroblasts or glioblasts, e.g., cells committed to become one or more types of neurons and glial cells respectively. Methods of obtaining NSCs are known in the art.

In other instances, the stem cell is a mesenchymal stem cell (MSC). MSCs originally derived from the embryonal mesoderm and isolated from adult bone marrow, can differentiate to form muscle, bone, cartilage, fat, marrow stroma, and tendon. Methods of isolating MSC are known in the art; and any known method can be used to obtain MSC. See, e.g., U.S. Pat. No. 5,736,396, which describes isolation of human MSC.

A cell is in some cases a plant cell. A plant cell can be a cell of a monocotyledon. A cell can be a cell of a dicotyledon.

In some cases, the cell is a plant cell. For example, the cell can be a cell of a major agricultural plant, e.g., Barley, Beans (Dry Edible), Canola, Corn, Cotton (Pima), Cotton (Upland), Flaxseed, Hay (Alfalfa), Hay (Non-Alfalfa), Oats, Peanuts, Ricc, Sorghum, Soybeans, Sugarbects, Sugarcane, Sunflowers (Oil), Sunflowers (Non-Oil), Sweet Potatoes, Tobacco (Burley), Tobacco (Flue-cured), Tomatoes, Wheat (Durum), Wheat (Spring), Wheat (Winter), and the like. As another example, the cell is a cell of a vegetable crops which include but are not limited to, e.g., alfalfa sprouts, aloe leaves, arrow root, arrowhead, artichokes, asparagus, bamboo shoots, banana flowers, bean sprouts, beans, beet tops, beets, bittermelon, bok choy, broccoli, broccoli rabe (rappini), brussels sprouts, cabbage, cabbage sprouts, cactus leaf (nopales), calabaza, cardoon, carrots, cauliflower, celery, chayote, chinese artichoke (crosnes), chinese cabbage, chinese celery, chinese chives, choy sum, chrysanthemum leaves (tung ho), collard greens, corn stalks, corn-sweet, cucumbers, daikon, dandelion greens, dasheen, dau mue (pea tips), donqua (winter melon), eggplant, endive, escarole, fiddle head ferns, field cress, frisee, gai choy (chinese mustard), gailon, galanga (siam, thai ginger), garlic, ginger root, gobo, greens, hanover salad greens, huauzontle, jerusalem artichokes, jicama, kale greens, kohlrabi, lamb's quarters (quilete), lettuce (bibb), lettuce (boston), lettuce (boston red), lettuce (green leaf), lettuce (iceberg), lettuce (lolla rossa), lettuce (oak leaf-green), lettuce (oak leaf-red), lettuce (processed), lettuce (red leaf), lettuce (romaine), lettuce (ruby romaine), lettuce (russian red mustard), linkok, lo bok, long beans, lotus root, mache, maguey (agave) leaves, malanga, mesculin mix, mizuna, moap (smooth luffa), moo, moqua (fuzzy squash), mushrooms, mustard, nagaimo, okra, ong choy, onions green, opo (long squash), ornamental corn, ornamental gourds, parsley, parsnips, peas, peppers (bell type), peppers, pumpkins, radicchio, radish sprouts, radishes, rape greens, rape greens, rhubarb, romaine (baby red), rutabagas, salicornia (sea bean), sinqua (angled/ridged luffa), spinach, squash, straw bales, sugarcane, sweet potatoes, swiss chard, tamarindo, taro, taro leaf, taro shoots, tatsoi, tepeguaje (guaje), tindora, tomatillos, tomatoes, tomatoes (cherry), tomatoes (grape type), tomatoes (plum type), tumeric, turnip tops greens, turnips, water chestnuts, yampi, yams (names), yu choy, yuca (cassava), and the like.

A cell is in some cases an arthropod cell. For example, the cell can be a cell of a sub-order, a family, a sub-family, a group, a sub-group, or a species of, e.g., Chelicerata, Myriapodia, Hexipodia, Arachnida, Insecta, Archaeognatha, Thysanura, Palaeoptera, Ephemeroptera, Odonata, Anisoptera, Zygoptera, Neoptera, Exopterygota, Plecoptera, Embioptera, Orthoptera, Zoraptera, Dermaptera, Dictyoptera, Notoptera, Grylloblattidae, Mantophasmatidae, Phasmatodea, Blattaria, Isoptera, Mantodea, Parapneuroptera, Psocoptera, Thysanoptera, Phthiraptera, Hemiptera, Endopterygota or Holometabola, Hymenoptera, Coleoptera, Strepsiptera, Raphidioptera, Megaloptera, Neuroptera, Mecoptera, Siphonaptera, Diptera, Trichoptera, or Lepidoptera.

A cell is in some cases an insect cell. For example, in some cases, the cell is a cell of a mosquito, a grasshopper, a true bug, a fly, a flea, a bee, a wasp, an ant, a louse, a moth, or a beetle.

### Compositions and Systems

The present disclosure provides compositions and systems comprising one or more of: i) a Cas13Z polypeptide of the present disclosure; ii) a nucleic acid comprising a nucleotide sequence encoding a Cas13Z polypeptide of the present disclosure; iii) a Cas13Z fusion polypeptide of the present disclosure; iv) a nucleic acid comprising a nucleotide sequence encoding a Cas13Z fusion polypeptide of the present disclosure; v) a Cas13Z guide RNA of the present disclosure; vi) a nucleic acid comprising a nucleotide sequence encoding a Cas 13Z guide RNA of the present disclosure; vii) a nucleic acid comprising a nucleotide sequence encoding a Cas13Z polypeptide of the present disclosure and a nucleotide sequence encoding a Cas13Z guide RNA of the present disclosure; and viii) a nucleic acid comprising a nucleotide sequence encoding a Cas13Z fusion polypeptide of the present disclosure and a nucleotide sequence encoding a Cas13Z guide RNA of the present disclosure.

The present disclosure provides a composition comprising a Cas13Z polypeptide of the present disclosure. In some cases, a composition of the present disclosure comprises one or more of: a) a lipid; b) a buffer; c) a nuclease inhibitor; d) a protease inhibitor; e) one or more Cas13Z guide RNAs, or one or more nucleic acids comprising nucleotide sequences encoding the one or more Cas 13Z guide RNAs. The present disclosure provides a composition comprising a ribonucleoprotein (RNP) complex, where the RNP complex comprises a Cas13Z polypeptide of the present disclosure and a guide RNA. The present disclosure provides a composition comprising a nucleic acid comprising a nucleotide sequence encoding a Cas 13Z polypeptide of the present disclosure, or comprising a recombinant expression vector comprising the nucleic acid.

The present disclosure provides a system comprising a Cas13Z polypeptide of the present disclosure. A system of the present disclosure can comprise: a) a Cas13Z polypeptide of the present disclosure and a Cas13Z guide RNA; b) a Cas13Z fusion polypeptide of the present disclosure and a Cas13Z guide RNA; c) an mRNA encoding a Cas13Z of the present disclosure; and a Cas13Z guide RNA; d) an mRNA encoding a Cas13Z fusion polypeptide of the present disclosure; and a Cas13Z guide RNA; e) a recombinant expression vector comprising a nucleotide sequence encoding a Cas13Z s polypeptide of the present disclosure and a nucleotide sequence encoding a Cas13Z guide RNA; f) a recombinant expression vector comprising a nucleotide sequence encoding a Cas13Z fusion polypeptide of the present disclosure and a nucleotide sequence encoding a Cas13Z guide RNA; g) a first recombinant expression vector comprising a nucleotide sequence encoding a Cas13Z polypeptide of the present disclosure and a second recombinant expression vector comprising a nucleotide sequence encoding a Cas13Z guide RNA; h) a first recombinant expression vector comprising a nucleotide sequence encoding a Cas 13Z polypeptide of the present disclosure, and a second recombinant expression vector comprising a nucleotide sequence encoding a Cas13Z guide RNA; i) a recombinant expression vector comprising a nucleotide sequence encoding a Cas 13Z polypeptide of the present disclosure, a nucleotide sequence encoding a first Cas13Z guide RNA, and a nucleotide sequence encoding a second Cas13Z guide RNA; or j) a recombinant expression vector comprising a nucleotide sequence encoding a Cas13Z fusion polypeptide of the present disclosure, a nucleotide sequence encoding a first Cas13Z guide RNA, and a nucleotide sequence encoding a second Cas13Z guide RNA; or some variation of one of (a) through (j).

### Nucleic Acids, Recombinant Expression Vectors, and Host Cells

The present disclosure provides one or more nucleic acids comprising one or more of: a nucleotide sequence encoding Cas13Z polypeptide of the present disclosure, a Cas13Z guide RNA, and a nucleotide sequence encoding a Cas13Z guide RNA. The present disclosure provides a nucleic acid comprising a nucleotide sequence encoding a Cas13Z polypeptide of the present disclosure. The present disclosure provides a nucleic acid comprising a nucleotide sequence encoding a fusion polypeptide comprising: a) a Cas13Z polypeptide of the present disclosure; and b) one or more heterologous polypeptides (one or more fusion partners). The present disclosure provides a recombinant expression vector that comprises a nucleotide sequence encoding a Cas13Z polypeptide of the present disclosure.

The present disclosure provides a recombinant expression vector that comprises a nucleotide sequence encoding a Cas13Z fusion polypeptide of the present disclosure. The present disclosure provides a recombinant expression vector that comprises: a) a nucleotide sequence encoding a Cas13Z polypeptide of the present disclosure; and b) a nucleotide sequence encoding a Cas13Z guide RNA(s). The present disclosure provides a recombinant expression vector that comprises: a) a nucleotide sequence encoding a Cas13Z fusion polypeptide of the present disclosure; and b) a nucleotide sequence encoding a Cas13Z guide RNA. In some cases, the nucleotide sequence encoding the Cas13Z polypeptide of the present disclosure and/or the nucleotide sequence encoding the Cas13Z guide RNA and/or the nucleotide sequence encoding the Cas13Z fusion polypeptide is operably linked to a promoter that is operable in a cell type of choice (e.g., a prokaryotic cell, a eukaryotic cell, an archaeal cell, a plant cell, an animal cell, a mammalian cell, a primate cell, a rodent cell, a human cell, etc.). Various nucleic acid and expression vectors are described below in the context of a Cas13Z polypeptide of the present disclosure; these descriptions apply equally to a Cas13Z fusion polypeptide of the present disclosure.

In some cases, a nucleotide sequence encoding a Cas13Z polypeptide of the present disclosure, or a Cas13Z fusion polypeptide of the present disclosure, is codon optimized. This type of optimization can entail a mutation of a Cas 13Z polypeptide-encoding nucleotide sequence to mimic the codon preferences of the intended host organism or cell while encoding the same protein. Thus, the codons can be changed, but the encoded protein remains unchanged. For example, if the intended target cell was a human cell, a human codon-optimized Cas13Z polypeptide-encoding nucleotide sequence could be used. As another non-limiting example, if the intended host cell were a mouse cell, then a mouse codon-optimized Cas13Z polypeptide-encoding nucleotide sequence could be generated. As another non-limiting example, if the intended host cell were a plant cell, then a plant codon-optimized Cas 13Z polypeptide-encoding nucleotide sequence could be generated. As another non-limiting example, if the intended host cell were an insect cell, then an insect codon-optimized Cas13Z polypeptide-encoding nucleotide sequence could be generated.

The present disclosure provides one or more recombinant expression vectors that include (in separate recombinant expression vectors in some cases, and in the same recombinant expression vector in some cases): (i) a nucleotide sequence that encodes a Cas13Z guide RNA that hybridizes to a target sequence a target RNA (e.g., operably linked to a promoter that is operable in a target cell such as a eukaryotic cell); and (ii) a nucleotide sequence encoding a Cas13Z polypeptide of the present disclosure (e.g., operably linked to a promoter that is operable in a target cell such as a eukaryotic cell).

Suitable expression vectors include viral expression vectors (e.g. viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (AAV)(see, e.g., Ali et al., Hum Gene Ther 9:81 86, 1998, Flannery et al., PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al., Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like. In some cases, a recombinant expression vector of the present disclosure is a recombinant adeno-associated virus (AAV) vector. In some cases, a recombinant expression vector of the present disclosure is a recombinant lentivirus vector. In some cases, a recombinant expression vector of the present disclosure is a recombinant retroviral vector.

Depending on the host/vector system utilized, any of a number of suitable transcription and translation control elements, including constitutive and inducible promoters, transcription enhancer elements, transcription terminators, etc. may be used in the expression vector.

In some cases, a nucleotide sequence encoding a Cas13Z guide RNA is operably linked to a control element, e.g., a transcriptional control element, such as a promoter. In some embodiments, a nucleotide sequence encoding a Cas13Z polypeptide of the present disclosure or a fusion polypeptide of the present disclosure is operably linked to a control element, e.g., a transcriptional control element, such as a promoter.

The transcriptional control element can be a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population.

Non-limiting examples of eukaryotic promoters (promoters functional in a eukaryotic cell) include EF1α, those from cytomegalovirus (CMV) immediate early, herpes simplex virus (HSV) thymidine kinase, early and late SV40, long terminal repeats (LTRs) from retrovirus, and mouse metallothionein-I. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6xHis tag, hemagglutinin tag, fluorescent protein, etc.) that can be fused to Cas13Z polypeptide of the present disclosure, thus resulting in a fusion polypeptide.

In some cases, a nucleotide sequence encoding a Cas13Z guide RNA and/or a Cas13Z polypeptide of the present disclosure, or a fusion polypeptide of the present disclosure, is operably linked to an inducible promoter. In some cases, a nucleotide sequence encoding a Cas13Z guide RNA and/or a Cas13Z polypeptide of the present disclosure is operably linked to a constitutive promoter.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6) (Miyagishi et al., Nature Biotechnology 20, 497-500 (2002)), an enhanced U6 promoter (e.g., Xia et al., Nucleic Acids Res. 2003 Sep. 1;31 (17)), a human H1 promoter (H1), and the like.

In some cases, a nucleotide sequence encoding a Cas13Z guide RNA is operably linked to (under the control of) a promoter operable in a eukaryotic cell (e.g., a U6 promoter, an enhanced U6 promoter, an H1 promoter, and the like). As would be understood by one of ordinary skill in the art, when expressing an RNA (e.g., a guide RNA) from a nucleic acid (e.g., an expression vector) using a U6 promoter (e.g., in a eukaryotic cell), or another PolIII promoter, the RNA may need to be mutated if there are several Ts in a row (coding for Us in the RNA). This is because a string of Ts (e.g., 5 Ts) in DNA can act as a terminator for polymerase III (Pol III). Thus, in order to ensure transcription of a guide RNA in a eukaryotic cell it may sometimes be necessary to modify the sequence encoding the guide RNA to eliminate runs of Ts. In some cases, a nucleotide sequence encoding a Cas 13Z polypeptide of the present disclosure is operably linked to a promoter operable in a eukaryotic cell (e.g., a CMV promoter, an EF1α promoter, an estrogen receptor-regulated promoter, and the like).

Examples of inducible promoters include, but are not limited toT7 RNA polymerase promoter, T3 RNA polymerase promoter, Isopropyl-bcta-D-thiogalactopyranoside (IPTG)-regulated promoter, lactose induced promoter, heat shock promoter, Tetracycline-regulated promoter, Steroid-regulated promoter, Metal-regulated promoter, estrogen receptor-regulated promoter, etc. Inducible promoters can therefore be regulated by molecules including, but not limited to, doxycycline; estrogen and/or an estrogen analog; IPTG; etc.

Inducible promoters suitable for use include any inducible promoter described herein or known to one of ordinary skill in the art. Examples of inducible promoters include, without limitation, chemically/biochemically-regulated and physically-regulated promoters such as alcohol-regulated promoters, tetracycline-regulated promoters (e.g., anhydrotetracycline (aTc)-responsive promoters and other tetracycline-responsive promoter systems, which include a tetracycline repressor protein (tetR), a tetracycline operator sequence (tetO) and a tetracycline transactivator fusion protein (tTA)), steroid-regulated promoters (e.g., promoters based on the rat glucocorticoid receptor, human estrogen receptor, moth ecdysone receptors, and promoters from the steroid/retinoid/thyroid receptor superfamily), metal-regulated promoters (e.g., promoters derived from metallothionein (proteins that bind and sequester metal ions) genes from yeast, mouse and human), pathogenesis-regulated promoters (e.g., induced by salicylic acid, ethylene or benzothiadiazole (BTH)), temperature/heat-inducible promoters (e.g., heat shock promoters), and light-regulated promoters (e.g., light responsive promoters from plant cells).

In some cases, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used as long as the promoter is functional in the targeted host cell (e.g., eukaryotic cell; prokaryotic cell).

In some cases, the promoter is a reversible promoter. Suitable reversible promoters, including reversible inducible promoters are known in the art. Such reversible promoters may be isolated and derived from many organisms, e.g., eukaryotes and prokaryotes. Modification of reversible promoters derived from a first organism for use in a second organism, e.g., a first prokaryote and a second a eukaryote, a first eukaryote and a second a prokaryote, etc., is well known in the art. Such reversible promoters, and systems based on such reversible promoters but also comprising additional control proteins, include, but are not limited to, alcohol regulated promoters (e.g., alcohol dehydrogenase I(alcA) gene promoter, promoters responsive to alcohol transactivator proteins (AlcR), etc.), tetracycline regulated promoters, (e.g., promoter systems including TetActivators, TetON, TetOFF, etc.), steroid regulated promoters (e.g., rat glucocorticoid receptor promoter systems, human estrogen receptor promoter systems, retinoid promoter systems, thyroid promoter systems, ecdysone promoter systems, mifepristone promoter systems, etc.), metal regulated promoters (e.g., metallothionein promoter systems, etc.), pathogenesis-related regulated promoters (e.g., salicylic acid regulated promoters, ethylene regulated promoters, benzothiadiazole regulated promoters, etc.), temperature regulated promoters (e.g., heat shock inducible promoters (e.g., HSP-70, HSP-90, soybean heat shock promoter, etc.), light regulated promoters, synthetic inducible promoters, and the like.

Methods of introducing a nucleic acid (e.g., a nucleic acid comprising a donor polynucleotide sequence, one or more nucleic acids encoding a Cas13Z polypeptide of the present disclosure (or a fusion polypeptide of the present disclosure) and/or a Cas13Z guide RNA, and the like) into a host cell are known in the art, and any convenient method can be used to introduce a nucleic acid (e.g., an expression construct) into a cell. Suitable methods include e.g., viral infection, transfection, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct microinjection, nanoparticle-mediated nucleic acid delivery, and the like.

Introducing the recombinant expression vector into cells can occur in any culture media and under any culture conditions that promote the survival of the cells. Introducing the recombinant expression vector into a target cell can be carried out in vivo or ex vivo. Introducing the recombinant expression vector into a target cell can be carried out in vitro.

In some embodiments, a Cas 13Z polypeptide of the present disclosure can be provided as RNA. The RNA can be provided by direct chemical synthesis or may be transcribed in vitro from a DNA (e.g., encoding the Cas13Z polypeptide). Once synthesized, the RNA may be introduced into a cell by any of the well-known techniques for introducing nucleic acids into cells (e.g., microinjection, electroporation, transfection, etc.).

Nucleic acids may be provided to the cells using well-developed transfection techniques; see, e.g. Angel and Yanik (2010) PLOS ONE 5 (7): e11756, and the commercially available TransMessenger® reagents from Qiagen, Stemfect™ RNA Transfection Kit from Stemgent, and TransIT®-mRNA Transfection Kit from Mirus Bio LLC. See also Beumer et al. (2008) PNAS 105 (50): 19821-19826.

Vectors may be provided directly to a target host cell. In other words, the cells are contacted with vectors comprising the subject nucleic acids (e.g., recombinant expression vectors having the donor template sequence and encoding a Cas13Z guide RNA; recombinant expression vectors encoding a Cas13Z polypeptide of the present disclosure (or a fusion polypeptide of the present disclosure); etc.) such that the vectors are taken up by the cells. Methods for contacting cells with nucleic acid vectors that are plasmids, include electroporation, calcium chloride transfection, microinjection, and lipofection are well known in the art. For viral vector delivery, cells can be contacted with viral particles comprising the subject viral expression vectors.

Retroviruses, for example, lentiviruses, are suitable for use in methods of the present disclosure. Commonly used retroviral vectors are “defective”, i.e. unable to produce viral proteins required for productive infection. Rather, replication of the vector requires growth in a packaging cell line. To generate viral particles comprising nucleic acids of interest, the retroviral nucleic acids comprising the nucleic acid are packaged into viral capsids by a packaging cell line. Different packaging cell lines provide a different envelope protein (ecotropic, amphotropic or xenotropic) to be incorporated into the capsid, this envelope protein determining the specificity of the viral particle for the cells (ecotropic for murine and rat; amphotropic for most mammalian cell types including human, dog and mouse; and xenotropic for most mammalian cell types except murine cells). The appropriate packaging cell line may be used to ensure that the cells are targeted by the packaged viral particles. Methods of introducing subject vector expression vectors into packaging cell lines and of collecting the viral particles that are generated by the packaging lines are well known in the art. Nucleic acids can also introduced by direct micro-injection (e.g., injection of RNA).

Vectors used for providing the nucleic acids encoding Cas13Z guide RNA and/or a Cas13Z polypeptide of the present disclosure (or a fusion polypeptide of the present disclosure) to a target host cell can include suitable promoters for driving the expression, that is, transcriptional activation, of the nucleic acid of interest. In other words, in some cases, the nucleic acid of interest will be operably linked to a promoter. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by 10 fold, by 100 fold, more usually by 1000 fold. In addition, vectors used for providing a nucleic acid encoding a Cas13Z guide RNA and/or a Cas13Z polypeptide of the present disclosure to a cell may include nucleic acid sequences that encode for selectable markers in the target cells, so as to identify cells that have taken up the Cas13Z guide RNA and/or Cas13Z polypeptide.

A nucleic acid comprising a nucleotide sequence encoding a Cas 13Z polypeptide of the present disclosure, or a fusion polypeptide of the present disclosure (a fusion polypeptide comprising: a) a Cas13Z polypeptide of the present disclosure; and b) one or more heterologous polypeptides), is in some cases an RNA. Thus, a fusion protein of the present disclosure can be introduced into cells as RNA. Methods of introducing RNA into cells are known in the art and may include, for example, direct injection, transfection, or any other method used for the introduction of DNA. A Cas13Z polypeptide of the present disclosure may instead be provided to cells as a polypeptide. Such a polypeptide may optionally be fused to a polypeptide domain that increases solubility of the product. The domain may be linked to the polypeptide through a defined protease cleavage site, e.g. a tobacco etch virus (TEV) proteolytically cleavable peptide, which is cleaved by TEV protease. The linker may also include one or more flexible sequences, e.g. from 1 to 10 glycine residues. In some embodiments, the cleavage of the fusion protein is performed in a buffer that maintains solubility of the product, e.g. in the presence of from 0.5 to 2 M urea, in the presence of polypeptides and/or polynucleotides that increase solubility, and the like. Domains of interest include endosomolytic domains, e.g. influenza HA domain; and other polypeptides that aid in production, e.g. IF2 domain, GST domain, GRPE domain, and the like. The polypeptide may be formulated for improved stability. For example, the peptides may be PEGylated, where the polyethyleneoxy group provides for enhanced lifetime in the blood stream.

Additionally, or alternatively, a Cas13Z polypeptide of the present disclosure may be fused to a polypeptide permeant domain to promote uptake by the cell. A number of permeant domains are known in the art and may be used in the non-integrating polypeptides of the present disclosure, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 61). As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4 (2): 87-9 and 446; and Wender et al. (2000) Proc. Natl. Acad. Sci. U.S.A 2000 Nov. 21; 97 (24): 13003-8; published U.S. patent applications Ser. No. 20/030,220334; 20030083256; 20030032593; and 20030022831, herein specifically incorporated by reference for the teachings of translocation peptides and peptoids). The nona-arginine (R9) sequence can be used. The site at which the fusion is made may be selected in order to optimize the biological activity, secretion or binding characteristics of the polypeptide. The optimal site will be determined by routine experimentation.

A Cas13Z polypeptide of the present disclosure may be produced in vitro or by eukaryotic cells or by prokaryotic cells, and it may be further processed by unfolding, e.g. heat denaturation, dithiothreitol reduction, etc. and may be further refolded, using methods known in the art.

Modifications of interest that do not alter primary sequence include chemical derivatization of polypeptides, e.g., acylation, acctylation, carboxylation, amidation, etc. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also encompassed are sequences that have phosphorylated amino acid residues, e.g. phosphotyrosine, phosphoserine, or phosphothreonine.

Also suitable for inclusion in embodiments of the present disclosure are nucleic acids (e.g., encoding a Cas13Z guide RNA, encoding a fusion protein of the present disclosure, etc.) and proteins (e.g., a Cas13Z polypeptide of the present disclosure; a fusion protein of the present disclosure) that have been modified using ordinary molecular biological techniques and synthetic chemistry so as to improve their resistance to proteolytic degradation, to change the target sequence specificity, to optimize solubility properties, to alter protein activity (e.g., transcription modulatory activity, enzymatic activity, etc.) or to render them more suitable. Analogs of such polypeptides include those containing residues other than naturally occurring L-amino acids, e.g. D-amino acids or non-naturally occurring synthetic amino acids. D-amino acids may be substituted for some or all of the amino acid residues.

A Cas 13Z polypeptide of the present disclosure may be prepared by in vitro synthesis, using conventional methods as known in the art. Various commercial synthetic apparatuses are available, for example, automated synthesizers by Applied Biosystems, Inc., Beckman, etc. By using synthesizers, naturally occurring amino acids may be substituted with unnatural amino acids. The particular sequence and the manner of preparation will be determined by convenience, economics, purity required, and the like.

If desired, various groups may be introduced into the peptide during synthesis or during expression, which allow for linking to other molecules or to a surface. Thus, cysteines can be used to make thioethers, histidines for linking to a metal ion complex, carboxyl groups for forming amides or esters, amino groups for forming amides, and the like.

A Cas13Z polypeptide of the present disclosure may also be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 20% or more by weight of the desired product, more usually 75% or more by weight, preferably 95% or more by weight, and for therapeutic purposes, usually 99.5% or more by weight, in relation to contaminants related to the method of preparation of the product and its purification. Usually, the percentages will be based upon total protein. Thus, in some cases, a Cas 13Z polypeptide of the present disclosure, or a fusion polypeptide of the present disclosure, is at least 80% pure, at least 85% pure, at least 90% pure, at least 95% pure, at least 98% pure, or at least 99% pure (e.g., free of contaminants, Cas13Z proteins or other macromolecules, etc.).

### Detection Methods

The present disclosure provides a method of detecting an RNA in a sample. In some cases, the sample is a cell-free sample. In some cases, the sample comprises cells. In some cases, the sample comprises a cell lysate.

Provided are compositions and methods for detecting a target RNA, where the methods include (i) contacting a sample having a plurality of RNAs with (a) a Cas13Z guide RNA that hybridizes with the target RNA, and (b) a Cas13Z protein that cleaves RNAs (e.g., cleaves non-target RNAs in a sequence non-specific manner, where a non-target RNA can comprise a detectable label) present in the sample; and (ii) measuring a detectable signal produced by the cleavage. Once a subject Cas 13Z protein is activated by a Cas13Z guide RNA, which occurs when the sample includes a target RNA to which the guide RNA hybridizes (i.e., the sample includes the target RNA), the Cas13Z protein is activated and functions as an endoribonuclease that non-specifically cleaves RNAs (including non-target RNAs) present in the sample. Thus, when the target RNA is present in the sample (e.g., in some cases above a threshold amount), the result is cleavage of RNA (including non-target RNA) in the sample, which can be detected using any convenient detection method (e.g., using a labeled detector RNA). The contacting step is generally carried out in a composition comprising divalent metal ions. The contacting step can be carried out in an acellular environment, e.g., outside of a cell. The contacting step can be carried out inside a cell. The contacting step can be carried out in a cell in vitro. The contacting step can be carried out in a cell ex vivo. The contacting step can be carried out in a cell in vivo. In some cases, the Cas13Z guide RNA is provided as RNA; and the Cas13Z protein is provided as protein per se. In some cases, the Cas13Z guide RNA is provided as DNA encoding the guide RNA; and the Cas13Z protein is provided as protein per se. In some cases, the Cas13Z guide RNA is provided as RNA; and the Cas13Z protein is provided as RNA encoding the Cas13Z protein. In some cases, the Cas13Z guide RNA is provided as DNA encoding the guide RNA; and Cas13Z protein is provided as RNA encoding the Cas 13Z protein. In some cases, the Cas13Z guide RNA is provided as RNA; and the Cas13Z protein is provided as DNA comprising a nucleotide sequence encoding the Cas13Z protein. In some cases, the Cas13Z guide RNA is provided as DNA encoding the guide RNA; and the Cas13Z protein is provided as DNA comprising a nucleotide sequence encoding the Cas13Z protein. In some cases, a method of the present disclosure provides for substantially simultaneous detection of two different target RNAs (a first single-stranded target RNA and a second single-stranded target RNA) in a sample.

In some cases (e.g., when contacting with a Cas13Z guide RNA and a Cas13Z protein), the sample is contacted for 2 hours or less (e.g., 1.5 hours or less, 1 hour or less, 40 minutes or less, 30 minutes or less, 20 minutes or less, 10 minutes or less, or 5 minutes or less, or 1 minute or less) prior to the measuring step. For example, in some cases the sample is contacted for 40 minutes or less prior to the measuring step. In some cases, the sample is contacted for 20 minutes or less prior to the measuring step.

In some cases, the sample is contacted for 10 minutes or less prior to the measuring step. In some cases, the sample is contacted for 5 minutes or less prior to the measuring step. In some cases, the sample is contacted for 1 minute or less prior to the measuring step. In some cases, the sample is contacted for from 50 seconds to 60 seconds prior to the measuring step. In some cases, the sample is contacted for from 40 seconds to 50 seconds prior to the measuring step. In some cases, the sample is contacted for from 30 seconds to 40 seconds prior to the measuring step. In some cases, the sample is contacted for from 20 seconds to 30 seconds prior to the measuring step. In some cases, the sample is contacted for from 10 seconds to 20 seconds prior to the measuring step.

The present disclosure provides methods of detecting a target RNA in a sample comprising a plurality of RNAs (e.g., comprising a target RNA and a plurality of non-target RNAs). In some cases, the methods comprise: a) contacting the sample with: (i) a Cas13Z guide RNA that hybridizes with the target RNA, and (ii) a Cas13Z protein that cleaves RNAs present in the sample; and b) measuring a detectable signal produced by Cas13Z protein-mediated RNA cleavage. In some cases, a method of the present disclosure provides for substantially simultaneous detection of two different target RNAs (a first target RNA and a second target RNA) in a sample.

A method of the present disclosure for detecting a target RNA (e.g., a single-stranded target RNA) in a sample comprising a plurality of RNAs (including the target RNA and a plurality of non-target RNAs) can detect a target RNA with a high degree of sensitivity. In some cases, a method of the present disclosure can be used to detect a target RNA present in a sample comprising a plurality of RNAs (including the target RNA and a plurality of non-target RNAs), where the target RNA is present at one or more copies per 10⁷ non-target RNAs (e.g., one or more copies per 10⁶ non-target RNAs, one or more copies per 10⁵ non-target RNAs, one or more copies per 10⁴ non-target RNAs, one or more copies per 10³ non-target RNAs, one or more copies per 10² non-target RNAs, one or more copies per 50 non-target RNAs, one or more copies per 20 non-target RNAs, one or more copies per 10 non-target RNAs, or one or more copies per 5 non-target RNAs).

In some cases, a method of the present disclosure can detect a target RNA present in a sample comprising a plurality of RNAs (including the target RNA and a plurality of non-target RNAs), where the target RNA is present at from one copy per 10⁷ non-target RNAs to one copy per 10 non-target RNAs (e.g., from 1 copy per 10⁷ non-target RNAs to 1 copy per 10² non-target RNAs, from 1 copy per 10⁷ non-target RNAs to 1 copy per 10³ non-target RNAs, from 1 copy per 10⁷ non-target RNAs to 1 copy per 10⁴ non-target RNAs, from 1 copy per 10⁷ non-target RNAs to 1 copy per 10⁵ non-target RNAs, from 1 copy per 10⁷ non-target RNAs to 1 copy per 10⁶ non-target RNAs, from 1 copy per 10⁶ non-target RNAs to 1 copy per 10 non-target RNAs, from 1 copy per 10⁶ non-target RNAs to 1 copy per 10² non-target RNAs, from 1 copy per 10⁶ non-target RNAs to 1 copy per 10³ non-target RNAs, from 1 copy per 10⁶ non-target RNAs to 1 copy per 10⁴ non-target RNAs, from 1 copy per 10⁶ non-target RNAs to 1 copy per 10⁵ non-target RNAs, from 1 copy per 10⁵ non-target RNAs to 1 copy per 10 non-target RNAs, from 1 copy per 10⁵ non-target RNAs to 1 copy per 10² non-target RNAs, from 1 copy per 10⁵ non-target RNAs to 1 copy per 10³ non-target RNAs, or from 1 copy per 10⁵ non-target RNAs to 1 copy per 10⁴ non-target RNAs).

In some cases, a method of the present disclosure can detect a target RNA present in a sample comprising a plurality of RNAs (including the target RNA and a plurality of non-target RNAs), where the target single-stranded RNA is present at from one copy per 10⁷ non-target RNAs to one copy per 100 non-target RNAs (e.g., from 1 copy per 10⁷ non-target RNAs to 1 copy per 10² non-target RNAs, from 1 copy per 10⁷ non-target RNAs to 1 copy per 10³ non-target RNAs, from 1 copy per 10⁷ non-target RNAs to 1 copy per 10⁴ non-target RNAs, from 1 copy per 10⁷ non-target RNAs to 1 copy per 10⁵ non-target RNAs, from 1 copy per 10⁷ non-target RNAs to 1 copy per 10⁶ non-target RNAs, from 1 copy per 10⁶ non-target RNAs to 1 copy per 100 non-target RNAs, from 1 copy per 10⁶ non-target RNAs to 1 copy per 10² non-target RNAs, from 1 copy per 10⁶ non-target RNAs to 1 copy per 10³ non-target RNAs, from 1 copy per 10⁶ non-target RNAs to 1 copy per 10⁴ non-target RNAs, from 1 copy per 10⁶ non-target RNAs to 1 copy per 10⁵ non-target RNAs, from 1 copy per 10⁵ non-target RNAs to 1 copy per 100 non-target RNAs, from 1 copy per 10⁵ non-target RNAs to 1 copy per 10² non-target RNAs, from 1 copy per 10⁵ non-target RNAs to 1 copy per 10³ non-target RNAs, or from 1 copy per 10⁵ non-target RNAs to 1 copy per 10⁴ non-target RNAs).

In some cases, the threshold of detection, for a subject method of detecting a target RNA in a sample, is 10 nM or less. The term “threshold of detection” is used herein to describe the minimal amount of target RNA that must be present in a sample in order for detection to occur. Thus, as an illustrative example, when a threshold of detection is 10 nM, then a signal can be detected when a target RNA is present in the sample at a concentration of 10 nM or more. In some cases, a method of the present disclosure has a threshold of detection of 5 nM or less. In some cases, a method of the present disclosure has a threshold of detection of 1 nM or less. In some cases, a method of the present disclosure has a threshold of detection of 0.5 nM or less. In some cases, a method of the present disclosure has a threshold of detection of 0.1 nM or less. In some cases, a method of the present disclosure has a threshold of detection of 0.05 nM or less. In some cases, a method of the present disclosure has a threshold of detection of 0.01 nM or less. In some cases, a method of the present disclosure has a threshold of detection of 0.005 nM or less. In some cases, a method of the present disclosure has a threshold of detection of 0.001 nM or less. In some cases, a method of the present disclosure has a threshold of detection of 0.0005 nM or less. In some cases, a method of the present disclosure has a threshold of detection of 0.0001 nM or less. In some cases, a method of the present disclosure has a threshold of detection of 0.00005 nM or less. In some cases, a method of the present disclosure has a threshold of detection of 0.00001 nM or less. In some cases, a method of the present disclosure has a threshold of detection of 10 pM or less. In some cases, a method of the present disclosure has a threshold of detection of 1 pM or less. In some cases, a method of the present disclosure has a threshold of detection of 500 fM or less. In some cases, a method of the present disclosure has a threshold of detection of 250 fM or less. In some cases, a method of the present disclosure has a threshold of detection of 100 fM or less. In some cases, a method of the present disclosure has a threshold of detection of 50 fM or less.

In some cases, the threshold of detection (for detecting the target RNA in a subject method), is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM)(where the concentration refers to the threshold concentration of target RNA at which the target RNA can be detected). In some cases, a method of the present disclosure has a threshold of detection in a range of from 800 fM to 100 pM. In some cases, a method of the present disclosure has a threshold of detection in a range of from 1 pM to 10 pM. In some cases, a method of the present disclosure has a threshold of detection in a range of from 10 fM to 500 fM, e.g., from 10 fM to 50 fM, from 50 fM to 100 fM, from 100 fM to 250 fM, or from 250 fM to 500 fM.

In some cases, the minimum concentration at which a target RNA can be detected in a sample is in a range of from 500 fM to 1 nM (e.g., from 500 fM to 500 pM, from 500 fM to 200 pM, from 500 fM to 100 pM, from 500 fM to 10 pM, from 500 fM to 1 pM, from 800 fM to 1 nM, from 800 fM to 500 pM, from 800 fM to 200 pM, from 800 fM to 100 pM, from 800 fM to 10 pM, from 800 fM to 1 pM, from 1 pM to 1 nM, from 1 pM to 500 pM, from 1 pM to 200 pM, from 1 pM to 100 pM, or from 1 pM to 10 pM). In some cases, the minimum concentration at which a single stranded target RNA can be detected in a sample is in a range of from 800 fM to 100 pM. In some cases, the minimum concentration at which a target RNA can be detected in a sample is in a range of from 1 pM to 10 pM.

In some cases, a method of the present disclosure can detect a target RNA present in a sample comprising a plurality of RNAs (including the target RNA and a plurality of non-target RNAs), where the target RNA is present at a concentration as low as 500 fM (e.g., as low as 800 fM, as low as 1 pM, as low as 10 pM or as low as 100 pM). In some cases, a method of the present disclosure can detect a target RNA present in a sample comprising a plurality of RNAs (including the target RNA and a plurality of non-target RNAs), where the target single-stranded RNA is present at a concentration as low as 1 pM.

In some cases, a method of the present disclosure can detect a target RNA present in a sample comprising a plurality of RNAs (including the target RNA and a plurality of non-target RNAs), where the target single-stranded RNA is present at a concentration as low as 500 fM (e.g., as low as 800 fM, as low as 1 pM, as low as 10 pM or as low as 100 pM), and where the sample is contacted for 60 minutes or less prior to the measuring step (e.g., in some cases 40 minutes or less). In some cases, a method of the present disclosure can detect a target RNA present in a sample comprising a plurality of RNAs (including the target RNA and a plurality of non-target RNAs), where the target RNA is present at a concentration as low as 1 pM, and where the sample is contacted for 60 minutes or less prior to the measuring step (e.g., in some cases 40 minutes or less).

For example, in some cases, a method of the present disclosure provides for detection of a target RNA present in a sample at a concentration of 500 fM or more (e.g., 800 fM or more, 1 pM or more, 5 pM or more, 10 pM or more). In some cases, a method of the present disclosure provides for detection of a target RNA present in a sample at a concentration of 1 pM or more (e.g., 2 pM or more 5 pM or more, or 8 pM or more). In some cases, a method of the present disclosure provides for detection of a target RNA present in a sample at a concentration of 500 fM or more (e.g., 1 pM or more, 5 pM or more, 10 pM or more), where the sample is contacted for 60 minutes or less prior to the measuring step (e.g., in some cases 40 minutes or less). In some cases, a method of the present disclosure provides for detection of a target RNA present in a sample at a concentration of 1 pM or more (e.g., 2 pM or more 5 pM or more, or 8 pM or more) where the sample is contacted for 60 minutes or less prior to the measuring step (e.g., in some cases 40 minutes or less).

In some cases, a method of the present disclosure provides for detection of a target RNA present in a sample at a concentration of 10 nM or less. In some cases, a method of the present disclosure provides for detection of a target RNA present in a sample at a concentration of 5 nM or less. In some cases, a method of the present disclosure provides for detection of a target RNA present in a sample at a concentration of 1 nM or less. In some cases, a method of the present disclosure provides for detection of a target RNA present in a sample at a concentration of 0.5 nM or less. In some cases, a method of the present disclosure provides for detection of a target RNA present in a sample at a concentration of 0.1 nM or less. In some cases, a method of the present disclosure provides for detection of a target RNA present in a sample at a concentration of 0.05 nM or less. In some cases, a method of the present disclosure provides for detection of a target RNA present in a sample at a concentration of 0.01 nM or less. In some cases, a method of the present disclosure provides for detection of a target RNA present in a sample at a concentration of 0.005 nM or less. In some cases, a method of the present disclosure provides for detection of a target RNA present in a sample at a concentration of 0.001 nM or less. In some cases, a method of the present disclosure provides for detection of a target RNA present in a sample at a concentration of 0.0005 nM or less. In some cases, a method of the present disclosure provides for detection of a target RNA present in a sample at a concentration of 0.0001 nM or less. In some cases, a method of the present disclosure provides for detection of a target RNA present in a sample at a concentration of 0.00005 nM or less. In some cases, a method of the present disclosure provides for detection of a target RNA present in a sample at a concentration of 0.00001 nM or less.

In some cases, a method of the present disclosure provides for detection of a target RNA present in a sample at a concentration of from 10⁶ nM to 1 nM, e.g., from 10-6 nM to 5×10⁻⁶ nM, from 5×106 nM to 10⁵ nM, from 105 nM to 5×10⁵ nM, from 5×10⁵ nM to 10-4 nM, from 104 nM to 5×10⁻⁴ nM, from 5×10⁴ nM to 10-3 nM, from 10-3 nM to 5×10³ nM, from 5×10⁻³ nM to 10-2 nM, from 10-2 nM to 5×10⁻² nM, from 5×10⁻² nM to 0.1 nM, from 0.1 nM to 0.5 nM, from 0.5 nM to 1 nM, from 1 nM to 5 nM, or from 5 nM to 10 nM.

In some cases, a method of the present disclosure provides for detection of a target RNA present in a sample at a concentration of less than 10 nM. In some cases, a method of the present disclosure provides for detection of a target RNA present in a sample at a concentration of less than 5 nM. In some cases, a method of the present disclosure provides for detection of a target RNA present in a sample at a concentration of less than 1 nM. In some cases, a method of the present disclosure provides for detection of a target RNA present in a sample at a concentration of less than 0.5 nM. In some cases, a method of the present disclosure provides for detection of a target RNA present in a sample at a concentration of less than 0.1 nM. In some cases, a method of the present disclosure provides for detection of a target RNA present in a sample at a concentration of less than 0.05 nM. In some cases, a method of the present disclosure provides for detection of a target RNA present in a sample at a concentration of less than 0.01 nM. In some cases, a method of the present disclosure provides for detection of a target RNA present in a sample at a concentration of less than 0.005 nM. In some cases, a method of the present disclosure provides for detection of a target RNA present in a sample at a concentration of less than 0.001 nM. In some cases, a method of the present disclosure provides for detection of a target RNA present in a sample at a concentration of less than 0.0005 nM. In some cases, a method of the present disclosure provides for detection of a target RNA present in a sample at a concentration of less than 0.0001 nM. In some cases, a method of the present disclosure provides for detection of a target RNA present in a sample at a concentration of less than 0.00005 nM. In some cases, a method of the present disclosure provides for detection of a target RNA present in a sample at a concentration of less than 0.00001 nM.

In some cases, a method of the present disclosure can be used to determine the amount of a target RNA in a sample (e.g., a sample comprising the target RNA and a plurality of non-target RNAs). Determining the amount of a target RNA in a sample can comprise comparing the amount of detectable signal generated from a test sample to the amount of detectable signal generated from a reference sample. Determining the amount of a target RNA in a sample can comprise: measuring the detectable signal to generate a test measurement; measuring a detectable signal produced by a reference sample to generate a reference measurement; and comparing the test measurement to the reference measurement to determine an amount of target RNA present in the sample.

For example, in some cases, a method of the present disclosure for determining the amount of a target RNA in a sample comprises: a) contacting the sample (e.g., a sample comprising the target RNA and a plurality of non-target RNAs) with: (i) a Cas13Z guide RNA that hybridizes with the single stranded target RNA, and (ii) a Cas13Z protein that cleaves RNAs present in the sample; b) measuring a detectable signal produced by Cas13Z protein-mediated RNA cleavage, generating a test measurement; c) measuring a detectable signal produced by a reference sample to generate a reference measurement; and d) comparing the test measurement to the reference measurement to determine an amount of target RNA present in the sample.

As another example, in some cases, a method of the present disclosure for determining the amount of a target RNA in a sample comprises: a) contacting the sample (e.g., a sample comprising the target RNA and a plurality of non-target RNAs) with: i) a precursor Cas13Z guide RNA array comprising two or more Cas 13Z guide RNAs each of which has a different guide sequence; and (ii) a Cas13Z protein that cleaves the precursor Cas13Z guide RNA array into individual Cas13Z guide RNAs, and also cleaves RNAs of the sample; b) measuring a detectable signal produced by Cas13Z protein-mediated RNA cleavage, generating a test measurement; c) measuring a detectable signal produced by each of two or more reference samples to generate two or more reference measurements; and d) comparing the test measurement to the reference measurements to determine an amount of target RNA present in the sample.

### Samples

A subject sample includes a plurality of target RNAs. The term “plurality” is used herein to mean two or more. Thus, in some cases a sample includes two or more (e.g., 3 or more, 5 or more, 10 or more, 20 or more, 50 or more, 100 or more, 500 or more, 1,000 or more, or 5,000 or more) RNAs. A subject method can be used as a very sensitive way to detect a single stranded target RNA present in a complex mixture of RNAs. Thus, in some cases the sample includes 5 or more RNAs (e.g., 10 or more, 20 or more, 50 or more, 100 or more, 500 or more, 1,000 or more, or 5,000 or more RNAs) that differ from one another in sequence. In some cases, the sample includes 10 or more, 20 or more, 50 or more, 100 or more, 500 or more, 10³ or more, 5×10³ or more, 10⁴ or more, 5×10⁴ or more, 10⁵ or more, 5×10⁵ or more, 10⁶ or more 5×10⁶ or more, or 10⁷ or more, RNAs that differ from one another in sequence. In some cases, the sample comprises from 10 to 20, from 20 to 50, from 50 to 100, from 100 to 500, from 500 to 10³, from 10³ to 5×10³, from 5×10³ to 10⁴, from 10⁴ to 5×10⁴, from 5×10⁴ to 10⁵, from 10⁵ to 5×10⁵, from 5×10⁵ to 10⁶, from 10⁶ to 5×10⁶, or from 5×106 to 10⁷, or more than 10⁷, RNAs that differ from one another in sequence. In some cases, the sample comprises from 5 to 10⁷ RNAs that differ from one another in sequence (e.g., from 5 to 10⁶, from 5 to 10⁵, from 5 to 50,000, from 5 to 30,000, from 10 to 10⁶, from 10 to 10⁵, from 10 to 50,000, from 10 to 30,000, from 20 to 10⁶, from 20 to 105, from 20 to 50,000, or from 20 to 30,000 RNAs that differ from one another in sequence). In some cases, the sample comprises from 5 to 50,000 RNAs that differ from one another in sequence (e.g., from 5 to 30,000, from 10 to 50,000, or from 10 to 30,000) RNAs that differ from one another in sequence). In some cases the sample includes 20 or more RNAs that differ from one another in sequence. In some cases, the sample includes RNAs from a cell lysate (e.g., a eukaryotic cell lysate, a mammalian cell lysate, a human cell lysate, a prokaryotic cell lysate, a plant cell lysate, and the like). For example, in some cases the sample includes expressed RNAs from a cell such as a eukaryotic cell, e.g., a mammalian cell such as a human cell.

The term “sample” is used herein to mean any sample that includes single stranded RNA. The sample can be derived from any source, e.g., the sample can be a synthetic combination of purified RNAs; the sample can be a cell lysate, an RNA-enriched cell lysate, or RNAs isolated and/or purified from a cell lysate. The sample can be from a patient (e.g., for the purpose of diagnosis). The sample can be from permeabilized cells. The sample can be from crosslinked cells. The sample can be in tissue sections. The sample can be from tissues prepared by crosslinking followed by delipidation and adjustment to make a uniform refractive index. Examples of tissue preparation by crosslinking followed by delipidation and adjustment to make a uniform refractive index have been described in, for example, Shah et al., Development (2016) 143, 2862-2867 doi: 10.1242/dev.138560.

A “sample” can include a target RNA and a plurality of non-target RNAs. In some cases, the target RNA is present in the sample at one copy per 10 non-target RNAs, one copy per 20 non-target RNAs, one copy per 25 non-target RNAs, one copy per 50 non-target RNAs, one copy per 100 non-target RNAs, one copy per 500 non-target RNAs, one copy per 10³ non-target RNAs, one copy per 5×103 non-target RNAs, one copy per 10⁴ non-target RNAs, one copy per 5×10⁴ non-target RNAs, one copy per 10⁵ non-target RNAs, one copy per 5×10⁵ non-target RNAs, one copy per 10⁶ non-target RNAs, or less than one copy per 10⁶ non-target RNAs. In some cases, the target single-stranded RNA is present in the sample at from one copy per 10 non-target RNAs to 1 copy per 20 non-target RNAs, from 1 copy per 20 non-target RNAs to 1 copy per 50 non-target RNAs, from 1 copy per 50 non-target RNAs to 1 copy per 100 non-target RNAs, from 1 copy per 100 non-target RNAs to 1 copy per 500 non-target RNAs, from 1 copy per 500 non-target RNAs to 1 copy per 10³ non-target RNAs, from 1 copy per 10³ non-target RNAs to 1 copy per 5×10³ non-target RNAs, from 1 copy per 5×10³ non-target RNAs to 1 copy per 10⁴ non-target RNAs, from 1 copy per 10⁴ non-target RNAs to 1 copy per 10⁵ non-target RNAs, from 1 copy per 10⁵ non-target RNAs to 1 copy per 10⁶ non-target RNAs, or from 1 copy per 10⁶ non-target RNAs to 1 copy per 10⁷ non-target RNAs.

Suitable samples include but are not limited to blood, serum, plasma, urine, aspirate, and biopsy samples. Thus, the term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., RNAs. The term “sample” encompasses biological samples such as a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A “biological sample” includes biological fluids derived therefrom (e.g., cancerous cell, infected cell, etc.), e.g., a sample comprising RNAs that is obtained from such cells (e.g., a cell lysate or other cell extract comprising RNAs).

A sample can comprise, or can be obtained from, any of a variety of cells, tissues, organs, or acellular fluids. Suitable sample sources include eukaryotic cells, bacterial cells, and archaeal cells. Suitable sample sources include single-celled organisms and multi-cellular organisms. Suitable sample sources include single-cell eukaryotic organisms; a plant or a plant cell; an algal cell, e.g., Botryococcus braunii, Chlamydomonas reinhardtii, Nannochloropsis gaditana, Chlorella pyrenoidosa, Sargassum patens, C. agardh, and the like; a fungal cell (e.g., a yeast cell); an animal cell, tissue, or organ; a cell, tissue, or organ from an invertebrate animal (e.g. fruit fly, cnidarian, echinoderm, nematode, an insect, an arachnid, etc.); a cell, tissue, fluid, or organ from a vertebrate animal (e.g., fish, amphibian, reptile, bird, mammal); a cell, tissue, fluid, or organ from a mammal (e.g., a human; a non-human primate; an ungulate; a feline; a bovine; an ovine; a caprine; etc.). Suitable sample sources include nematodes, protozoans, and the like. Suitable sample sources include parasites such as helminths, malarial parasites, etc.

Suitable sample sources include a cell, tissue, or organism of any of the six kingdoms, e.g., Bacteria (e.g., Eubacteria); Archaebacteria; Protista; Fungi; Plantae; and Animalia. Suitable sample sources include plant-like members of the kingdom Protista, including, but not limited to, algae (e.g., green algae, red algae, glaucophytes, cyanobacteria); fungus-like members of Protista, e.g., slime molds, water molds, etc.; animal-like members of Protista, e.g., flagellates (e.g., Euglena), amoeboids (e.g., amoeba), sporozoans (e.g, Apicomplexa, Myxozoa, Microsporidia), and ciliates (e.g., Paramecium). Suitable sample sources include include members of the kingdom Fungi, including, but not limited to, members of any of the phyla: Basidiomycota (club fungi; e.g., members of Agaricus, Amanita, Boletus, Cantherellus, etc.); Ascomycota (sac fungi, including, e.g., Saccharomyces); Mycophycophyta (lichens); Zygomycota (conjugation fungi); and Deuteromycota. Suitable sample sources include include members of the kingdom Plantae, including, but not limited to, members of any of the following divisions: Bryophyta (e.g., mosses), Anthocerotophyta (e.g., hornworts), Hepaticophyta (e.g., liverworts), Lycophyta (e.g., club mosses), Sphenophyta (e.g., horsetails), Psilophyta (e.g., whisk ferns), Ophioglossophyta, Pterophyta (e.g., ferns), Cycadophyta, Gingkophyta, Pinophyta, Gnetophyta, and Magnoliophyta (e.g., flowering plants). Suitable sample sources include include members of the kingdom Animalia, including, but not limited to, members of any of the following phyla: Porifera (sponges); Placozoa; Orthonectida (parasites of marine invertebrates); Rhombozoa; Cnidaria (corals, anemones, jellyfish, sea pens, sea pansies, sea wasps); Ctenophora (comb jellies); Platyhelminthes (flatworms); Nemertina (ribbon worms); Ngathostomulida (jawed worms) p Gastrotricha; Rotifera; Priapulida; Kinorhyncha; Loricifera; Acanthocephala; Entoprocta; Nemotoda; Nematomorpha; Cycliophora; Mollusca (mollusks); Sipuncula (peanut worms); Annelida (segmented worms); Tardigrada (water bears); Onychophora (velvet worms); Arthropoda (including the subphyla: Chelicerata, Myriapoda, Hexapoda, and Crustacea, where the Chelicerata include, e.g., arachnids, Merostomata, and Pycnogonida, where the Myriapoda include, e.g., Chilopoda (centipedes), Diplopoda (millipedes), Paropoda, and Symphyla, where the Hexapoda include insects, and where the Crustacea include shrimp, krill, barnacles, etc.; Phoronida; Ectoprocta (moss animals); Brachiopoda; Echinodermata (e.g. starfish, sea daisies, feather stars, sea urchins, sea cucumbers, brittle stars, brittle baskets, etc.); Chaetognatha (arrow worms); Hemichordata (acorn worms); and Chordata. Suitable members of Chordata include any member of the following subphyla: Urochordata (sea squirts; including Ascidiacea, Thaliacea, and Larvacea); Cephalochordata (lancelets); Myxini (hagfish); and Vertebrata, where members of Vertebrata include, e.g., members of Petromyzontida (lampreys), Chondrichthyces (cartilaginous fish), Actinopterygii (ray-finned fish), Actinista (coelocanths), Dipnoi (lungfish), Reptilia (reptiles, e.g., snakes, alligators, crocodiles, lizards, etc.), Aves (birds); and Mammalian (mammals). Suitable plants include any monocotyledon and any dicotyledon.

Suitable sources of a sample include cells, fluid, tissue, or organ taken from an organism; from a particular cell or group of cells isolated from an organism; etc. For example, where the organism is a plant, suitable sources include xylem, the phloem, the cambium layer, leaves, roots, etc. Where the organism is an animal, suitable sources include particular tissues (e.g., lung, liver, heart, kidney, brain, spleen, skin, fetal tissue, etc.), or a particular cell type (e.g., neuronal cells, epithelial cells, endothelial cells, astrocytes, macrophages, glial cells, islet cells, T lymphocytes, B lymphocytes, etc.).

In some cases, the source of the sample is a diseased cell, fluid, tissue, or organ. In some cases, the source of the sample is a normal (non-diseased) cell, fluid, tissue, or organ. In some cases, the source of the sample is a pathogen-infected cell, tissue, or organ. Pathogens include viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

In some cases, the sample comprises cancer cells. In some cases, a Cas13Z guide RNA is designed to detect a cancer-specific mutation in the genome of a cancer cell. In some cases, the cancer-specific mutation confers drug resistance (e.g., resistance to a cancer chemotherapeutic drug). A cancer-specific mutation can be present in one or more genes encoding a protein selected from the group consisting of Programmed Death-Ligand 1 (PD-L1), androgen receptor (AR), Bruton's Tyrosine Kinase (BTK), Epidermal Growth Factor Receptor (EGFR), BCR-Abl, c-kit, PIK3CA, HER2, EML4-ALK, KRAS, ALK, ROS1, AKT1, BRAF, MEK1, MEK2, NRAS, RAC1, and ESR1. The cancer specific mutation may be a mutation in a gene selected from the group consisting of CASP8, B2M, PIK3CA, SMC1A, ARID5B, TET2, ALPK2, COL5A1, TP53, DNER, NCOR1, MORC4, CIC, IRF6, MYOCD, ANKLE1, CNKSR1, NF1, SOS1, ARID2, CUL4B, DDX3X, FUBP1, TCP11L2, HLA-A, B or C, CSNK2A1, MET, ASXL1, PD-L1, PD-L2, IDO1, IDO2, ALOX12B and ALOX15B. For example, a subject method can be used to detect an RNA transcript of a gene encoding a protein comprising a cancer-specific mutation.

### Target RNA

A target RNA can be any RNA (e.g., single-stranded RNA or double-stranded RNA). Examples include but are not limited to mRNA, rRNA, tRNA, non-coding RNA (ncRNA), long non-coding RNA (lncRNA), and microRNA (miRNA). In some cases, the target RNA is mRNA. In some cases, the single stranded target nucleic acid is RNA from a virus (e.g., Zika virus, human immunodeficiency virus, influenza virus, and the like). In some cases, the single-stranded target nucleic acid is RNA of a parasite. In some cases, the single-stranded target nucleic acid is RNA of a bacterium, e.g., a pathogenic bacterium. The source of the target RNA can be the same as the source of the RNA sample, as described above. In some cases, detection of a target RNA, where the target RNA is an mRNA, provides for detection of a DNA encoding the mRNA. In some cases, the target RNA is an mRNA present in a diseased cell (e.g., a cancer cell).

In some cases, a target RNA or a DNA encoding a target RNA is not subjected to an amplification step. In some cases, a target RNA or a DNA encoding a target RNA is subject to an amplification step, to generate an amplification product (an amplicon), and the amplification product is detected using a method of the present disclosure. If an amplification step is included, in some cases, the amplifying comprises recombinase polymerase amplification (RPA), transcription mediated amplification (TMA), strand displacement amplification (SDA), helicase dependent amplification (HDA), loop mediated amplification (LAMP), rolling circle amplification (RCA), single primer isothermal amplification (SPIA), ligase chain reaction (LCR), simple method amplifying RNA targets (SMART), or improved multiple displacement amplification (IMDA), or nucleic acid sequence-based amplification (NASBA). In some cases, the amplifying comprises recombinase polymerase amplification (RPA). In some cases, the amplifying comprises loop mediated amplification (LAMP).

### Measuring a Detectable Signal

In some cases, a subject method includes a step of measuring (e.g., measuring a detectable signal produced by Cas13Z protein-mediated RNA cleavage). Because a Cas13Z protein cleaves non-targeted RNA once activated, which occurs when a Cas13Z guide RNA hybridizes with a target RNA in the presence of a Cas13Z protein, a detectable signal can be any signal that is produced when RNA is cleaved. For example, in some cases the step of measuring can include one or more of: gold nanoparticle-based detection (e.g., see Xu et al., Angew Chem Int Ed Engl. 2007; 46 (19): 3468-70; and Xia et. al., Proc Natl Acad Sci USA. 2010 Jun. 15;107 (24): 10837-41), fluorescence polarization, colloid phase transition/dispersion (e.g., Baksh et. al., Nature. 2004 Jan. 8;427 (6970): 139-41), electrochemical detection, semiconductor-based sensing (e.g., Rothberg et. al., Nature. 2011 Jul. 20;475 (7356): 348-52; e.g., one could use a phosphatase to generate a pH change after RNA cleavage reactions, by opening 2′-3′ cyclic phosphates, and by releasing inorganic phosphate into solution), and detection of a labeled detector RNA (see below for more details). The readout of such detection methods can be any convenient readout. Examples of possible readouts include but are not limited to: a measured amount of detectable fluorescent signal; a visual analysis of bands on a gel (e.g., bands that represent cleaved product versus uncleaved substrate), a visual or sensor based detection of the presence or absence of a color (i.e., color detection method), and the presence or absence of (or a particular amount of) an electrical signal.

The measuring can in some cases be quantitative, e.g., in the sense that the amount of signal detected can be used to determine the amount of target RNA present in the sample. The measuring can in some cases be qualitative, e.g., in the sense that the presence or absence of detectable signal can indicate the presence or absence of targeted RNA. In some cases, a detectable signal will not be present (e.g., above a given threshold level) unless the targeted RNA(s) is present above a particular threshold concentration. In some cases, the threshold of detection can be titrated by modifying the amount of Cas13Z protein, guide RNA, sample volume, and/or detector RNA (if one is used). As such, for example, as would be understood by one of ordinary skill in the art, a number of controls can be used if desired in order to set up one or more reactions, each set up to detect a different threshold level of target RNA, and thus such a series of reactions could be used to determine the amount of target RNA present in a sample (e.g., one could use such a series of reactions to determine that a target RNA is present in the sample ‘at a concentration of at least X’).

### Labeled Detector RNA

In some cases, a subject method includes contacting a sample (e.g., a sample comprising a target RNA and a plurality of non-target RNAs) with: i) a labeled detector RNA; ii) a Cas13Z protein; and iii) a Cas13Z guide RNA (or precursor Cas13Z guide RNA array). For example, in some cases, a subject method includes contacting a sample with a labeled detector RNA comprising a fluorescence-emitting dye pair; the Cas13Z protein cleaves the labeled detector RNA after it is activated (by binding to the Cas13Z guide RNA in the context of the guide RNA hybridizing to a target RNA); and the detectable signal that is measured is produced by the fluorescence-emitting dye pair. For example, in some cases, a subject method includes contacting a sample with a labeled detector RNA comprising a fluorescence resonance energy transfer (FRET) pair or a quencher/fluor pair, or both. In some cases, a subject method includes contacting a sample with a labeled detector RNA comprising a FRET pair. In some cases, a subject method includes contacting a sample with a labeled detector RNA comprising a fluor/quencher pair. Fluorescence-emitting dye pairs comprise a FRET pair or a quencher/fluor pair. In both cases of a FRET pair and a quencher/fluor pair, the emission spectrum of one of the dyes overlaps a region of the absorption spectrum of the other dye in the pair. As used herein, the term “fluorescence-emitting dye pair” is a generic term used to encompass both a “fluorescence resonance energy transfer (FRET) pair” and a “quencher/fluor pair,” both of which terms are discussed in more detail below. The term “fluorescence-emitting dye pair” is used interchangeably with the phrase “a FRET pair and/or a quencher/fluor pair.”

In some cases (e.g., when the detector RNA includes a FRET pair) the labeled detector RNA produces an amount of detectable signal prior to being cleaved, and the amount of detectable signal that is measured is reduced when the labeled detector RNA is cleaved. In some cases, the labeled detector RNA produces a first detectable signal prior to being cleaved (e.g., from a FRET pair) and a second detectable signal when the labeled detector RNA is cleaved (e.g., from a quencher/fluor pair). As such, in some cases, the labeled detector RNA comprises a FRET pair and a quencher/fluor pair.

In some cases, the labeled detector RNA comprises a FRET pair. FRET is a process by which radiationless transfer of energy occurs from an excited state fluorophore to a second chromophore in close proximity. The range over which the energy transfer can take place is limited to approximately 10 nanometers (100 angstroms), and the efficiency of transfer is extremely sensitive to the separation distance between fluorophores. Thus, as used herein, the term “FRET” (“fluorescence resonance energy transfer”; also known as “Förster resonance energy transfer”) refers to a physical phenomenon involving a donor fluorophore and a matching acceptor fluorophore selected so that the emission spectrum of the donor overlaps the excitation spectrum of the acceptor, and further selected so that when donor and acceptor are in close proximity (usually 10 nm or less) to one another, excitation of the donor will cause excitation of and emission from the acceptor, as some of the energy passes from donor to acceptor via a quantum coupling effect. Thus, a FRET signal serves as a proximity gauge of the donor and acceptor; only when they are in close proximity to one another is a signal generated. The FRET donor moiety (e.g., donor fluorophore) and FRET acceptor moiety (e.g., acceptor fluorophore) are collectively referred to herein as a “FRET pair”.

The donor-acceptor pair (a FRET donor moiety and a FRET acceptor moiety) is referred to herein as a “FRET pair” or a “signal FRET pair.” Thus, in some cases, a subject labeled detector RNA includes two signal partners (a signal pair), when one signal partner is a FRET donor moiety and the other signal partner is a FRET acceptor moiety. A subject labeled detector RNA that includes such a FRET pair (a FRET donor moiety and a FRET acceptor moiety) will thus exhibit a detectable signal (a FRET signal) when the signal partners are in close proximity (e.g., while on the same RNA molecule), but the signal will be reduced (or absent) when the partners are separated (e.g., after cleavage of the RNA molecule by a Cas13Z protein).

FRET donor and acceptor moieties (FRET pairs) will be known to one of ordinary skill in the art and any convenient FRET pair (e.g., any convenient donor and acceptor moiety pair) can be used. Examples of suitable FRET pairs include but are not limited to those presented in Table 1. See also: Bajar et al. Sensors (Basel). 2016 Sep. 14;16 (9); and Abraham et al. PLOS One. 2015 Aug. 3;10 (8): e0134436.

TABLE 1
Examples of FRET pairs (donor and acceptor FRET moieties)

| Donor | Acceptor |
|---|---|
| Tryptophan | Dansyl |
| IAEDANS (1) | DDPM (2) |
| BFP | DsRFP |
| Dansyl | Fluorescein |
| | isothiocyanate (FITC) |
| Dansyl | Octadecylrhodamine |
| Cyan fluorescent | Green fluorescent protein |
| protein (CFP) | (GFP) |
| CF (3) | Texas Red |
| Fluorescein | Tetramethylrhodamine |
| Cy3 | Cy5 |
| GFP | Yellow fluorescent |
| | protein (YFP) |
| BODIPY FL (4) | BODIPY FL (4) |
| Rhodamine 110 | Cy3 |
| Rhodamine 6G | Malachite Green |
| FITC | Eosin Thiosemicarbazide |
| B-Phycoerythrin | Cy5 |
| Cy5 | Cy5.5 |

(1) 5-(2-iodoacetylaminoethyl)aminonaphthalene-1-sulfonic acid
(2) N-(4-dimethylamino-3,5-dinitrophenyl)maleimide
(3) carboxyfluorescein succinimidyl ester
(4) 4,4-difluoro-4-bora-3a,4a-diaza-s-indacene

In some cases, a detectable signal is produced when the labeled detector RNA is cleaved (e.g., in some cases, the labeled detector RNA comprises a quencher/fluor pair. One signal partner of a signal quenching pair produces a detectable signal and the other signal partner is a quencher moiety that quenches the detectable signal of the first signal partner (i.e., the quencher moiety quenches the signal of the signal moiety such that the signal from the signal moiety is reduced (quenched) when the signal partners are in proximity to one another, e.g., when the signal partners of the signal pair are in close proximity).

For example, in some cases, an amount of detectable signal increases when the labeled detector RNA is cleaved. For example, in some cases, the signal exhibited by one signal partner (a signal moiety) is quenched by the other signal partner (a quencher signal moiety), e.g., when both are present on the same RNA molecule prior to cleavage by a Cas 13Z protein. Such a signal pair is referred to herein as a “quencher/fluor pair”, “quenching pair”, or “signal quenching pair.” For example, in some cases, one signal partner (e.g., the first signal partner) is a signal moiety that produces a detectable signal that is quenched by the second signal partner (e.g., a quencher moiety). The signal partners of such a quencher/fluor pair will thus produce a detectable signal when the partners are separated (e.g., after cleavage of the detector RNA by a Cas13Z protein), but the signal will be quenched when the partners are in close proximity (e.g., prior to cleavage of the detector RNA by a Cas13Z protein).

A quencher moiety can quench a signal from the signal moiety (e.g., prior to cleave of the detector RNA by a Cas13Z protein) to various degrees. In some cases, a quencher moiety quenches the signal from the signal moiety where the signal detected in the presence of the quencher moiety (when the signal partners are in proximity to one another) is 95% or less of the signal detected in the absence of the quencher moiety (when the signal partners are separated). For example, in some cases, the signal detected in the presence of the quencher moiety can be 90% or less, 80% or less, 70% or less, 60% or less, 50% or less, 40% or less, 30% or less, 20% or less, 15% or less, 10% or less, or 5% or less of the signal detected in the absence of the quencher moiety. In some cases, no signal (e.g., above background) is detected in the presence of the quencher moiety.

In some cases, the signal detected in the absence of the quencher moiety (when the signal partners are separated) is at least 1.2 fold greater (e.g., at least 1.3fold, at least 1.5 fold, at least 1.7 fold, at least 2 fold, at least 2.5 fold, at least 3 fold, at least 3.5 fold, at least 4 fold, at least 5 fold, at least 7 fold, at least 10 fold, at least 20 fold, or at least 50 fold greater) than the signal detected in the presence of the quencher moiety (when the signal partners are in proximity to one another).

In some cases, the signal moiety is a fluorescent label. In some such cases, the quencher moiety quenches the signal (the light signal) from the fluorescent label (e.g., by absorbing energy in the emission spectra of the label). Thus, when the quencher moiety is not in proximity with the signal moiety, the emission (the signal) from the fluorescent label is detectable because the signal is not absorbed by the quencher moiety. Any convenient donor acceptor pair (signal moiety/quencher moiety pair) can be used and many suitable pairs are known in the art.

In some cases, the quencher moiety absorbs energy from the signal moiety (also referred to herein as a “detectable label”) and then emits a signal (e.g., light at a different wavelength). Thus, in some cases, the quencher moiety is itself a signal moiety (e.g., a signal moiety can be 6-carboxyfluorescein while the quencher moiety can be 6-carboxy-tetramethylrhodamine), and in some such cases, the pair could also be a FRET pair. In some cases, a quencher moiety is a dark quencher. A dark quencher can absorb excitation energy and dissipate the energy in a different way (e.g., as heat). Thus, a dark quencher has minimal to no fluorescence of its own (does not emit fluorescence). Examples of dark quenchers are further described in U.S. Pat. Nos. 8,822,673 and 8,586,718; U.S. patent publications 20140378330, 20140349295, and 20140194611; and international patent applications: WO200142505 and WO200186001, all if which are hereby incorporated by reference in their entirety.

Examples of fluorescent labels include, but are not limited to: an Alexa Fluor® dye, an ATTO dyc (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein isothiocyanate (FITC), tetramethylrhodamine (TRITC), Texas Red, Oregon Green, Pacific Blue, Pacific Green, Pacific Orange, quantum dots, and a tethered fluorescent protein.

In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Bluc, Pacific Green, and Pacific Orangc.

In some cases, a detectable label is a fluorescent label selected from: an Alexa Fluor® dye, an ATTO dye (e.g., ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, ATTO 740), a DyLight dye, a cyanine dye (e.g., Cy2, Cy3, Cy3.5, Cy3b, Cy5, Cy5.5, Cy7, Cy7.5), a FluoProbes dye, a Sulfo Cy dye, a Seta dye, an IRIS Dye, a SeTau dye, an SRfluor dye, a Square dye, fluorescein (FITC), tetramethylrhodamine (TRITC), Texas Red, Oregon Green, Pacific Blue, Pacific Green, Pacific Orange, a quantum dot, and a tethered fluorescent protein.

Examples of ATTO dyes include, but are not limited to: ATTO 390, ATTO 425, ATTO 465, ATTO 488, ATTO 495, ATTO 514, ATTO 520, ATTO 532, ATTO Rho6G, ATTO 542, ATTO 550, ATTO 565, ATTO Rho3B, ATTO Rho11, ATTO Rho12, ATTO Thio12, ATTO Rho101, ATTO 590, ATTO 594, ATTO Rho13, ATTO 610, ATTO 620, ATTO Rho14, ATTO 633, ATTO 647, ATTO 647N, ATTO 655, ATTO Oxa12, ATTO 665, ATTO 680, ATTO 700, ATTO 725, and ATTO 740.

Examples of AlexaFluor dyes include, but are not limited to: Alexa Fluor® 350, Alcxa Fluor® 405, Alexa Fluor® 430, Alexa Fluor® 488, Alexa Fluor® 500, Alexa Fluor® 514, Alcxa Fluor® 532, Alexa Fluor® 546, Alexa Fluor® 555, Alexa Fluor® 568, Alexa Fluor® 594, Alexa Fluor® 610, Alexa Fluor® 633, Alexa Fluor® 635, Alexa Fluor® 647, Alexa Fluor® 660, Alexa Fluor® 680, Alexa Fluor® 700, Alexa Fluor® 750, Alexa Fluor® 790, and the like.

Examples of quencher moieties include, but are not limited to: a dark quencher, a Black Hole Quencher® (BHQ®)(e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and metal clusters such as gold nanoparticles, and the like.

In some cases, a quencher moiety is selected from: a dark quencher, a Black Hole Quencher® (BHQ®)(e.g., BHQ-0, BHQ-1, BHQ-2, BHQ-3), a Qxl quencher, an ATTO quencher (e.g., ATTO 540Q, ATTO 580Q, and ATTO 612Q), dimethylaminoazobenzenesulfonic acid (Dabsyl), Iowa Black RQ, Iowa Black FQ, IRDye QC-1, a QSY dye (e.g., QSY 7, QSY 9, QSY 21), AbsoluteQuencher, Eclipse, and a metal cluster.

Examples of an ATTO quencher include, but are not limited to: ATTO 540Q, ATTO 580Q, and ATTO 612Q. Examples of a Black Hole Quencher® (BHQ®) include, but are not limited to: BHQ-0 (493 nm), BHQ-1 (534 nm), BHQ-2 (579 nm) and BHQ-3 (672 nm).

For examples of some detectable labels (e.g., fluorescent dyes) and/or quencher moieties, see, e.g., Bao et al., Annu Rev Biomed Eng. 2009; 11:25-47; as well as U.S. Pat. Nos. 8,822,673 and 8,586,718; U.S. patent publications 20140378330, 20140349295, 20140194611, 20130323851, 20130224871, 20110223677, 20110190486, 20110172420, 20060179585 and 20030003486; and international patent applications: WO200142505 and WO200186001, all of which are hereby incorporated by reference in their entirety.

In some cases, cleavage of a labeled detector RNA can be detected by measuring a colorimetric read-out. For example, the liberation of a fluorophore (e.g., liberation from a FRET pair, liberation from a quencher/fluor pair, and the like) can result in a wavelength shift (and thus color shift) of a detectable signal. Thus, in some cases, cleavage of a subject labeled detector RNA can be detected by a color-shift. Such a shift can be expressed as a loss of an amount of signal of one color (wavelength), a gain in the amount of another color, a change in the ration of one color to another, and the like.

### Nucleic Acid Modifications

In some cases, a labeled detector RNA comprises one or more modifications, e.g., a base modification, a backbone modification, a sugar modification, etc., to provide the nucleic acid with a new or enhanced feature (e.g., improved stability). As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally suitable. In addition, linear compounds may have internal nucleotide base complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

### Modified Backbones and Modified Internucleoside Linkages

Examples of suitable modifications include modified nucleic acid backbones and non-natural internucleoside linkages. Nucleic acids having modified backbones include those that retain a phosphorus atom in the backbone and those that do not have a phosphorus atom in the backbone.

Suitable modified oligonucleotide backbones containing a phosphorus atom therein include, for example, phosphorothioates, chiral phosphorothioates, phosphorodithioates, phosphotricsters, aminoalkylphosphotricsters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, phosphorodiamidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be a basic (the nucleobase is missing or has a hydroxyl group in place thereof). Various salts (such as, for example, potassium or sodium), mixed salts and free acid forms are also included.

In some cases, a labeled detector RNA comprises one or more phosphorothioate and/or heteroatom internucleoside linkages, in particular —CH₂—NH—O—CH₂—, —CH₂—N(CH₃)—O—CH₂— (known as a methylene(methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). MMI type internucleoside linkages are disclosed in the above referenced U.S. Pat. No. 5,489,677. Suitable amide internucleoside linkages are disclosed in t U.S. Pat. No. 5,602,240.

Also suitable are nucleic acids having morpholino backbone structures as described in, e.g., U.S. Pat. No. 5,034,506. For example, in some cases, a labeled detector RNA comprises a 6-membered morpholino ring in place of a ribose ring. In some cases, a phosphorodiamidate or other non-phosphodiester internucleoside linkage replaces a phosphodiester linkage.

Suitable modified polynucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

### Mimetics

A labeled detector RNA can be a nucleic acid mimetic. The term “mimetic” as it is applied to polynucleotides is intended to include polynucleotides wherein only the furanose ring or both the furanose ring and the internucleotide linkage are replaced with non-furanose groups, replacement of only the furanose ring is also referred to in the art as being a sugar surrogate. The heterocyclic base moiety or a modified heterocyclic base moiety is maintained for hybridization with an appropriate target nucleic acid. One such nucleic acid, a polynucleotide mimetic that has been shown to have excellent hybridization properties, is referred to as a peptide nucleic acid (PNA). In PNA, the sugar-backbone of a polynucleotide is replaced with an amide containing backbone, in particular an aminoethylglycine backbone. The nucleotides are retained and are bound directly or indirectly to aza nitrogen atoms of the amide portion of the backbone.

One polynucleotide mimetic that has been reported to have excellent hybridization properties is a peptide nucleic acid (PNA). The backbone in PNA compounds is two or more linked aminoethylglycine units which gives PNA an amide containing backbone. The heterocyclic base moieties are bound directly or indirectly to aza nitrogen atoms of the amide portion of the backbone. Representative U.S. patents that describe the preparation of PNA compounds include, but are not limited to: U.S. Pat. Nos. 5,539,082; 5,714,331; and 5,719,262.

Another class of polynucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups has been selected to give a non-ionic oligomeric compound.

The non-ionic morpholino-based oligomeric compounds are less likely to have undesired interactions with cellular proteins. Morpholino-based polynucleotides are non-ionic mimics of oligonucleotides which are less likely to form undesired interactions with cellular proteins (Dwaine A. Braasch and David R. Corey, Biochemistry, 2002, 41 (14), 4503-4510). Morpholino-based polynucleotides are disclosed in U.S. Pat. No. 5,034,506. A variety of compounds within the morpholino class of polynucleotides have been prepared, having a variety of different linking groups joining the monomeric subunits.

A further class of polynucleotide mimetic is referred to as cyclohexenyl nucleic acids (CeNA). The furanose ring normally present in a DNA/RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation.

A further modification includes Locked Nucleic Acids (LNAs) in which the 2′-hydroxyl group is linked to the 4′ carbon atom of the sugar ring thereby forming a 2′-C,4′-C-oxymethylene linkage thereby forming a bicyclic sugar moiety. The linkage can be a methylene (—CH₂—), group bridging the 2′ oxygen atom and the 4′ carbon atom wherein n is 1 or 2 (Singh et al., Chem. Commun., 1998, 4, 455-456). LNA and LNA analogs display very high duplex thermal stabilities with complementary DNA and RNA (Tm=+3 to +10° C.), stability towards 3′-exonucleolytic degradation and good solubility properties. Potent and nontoxic antisense oligonucleotides containing LNAs have been described (Wahlestedt et al., Proc. Natl. Acad. Sci. U.S.A., 2000, 97, 5633-5638).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

### Modified Sugar Moieties

A labeled detector RNA can also include one or more substituted sugar moieties. Suitable polynucleotides comprise a sugar substituent group selected from: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C.sub.1 to C₁₀ alkyl or C₂ to C₁₀ alkenyl and alkynyl. Particularly suitable are O((CH₂)—O)ₘCH₃, O(CH₂), OCH₃, O(CH₂), NH₂, O(CH₂),CH₃, O(CH₂), ONH₂, and O(CH₂), ON((CH₂),CH₃)₂, where n and m are from 1 to about 10. Other suitable polynucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, CI, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A suitable modification includes 2′-methoxyethoxy (2′—O—CH₂ CH₂OCH₃, also known as 2′-O-(2-methoxyethyl) or 2′-MOE) (Martin et al., Helv. Chim. Acta, 1995, 78, 486-504) i.e., an alkoxyalkoxy group. A further suitable modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′—O—CH₂—O—CH₂—N(CH₃) 2.

Other suitable sugar substituent groups include methoxy (—O—CH₃), aminopropoxy (—O CH₂ CH₂ CH₂NH₂), allyl (—CH₂—CH═CH₂), —O-allyl (—O—CH₂—CH═CH₂) and fluoro (F). 2′-sugar substituent groups may be in the arabino (up) position or ribo (down) position. A suitable 2′-arabino modification is 2′-F. Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar.

### Base Modifications and Substitutions

A labeled detector RNA may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine (1H-pyrimido (5,4-b)(1,4)benzoxazin-2 (3H)-one), phenothiazine cytidine (1H-pyrimido (5,4-b)(1,4)benzothiazin-2 (3H)-one), G-clamps such as a substituted phenoxazine cytidine (e.g. 9-(2-aminoethoxy)-H-pyrimido (5,4-(b)(1,4)benzoxazin-2 (3H)-one), carbazole cytidine (2H-pyrimido (4,5-b) indol-2-one), pyridoindole cytidine (H-pyrido (3′,2′: 4,5) pyrrolo (2,3-d)pyrimidin-2-one).

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are useful for increasing the binding affinity of an oligomeric compound. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi et al., eds., Antisense Research and Applications, CRC Press, Boca Raton, 1993, pp. 276-278) and are suitable base substitutions, e.g., when combined with 2′-O-methoxyethyl sugar modifications.

### Devices

The present disclosure provides a device comprising: i) a Cas13Z polypeptide of the present disclosure; ii) a Cas13Z guide nucleic acid; and iii) a detector nucleic acid (also referred to herein as a “reporter” or a “reporter nucleic acid”). In some cases, the device comprises a lateral flow strip. In some cases, the reporter nucleic acid is immobilized on a solid support. In some cases, the device is configured to be coupled to a spectrophotometer. Examples of suitable devices are described in, e.g., US 2022/0099662 and US 2022/0090178. In some cases, devices as disclosed herein may be used for multiplexing, i.e., may be used to detect multiple (from 2 to 10, from 10 to 25, etc.)distinct target nucleic acids in a sample.

In some cases, a subject device comprises: i) a first chamber comprising a sample (e.g., a sample comprising cells) and a buffer for lysing the sample (e.g., for lysing cells in a sample); ii) a second chamber, fluidically connected by a first pneumatic valve to the first chamber, where the second chamber comprises a Cas13Z polypeptide and a reporter comprising a nucleic acid and a detection moiety (a “reporter nucleic acid”; also referred to as “a reporter”; also referred to as a “detector nucleic acid” where detector nucleic acids are described above), and where the second chamber is coupled to a measurement device for measuring the signal from the detection moiety produced by cleavage of the nucleic acid of the reporter. In some cases, the device further comprises (iii) a third chamber fluidically connected by the first pneumatic valve to the first chamber and connected by a second pneumatic valve to the second chamber. In some cases, the first pneumatic valve fluidically connecting the first chamber and the second chamber comprises a first channel adjacent to a first microfluidic channel connecting the first chamber and the second chamber. In some cases, the first pneumatic valve fluidically connecting the first chamber and the third chamber comprises a second channel adjacent to a second microfluidic channel connecting the first chamber and the third chamber. In some cases, the second pneumatic valve fluidically connecting the second chamber and the third chamber comprises a third channel adjacent to a third microfluidic channel connecting the second chamber and the third chamber. In some cases, the first channel, the second channel, or the third channels are connected to an air manifold. In some cases, more than one chamber comprising a Cas13Z polypeptide and a reporter nucleic acid are fluidically connected to a single chamber comprising the sample. In some cases, more than one chamber comprising a Cas13Z polypeptide and a reporter nucleic acid are fluidically connected to a single chamber comprising a forward primer, a reverse primer, a dNTP, and a polymerase.

In various aspects, the present disclosure provides a device for measuring a signal, where the device comprises: a sliding layer comprising a channel with an opening at a first end of the channel and an opening at a second end of the channel; and a fixed layer comprising: i) a first chamber having an opening; ii) a second chamber having an opening, wherein the second chamber comprises a Cas13Z polypeptide and a reporter nucleic acid (a nucleic acid comprising a detection moiety; as described above); iii) a first side channel having an opening aligned with the opening of the first chamber; and iv) a second side channel having an opening aligned with the opening of the second chamber, wherein the sliding layer and the fixed layer move relative to each other to fluidically connect the first chamber and the first side channel via the opening at the first end of the channel, the opening at the second end of the channel, the opening of the first chamber, and the opening of the first side channel, and wherein the sliding layer and the fixed layer move relative to each other to fluidically connect the second chamber and the second side channel via the opening at the first end of the channel, the opening at the second end of the channel, the opening of the second chamber, and the opening of the second side channel.

In some cases, the fixed layer further comprises i) a third chamber having an opening; and ii) a third side channel having an opening aligned with the opening of the third chamber, wherein the sliding layer and the fixed layer move relative to each other to fluidically connect the third chamber and the third side channel via the opening at the first end of the channel, the opening at the second end of the channel, the opening of the third chamber, and the opening of the third side channel. In some cases, the second chamber is coupled to a measurement device for measuring the signal from the detection moiety produced by cleavage of the nucleic acid of the reporter nucleic acid. In some cases, the opening of the first end of the channel overlaps with the opening of the first chamber and the opening of the second end of the channel overlaps with the opening of the first side channel.

In some cases, the opening of the first end of the channel overlaps with the opening of the second chamber and the opening of the second end of the channel overlaps with the opening of the second side channel. In some cases, the opening of the first end of the channel overlaps with the opening of the third chamber and the opening of the second end of the channel overlaps with the opening of the third channel. In some cases, the first side channel, the second side channel, and the third side channel are fluidically connected to a mixing chamber.

In some cases, the third chamber comprises one or more reagents for amplification of a nucleic acid. For example, in some case, the third chamber comprises a forward primer, a reverse primer, a dNTP, an NTP, a polymerase, a reverse transcriptase, a T7 polymerase, or any combination thereof. In some cases, the forward primer, the reverse primer, or both comprises a T7 promoter. In some cases, the second chamber comprises a guide nucleic acid. In some cases, the Cas13Z polypeptide, the reporter nucleic acid, the guide nucleic acid, the forward primer, the reverse primer, the dNTP, the NTP, the polymerase, the reverse transcriptase, the T7 promoter, the T7 polymerase, or any combination thereof is lyophilized or vitrified.

In some cases, the second chamber is optically connected to a spectrophotometric measurement device or a fluorescence measurement device. In some cases, the second chamber comprises a metal lead adapted for measurement of a change in current. In some cases, the first chamber holds a volume of about 200 μL, the second chamber holds a volume of about 20 μL, and the third chamber holds a volume of about 20 μL. In some cases, the second chamber comprises a plurality of guide RNAs.

In some cases, the device comprises from 2 to 20 chambers comprising a Cas13Z polypeptide, a guide nucleic acid, and a reporter nucleic acid, wherein a detection chamber of the from 2 to 20 chambers comprises a unique guide nucleic acid. In some cases, the reporter nucleic acid is a hybrid reporter having at least one ribonucleotide and at least one deoxyribonucleotide. In some cases, the reporter nucleic acid is immobilized on a surface. In some cases, the surface is a surface of the first chamber or a surface of a bead.

In various aspects, the present disclosure provides a device comprising: a chamber comprising i) a Cas13Z polypeptide; and ii) an immobilized reporter nucleic acid comprising a nucleic acid, an affinity molecule (e.g., biotin), and a detection moiety; and a lateral flow strip comprising: i) a first region comprising a capture molecule specific for the affinity molecule; and ii) a second region comprising an antibody, wherein the first region is upstream of the second region and the chamber is upstream of the lateral flow strip and wherein the first molecule binds to the second molecule.

In some cases, the first molecule is conjugated to a 3′ end or a 5′ end of the nucleic acid of the reporter nucleic acid, and wherein the first molecule is directly conjugated to the detection moiety. In some cases, the detection moiety comprises a fluorophore. In some cases, the antibody on the second region is specific for an antibody-coated nanoparticle. In some cases, the antibody-coated nanoparticle binds the fluorophore. In some cases, the chamber further comprises a second immobilized reporter (a second immobilized reporter nucleic acid) comprising a second nucleic acid, a second detection moiety, and the first molecule. In some cases, the first molecule is conjugated to a 3′ end or a 5′ end of the second nucleic acid, and wherein the first molecule is directly conjugated to the second detection moiety. In some cases, the lateral flow strip comprises a third region comprising a second antibody. In some cases, the antibody binds the fluorophore and the second antibody binds the second fluorophore. In some cases, the immobilized reporter, the second immobilized reporter, or both are conjugated to a magnetic bead. In some cases, the chamber interfaces with a magnet. In some cases, the device is connected to a sample prep device comprising a sample chamber, upstream, of an amplification chamber, upstream of the chamber. In some cases, the sample chamber, the amplification chamber, the reaction chamber, and the lateral flow strip are separated by a substrate. In some cases, each chamber of the sample prep device comprises a notch preventing fluid flow. In some cases, the sample prep device comprises a rotatable element and wherein the rotatable element controls fluid flow between chambers.

In various aspects, the present disclosure provides a method of detecting a presence or an absence of a target nucleic acid in a sample, the method comprising: contacting a first volume to a second volume, where the first volume comprises the sample and the second volume comprises: i) a Cas13Z guide nucleic acid having at least 10 nucleotides reverse complementary to a target nucleic acid in the sample; and ii) a Cas13Z polypeptide activated upon binding of the guide nucleic acid to the target nucleic acid; iii) a reporter nucleic acid (“detector nucleic acid”) comprising a nucleic acid and a detection moiety, where the second volume is at least 4-fold greater than the first volume; and detecting the presence or the absence of the target nucleic acid by measuring a signal produced by cleavage of the nucleic acid of the reporter, where cleavage occurs when the Cas13Z polypeptide is activated.

In some instances, the device comprises: (a) a chamber comprising i) a Cas13Z polypeptide; and ii) an immobilized reporter nucleic acid comprising a nucleic acid comprising a detection moiety (e.g., a fluorophore), and an affinity molecule conjugated to the detection moiety; and (b) a lateral flow strip comprising: i) a first region comprising a capture molecule (e.g., an antibody) specific for the affinity molecule; and ii) a second region comprising an antibody, wherein the first region is upstream of the second region and the chamber is upstream of the lateral flow strip and wherein the affinity molecule binds to the capture molecule.

The device can be used in a detection method of the present disclosure (i.e., a method of detecting a target RNA). Thus, the present disclosure provides any of the above devices for use in a method of detecting a presence of an absence of a target nucleic acid in a sample, the method comprising: contacting a first volume to a second volume, wherein the first volume comprises the sample and the second volume comprises: i) a guide nucleic acid having at least 10 nucleotides reverse complementary to a target nucleic acid in the sample; and ii) a Cas13Z polypeptide activated upon binding of the guide nucleic acid to the target nucleic acid; iii) a reporter comprising a nucleic acid and a detection moiety, wherein the second volume is at least 4-fold greater than the first volume; and detecting the presence or the absence of the target nucleic acid by measuring a signal produced by cleavage of the nucleic acid of the reporter, wherein cleavage occurs when the Cas13Z polypeptide is activated.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
Aspect 1. A composition comprising: a) a Cas13Z polypeptide comprising an amino acid sequence having at least 50% amino acid sequence identity to the amino acid sequence depicted in any one of FIG. 1-5; and b) a Cas13Z guide RNA comprising: i) an activation region that binds to the Cas13Z polypeptide; and ii) a targeting region that comprises a nucleotide sequence that is complementary to a target sequence of a target RNA, wherein the activation region is heterologous to the targeting region, and optionally wherein the targeting region is not 100% complementary to a bacterial nucleic acid, a viral nucleic acid, an archaeal nucleic acid, or a bacteriophage nucleic acid.
Aspect 2. The composition of aspect 1, wherein the Cas13Z polypeptide comprises an amino acid sequence having at least 75% amino acid sequence identity to the amino acid sequence depicted in any one of FIG. 1-5.
Aspect 3, The composition of aspect 1, wherein the Cas13Z polypeptide comprises an amino acid sequence having at least 95% amino acid sequence identity to the amino acid sequence depicted in any one of FIG. 1-5.
Aspect 4. The composition of any one of aspects 1-3, wherein the nucleotide sequence that is complementary to a target sequence of a target RNA is 15 nucleotides to 18 nucleotides long.
Aspect 5. The composition of any one of aspects 1-3, wherein the nucleotide sequence that is complementary to a target sequence of a target RNA is 18 nucleotides to 25 nucleotides long.
Aspect 6. The composition of any one of aspects 1-5, wherein the composition comprises a lipid.
Aspect 7. The composition of any one of aspects 1-6, wherein a) and b) are within a liposome.
Aspect 8. The composition of any one of aspects 1-6, wherein a) and b) are within a particle.
Aspect 9. The composition of any one of aspects 1-8, comprising one or more of: a buffering agent, a nuclease inhibitor, a detergent, a polyaminc, a stabilizing agent, and a protease inhibitor.
Aspect 10. The composition of any one of aspects 1-9, wherein the region that hybridizes to a target RNA hybridizes to a eukaryotic target RNA.
Aspect 11. The composition of any one of aspects 1-10, wherein the Cas 13Z polypeptide comprises a first higher eukaryotes and prokaryotes nucleotide-binding (HEPN) domain and a second HEPN domain.
Aspect 12. The composition of any one of aspects 1-11, wherein the Cas13Z polypeptide comprises a mutation in the first HEPN domain and/or the second HEPN domain.
Aspect 13. The composition of aspect 12, wherein the catalytic activity of the Cas13Z is reduced compared to the catalytic activity of a Cas13Z polypeptide comprising an amino acid sequence depicted in one of FIG. 1-5.
Aspect 14. The composition of any one of aspects 1-13, wherein the composition is lyophilized.
Aspect 15. The composition of any one of aspects 1-14, wherein the Cas13Z guide RNA comprise one or more heterologous moieties.
Aspect 16. The composition of aspect 15, wherein said one or more heterologous moieties is one or more polyamines, one or more polyamides, one or more polyethylene glycols, one or more polyethers, one or more cholesterol moieties, one or more cholic acids, one or more thioesters, one or more thiocholesterols, one or more lipids, one or more aliphatic chains, one or more phospholipids, one or more adamantane acetic acids, one or more palmityl moieties, one or more octadecylamine or hexylamino-carbonyl-oxycholesterol moieties, one or more biotins, one or more phenazines, one or more folates, one or more phenanthridines, one or more anthraquinones, one or more acridines, one or more fluoresceins, one or more rhodamines, one or more coumarins, one or more dyes, or any combination thereof.
Aspect 17. The composition of any one of aspects 1-16, wherein the Cas 13Z guide RNA comprises one or more modified sugar moieties, one or more modified nucleobases, one or more nucleic acid mimetics, one or more non-natural internucleoside linkages, which are one or more phosphorothioates, one or more inverted polarity linkages, one or more abasic nucleoside linkages, or any combination thereof.
Aspect 18. The composition of aspect 17, wherein the non-natural internucleoside linkage comprises a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a, a 3′-amino phosphoramidate, an aminoalkylphosphoramidate, a phosphorodiamidate, a thionophosphoramidate, a thionoalkylphosphonate, a thionoalkylphosphotriester, a selenophosphate, or a boranophosphate.
Aspect 19. The composition of aspect 17, wherein said one or more modified sugar moieties are one or more locked nucleic acid (LNA) sugar moieties, one or more 2′-substituted sugar moieties, one or more 2′-O-methoxyethyl modified sugar moieties, one or more 2′-O-methyl modified sugar moieties, one or more 21-O-(2-methoxyethyl) modified sugar moieties, one or more 2′-fluoro modified sugar moieties, one or more 2′-dimethylaminooxyethoxy modified sugar moieties, one or more 2′-dimethylaminoethoxyethoxy modified sugar moieties, or any combination thereof.
Aspect 20. The composition of aspect 17, wherein said one or more nucleic acid mimetics are one or more peptide nucleic acids (PNAs), one or more morpholino nucleic acids, one or more cyclohexenyl nucleic acids (CeNAs), or any combination thereof.
Aspect 21. The composition of aspect 17, wherein said one or more modified nucleobases are one or more 5-methylcytosines; one or more 5-hydroxymethyl cytosines; one or more xanthines; one or more hypoxanthines; one or more 2-aminoadenines; one or more 6-methyl derivatives of adenine; one or more 6-methyl derivatives of guanine; one or more 2-propyl derivatives of adenine; one or more 2-propyl derivatives of guanine; one or more 2-thiouracils; one or more 2-thiothymines; one or more 2-thiocytosines; one or more 5-propynyl uracils; one or more 5-propynyl cytosines; one or more 6-azo uracils; one or more 6-azo cytosines; one or more 6-azo thymines; one or more pseudouracils; one or more 4-thiouracils; an 8-haloadenins; one or more 8-aminoadenines; one or more 8-thioladeninse; one or more 8-thioalkyladenines; one or more 8-hydroxyladenines; one or more 8-haloguanines; one or more 8-aminoguanines; one or more 8-thiolguanines; one or more 8-thioalkylguanines; one or more 8-hydroxylguanines; one or more 5-halouracils; one or more 5-bromouracils; one or more 5-trifluoromethyluracils; one or more 5-halocytosines; one or more 5-bromocytosines; one or more 5-trifluoromethylcytosines; one or more 5-substituted uracils; one or more 5-substituted cytosines; one or more 7-methylguanines; one or more 7-methyladenines; one or more 2-F-adenines; one or more 2-amino-adenines; one or more 8-azaguanines; one or more 8-azaadenines; one or more 7-deazaguanines; one or more 7-deazaadenines; one or more 3-deazaguanines; one or more 3-deazaadenines; one or more tricyclic pyrimidines; one or more phenoxazine cytidines; one or more phenothiazine cytidines; one or more substituted phenoxazine cytidines; one or more carbazole cytidines; one or more pyridoindole cytidines; one or more 7-deazaguanosincs; one or more 2-aminopyridines; one or more 2-pyridones; onc or more 5-substituted pyrimidines; one or more 6-azapyrimidines; one or more N-2, N-6 or 0-6 substituted purines; one or more 2-aminopropyladenines; one or more 5-propynyluracils; one or more 5-propynylcytosines, or any combination thereof.
Aspect 22. The composition of any one of aspects 1-21, wherein the Cas13Z polypeptide is fused to one or more heterologous polypeptides.
Aspect 23. A Cas13Z fusion polypeptide comprising: a) a Cas13Z polypeptide comprising an amino acid sequence having at least 50% amino acid sequence identity to the amino acid sequence depicted in any one of FIG. 1-5; and b) one or more heterologous polypeptides.
Aspect 24. The Cas13Z fusion polypeptide of aspect 23, wherein at least one of the one or more heterologous polypeptide exhibits an enzymatic activity that modifies a target RNA.
Aspect 25. The Cas13Z fusion polypeptide of aspect 24, wherein the enzymatic activity comprises methylase activity, demethylase activity, or deaminase activity.
Aspect 26. The Cas13Z fusion polypeptide of aspect 23, wherein at least one of the one or more heterologous polypeptide exhibits an enzymatic activity that modifies a target polypeptide associated with a target RNA.
Aspect 27. The Cas13Z fusion polypeptide of any one of aspects 23-26, wherein at least one of the one or more heterologous polypeptide facilitates entry of the Cas 13Z polypeptide into a eukaryotic cell.
Aspect 28. The Cas13Z fusion polypeptide of any one of aspects 23-27, wherein at least one of the one or more heterologous polypeptide is a nuclear export signal.
Aspect 29. The Cas13Z fusion polypeptide of any one of aspects 23-27, wherein at least one of the one or more heterologous polypeptide is a nuclear localization signal.
Aspect 30. A nucleic acid comprising a nucleotide sequence encoding the Cas13Z fusion polypeptide of any one of aspects 23-29.
Aspect 31. The nucleic acid of aspect 30, wherein the nucleotide sequence encoding the Cas13Z fusion polypeptide is operably linked to a promoter.
Aspect 32. The nucleic acid of aspect 31, wherein the promoter is a regulatable promoter.
Aspect 33. The nucleic acid of aspect 31 or aspect 32, wherein the promoter is functional in a eukaryotic cell.
Aspect 34. The nucleic acid of aspect 33, wherein the promoter is functional in one or more of: a plant cell, a fungal cell, an animal cell, cell of an invertebrate, an insect cell, an arthropod cell, an arachnid cell, a cell of a vertebrate, a mammalian cell, a primate cell, a non-human primate cell, and a human cell.
Aspect 35. The nucleic acid of aspect 33 or aspect 34, wherein the promoter is a cell type-specific promoter or a tissue-specific promoter.
Aspect 36. A recombinant expression vector comprising the nucleic acid of any one of aspects 30-35.
Aspect 37. The recombinant expression vector of aspect 36, wherein the recombinant expression vector is a recombinant adenoassociated viral vector, a recombinant retroviral vector, or a recombinant lentiviral vector.
Aspect 38. A recombinant expression vector comprising one or more nucleotide sequences encoding: a) a Cas13Z polypeptide comprising an amino acid sequence having at least 50% amino acid sequence identity to the amino acid sequence depicted in any one of FIG. 1-5; and b) a Cas13Z guide RNA comprising: i) an activation region that binds to the Cas13Z polypeptide; and ii) a targeting region that comprises a nucleotide sequence that is complementary to a target sequence of a target RNA.
Aspect 39. A eukaryotic cell comprising one or more of: a) a Cas13Z polypeptide comprising an amino acid sequence having at least 50% amino acid sequence identity to the amino acid sequence depicted in any one of FIG. 1-5; b) a nucleic acid molecule encoding a Cas13Z polypeptide comprising an amino acid sequence having at least 50% amino acid sequence identity to the amino acid sequence depicted in any one of FIG. 1-5; c) a Cas13Z fusion polypeptide comprising: i) a Cas13Z polypeptide comprising an amino acid sequence having at least 50% amino acid sequence identity to the amino acid sequence depicted in any one of FIG. 1-5; and ii) one or more heterologous polypeptides; d) a nucleic acid comprising a nucleotide sequence encoding a Cas13Z fusion polypeptide, and e) a Cas13Z guide RNA comprising: i) an activation region that binds to the Cas13Z polypeptide; and ii) a targeting region that comprises a nucleotide sequence that is complementary to a target sequence of a target RNA; and f) a nucleic acid comprising a nucleotide sequence encoding a Cas 13Z guide RNA.
Aspect 40. The eukaryotic cell of aspect 39, wherein the eukaryotic cell is a plant cell, a mammalian cell, an insect cell, an arachnid cell, a fungal cell, a bird cell, a reptile cell, an amphibian cell, an invertebrate cell, a mouse cell, a rat cell, a primate cell, a non-human primate cell, or a human cell.
Aspect 41. A method of editing a target RNA, the method comprising contacting a target RNA with: a) a Cas13Z polypeptide comprising an amino acid sequence having at least 50% amino acid sequence identity to the amino acid sequence depicted in any one of FIG. 1-5; and b) a Cas13Z guide RNA comprising: i) an activation region that binds to the Cas13Z polypeptide; and ii) a targeting region that comprises a nucleotide sequence that is complementary to a target sequence of the target RNA.
Aspect 42. The method of aspect 41, wherein the method comprises deaminating one or more nucleotides of a target RNA.
Aspect 43. The method of aspect 41, wherein the method comprises methylating one or more nucleotides of a target RNA.
Aspect 44. The method of aspect 41, wherein the method comprises demethylating one or more nucleotides of a target RNA.
Aspect 45. A method of detecting a target RNA in a sample comprising a plurality of RNAs that differ from one another in nucleotide sequence, the method comprising: a) contacting the sample with: i) a Cas13Z polypeptide comprising an amino acid sequence having at least 50% amino acid sequence identity to the amino acid sequence depicted in any one of FIG. 1-5; ii) a Cas13Z guide RNA comprising a region that hybridizes with the target RNA; and iii) a labeled detector RNA that does not comprise a region that hybridizes with the target RNA; b) detecting a signal produced by cleavage of the labeled detector RNA by the Cas13Z polypeptide.
Aspect 46. The method of aspect 45, wherein the target RNA in the sample is present in a range of from 50 fM to 1 nM.
Aspect 47. The method of aspect 45, wherein the target RNA in the sample is present in a range of from 500 fM to 1 nM.
Aspect 48. The method of aspect 45, wherein the target RNA in the sample is present in a range of from 1 pM to 1 nM.
Aspect 49. The method of any one of aspects 45-48, wherein the plurality of RNAs comprise from 5 to 10⁷ RNAs that differ from one another in nucleotide sequence.
Aspect 50. The method of any one of aspects 45-49, wherein measuring a detectable signal comprises one or more of: gold nanoparticle-based detection, fluorescence polarization, colloid phase transition/dispersion, electrochemical detection, fluorescent signal detection, and semiconductor-based sensing.
Aspect 51. The method of any one of aspects 45-50, wherein the labeled detector RNA comprises a fluorescence-emitting dye pair.
Aspect 52. The method of any one of aspects 45-50, wherein the labeled detector RNA comprises a quencher/fluor pair.
Aspect 53. The method of any one of aspects 45-52, wherein the labeled detector RNA comprises one or more of: a non-natural internucleoside linkage, a nucleic acid mimetic, a modified sugar moiety, a modified nucleobase, a locked nucleic acid (LNA), a peptide nucleic acid (PNA), a morpholino nucleic acid, and a cyclohexenyl nucleic acid (CeNA).
Aspect 54. The method of any one of aspects 45-53, wherein the target RNA is from a virus, a parasite, a helminth, a fungus, a protozoan, a bacterium, or a pathogenic bacterium.
Aspect 55. The method of any one of aspects 45-53, wherein the target RNA is from a virus selected from: Zika virus, human immunodeficiency virus (HIV), hepatitis B virus, hepatitis C virus, herpes virus, herpes simplex virus I, herpes simplex virus II, papillomavirus, rabies virus, cytomegalovirus, human serum parvo-like virus, respiratory syncytial virus, varicella-zoster virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, west Nile virus, a coronavirus, and yellow fever virus.
Aspect 56. The method of any one of aspects 45-53, wherein the target RNA is from pathogenic bacteria selected from: Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Treponema pallidum, Lyme disease spirochetes, Pseudomonas aeruginosa, Mycobacterium leprae, and Brucella abortus.
Aspect 57. The method of any one of aspects 45-53, wherein the target RNA is from a human cell, an animal cell, a plant cell, a cancerous cell, an infected cell, or a diseased cell.
Aspect 58. The method of any one of aspects 45-53, wherein the target RNA is a transcript of a DNA molecule.
Aspect 59. A device comprising: i) a Cas13Z polypeptide comprising an amino acid sequence having at least 50% amino acid sequence identity to the amino acid sequence depicted in any one of FIG. 1-5; ii) a Cas13Z guide RNA comprising a region that hybridizes with the target RNA; and iii) a labeled detector RNA that does not comprise a region that hybridizes with the target RNA.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Example 1

Cas13Z protein was expressed in cell lysate in conjunction with a guide RNA (referred to as T guide RNA (“target guide RNA”)) designed to target the sfGFP sequence encoded on a target plasmid that was added to the mixture. The same reaction was set up to contain Cas13Z with a guide RNA that is incapable of hybridizing to any sequence on the target plasmid (referred to as NT guide RNA (“non-target guide RNA”)).

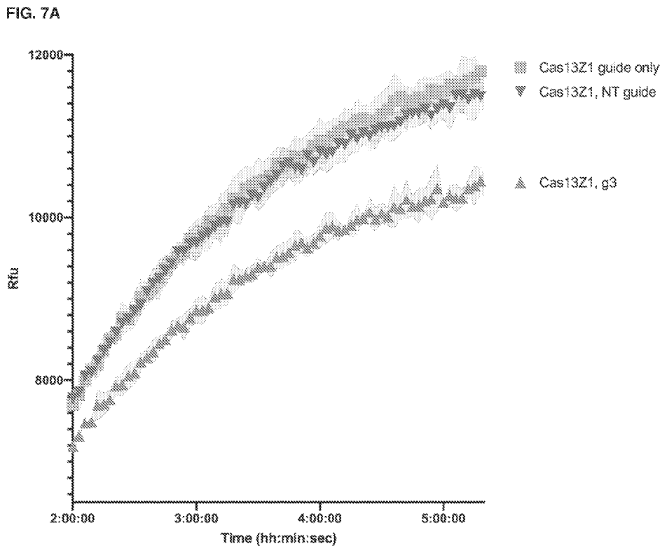

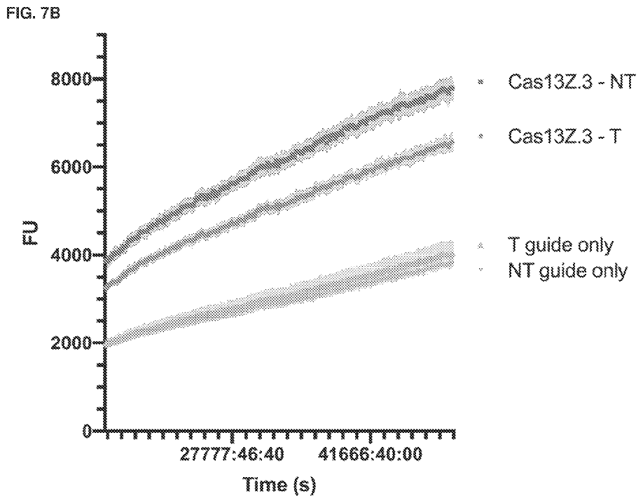

The data are shown in FIGS. 7A and 7B. Over a time course of 10 hours, Cas13Z expression with its T guide RNA leads to knockdown in GFP transcripts by proxy of GFP fluorescence intensity compared to Cas13Z incubated with the NT guide control. Addition of T guide only, without the Cas13Z protein, leads to minimal or no knockdown compared to the NT control.

### Example 2

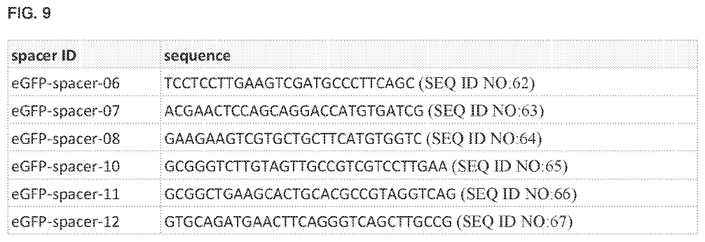

Cas13Z protein was expressed in mammalian cells (HEK293FT cells) in conjunction with a guide RNA containing a spacer sequence (referred to as eGFP-spacer and shown in FIG. 9) designed to target a GFP and/or BFP sequence, or a guide RNA that is incapable of hybridizing to a GFP and/or BFP sequence (referred to as NT guide (“non-target guide”)). HEK293FT cells were plated in a 96-well format at 15,000 cells per well with 100 μL of cell culture medium. Cells were transfected 24 hours later with 25 ng gRNA plasmid (U6 promoter-driven expression), 25 ng Cas 13z plasmid, 25 ng dox-inducible GFP plasmid, and 25 ng dox-inducible BFP plasmid using 0.32 μL lipofectamine and 10 μL of OptiMEM total. Media was changed 24 hours after and replaced with media containing 1 μg/mL doxycycline to induce GFP and BFP expression. Flow cytometry was performed 24 hours after induction (48 hours after transfection) on MACSQuant and Sartorius iQue3 flow cytometers.

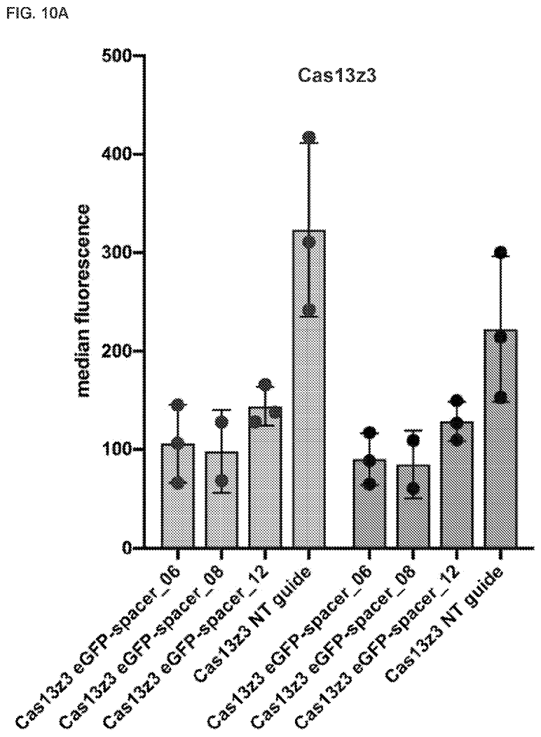

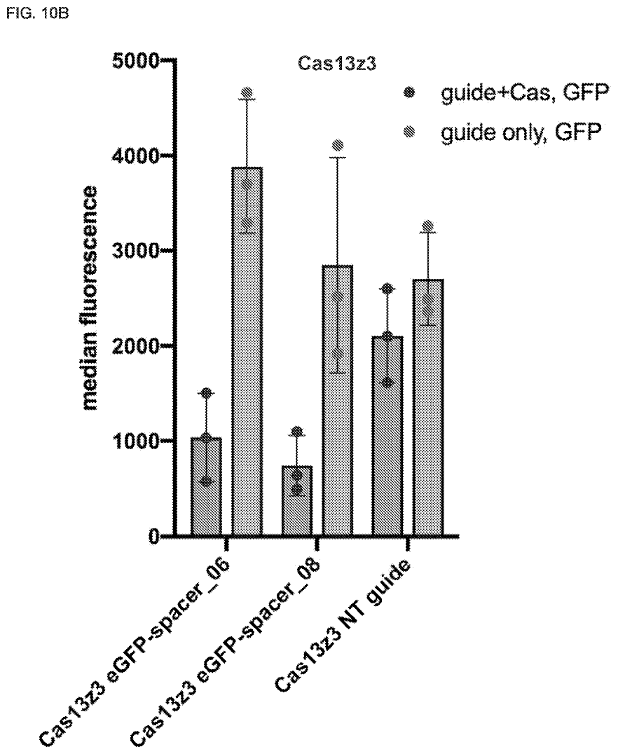

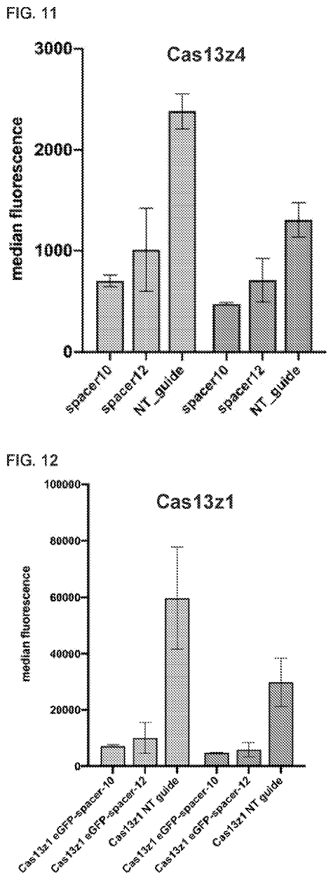

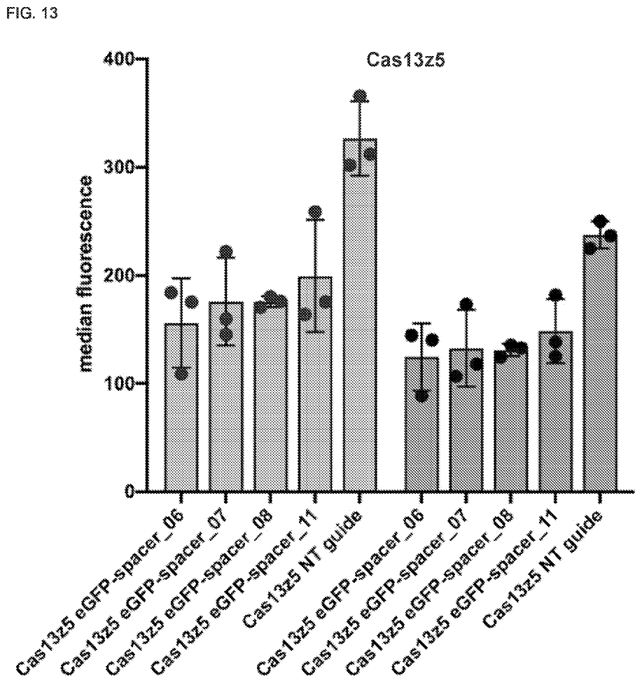

The data are shown in FIGS. 10-13. Expression of Cas13Z protein and a guide RNA containing an eGFP-spacer leads to knockdown in GFP and BFP transcripts by proxy of GFP and BFP fluorescence intensity compared to expression of Cas13Z protein with a NT guide control. The expression of a guide RNA containing an eGFP-spacer only, without expression of the Cas13Z protein, leads to minimal or no knockdown compared to the NT guide control.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A composition comprising:
a) a Cas 13Z polypeptide comprising an amino acid sequence having at least 50% amino acid sequence identity to the amino acid sequence depicted in any one of FIG. 1-5; and
b) a Cas13Z guide RNA comprising:
i) an activation region that binds to the Cas13Z polypeptide; and
ii) a targeting region that comprises a nucleotide sequence that is complementary to a target sequence of a target RNA,
wherein the activation region is heterologous to the targeting region, with the proviso that the targeting region is not 100% complementary to a naturally-occurring bacteriophage nucleic acid.

2. The composition of claim 1, wherein the Cas13Z polypeptide comprises an amino acid sequence having at least 75% amino acid sequence identity to the amino acid sequence depicted in any one of FIG. 1-5.

3. The composition of claim 1, wherein the Cas13Z polypeptide comprises an amino acid sequence having at least 95% amino acid sequence identity to the amino acid sequence depicted in any one of FIG. 1-5.

4. The composition of any one of claims 1-3, wherein the nucleotide sequence that is complementary to a target sequence of a target RNA is 15 nucleotides to 18 nucleotides long.

5. The composition of any one of claims 1-3, wherein the nucleotide sequence that is complementary to a target sequence of a target RNA is 18 nucleotides to 25 nucleotides long.

6. The composition of any one of claims 1-5, wherein the composition comprises a lipid.

7. The composition of any one of claims 1-6, wherein a) and b) are within a liposome.

8. The composition of any one of claims 1-6, wherein a) and b) are within a particle.

9. The composition of any one of claims 1-8, comprising one or more of: a buffering agent, a nuclease inhibitor, a detergent, a polyamine, a stabilizing agent, and a protease inhibitor.

10. The composition of any one of claims 1-9, wherein the region that hybridizes to a target RNA hybridizes to a eukaryotic target RNA.

11. The composition of any one of claims 1-10, wherein the Cas13Z polypeptide comprises a first higher eukaryotes and prokaryotes nucleotide-binding (HEPN) domain and a second HEPN domain.

12. The composition of any one of claims 1-11, wherein the Cas13Z polypeptide comprises a mutation in the first HEPN domain and/or the second HEPN domain.

13. The composition of claim 12, wherein the catalytic activity of the Cas13Z is reduced compared to the catalytic activity of a Cas13Z polypeptide comprising an amino acid sequence depicted in one of FIG. 1-5.

14. The composition of any one of claims 1-13, wherein the composition is lyophilized.

15. The composition of any one of claims 1-14, wherein the Cas13Z guide RNA comprise one or more heterologous moieties.

16. The composition of claim 15, wherein said one or more heterologous moieties is one or more polyamines, one or more polyamides, one or more polyethylene glycols, one or more polyethers, one or more cholesterol moieties, one or more cholic acids, one or more thioesters, one or more thiocholesterols, one or more lipids, one or more aliphatic chains, one or more phospholipids, one or more adamantane acetic acids, one or more palmityl moieties, one or more octadecylamine or hexylamino-carbonyl-oxycholesterol moieties, one or more biotins, one or more phenazines, one or more folates, one or more phenanthridines, one or more anthraquinones, one or more acridines, one or more fluoresceins, one or more rhodamines, one or more coumarins, one or more dyes, or any combination thereof.

17. The composition of any one of claims 1-16, wherein the Cas13Z guide RNA comprises one or more modified sugar moieties, one or more modified nucleobases, one or more nucleic acid mimetics, one or more non-natural internucleoside linkages, which are one or more phosphorothioates, one or more inverted polarity linkages, one or more abasic nucleoside linkages, or any combination thereof.

18. The composition of claim 17, wherein the non-natural internucleoside linkage comprises a phosphorothioate, a phosphoramidate, a non-phosphodiester, a heteroatom, a chiral phosphorothioate, a phosphorodithioate, a phosphotriester, an aminoalkylphosphotriester, a 3′-alkylene phosphonates, a 5′-alkylene phosphonate, a chiral phosphonate, a phosphinate, a, a 3′-amino phosphoramidate, an aminoalkylphosphoramidate, a phosphorodiamidate, a thionophosphoramidate, a thionoalkylphosphonate, a thionoalkylphosphotriester, a selenophosphate, or a boranophosphate.

19. The composition of claim 17, wherein said one or more modified sugar moieties are one or more locked nucleic acid (LNA) sugar moieties, one or more 2′-substituted sugar moieties, one or more 2′-O-methoxyethyl modified sugar moieties, one or more 2′-O-methyl modified sugar moieties, one or more 2′-O-(2-methoxyethyl) modified sugar moieties, one or more 2′-fluoro modified sugar moieties, one or more 2′-dimethylaminooxyethoxy modified sugar moieties, one or more 2′-dimethylaminoethoxyethoxy modified sugar moieties, or any combination thereof.

20. The composition of claim 17, wherein said one or more nucleic acid mimetics are one or more peptide nucleic acids (PNAs), one or more morpholino nucleic acids, one or more cyclohexenyl nucleic acids (CeNAs), or any combination thereof.

21. The composition of claim 17, wherein said one or more modified nucleobases are one or more 5-methylcytosines; one or more 5-hydroxymethyl cytosines; one or more xanthines; one or more hypoxanthines; one or more 2-aminoadenines; one or more 6-methyl derivatives of adenine; one or more 6-methyl derivatives of guanine; one or more 2-propyl derivatives of adenine; one or more 2-propyl derivatives of guaninc; one or more 2-thiouracils; one or more 2-thiothymines; one or more 2-thiocytosines; one or more 5-propynyl uracils; one or more 5-propynyl cytosines; one or more 6-azo uracils; one or more 6-azo cytosines; one or more 6-azo thymines; one or more pseudouracils; one or more 4-thiouracils; an 8-haloadenins; one or more 8-aminoadenines; one or more 8-thioladeninse; one or more 8-thioalkyladenines; one or more 8-hydroxyladenines; one or more 8-haloguanines; one or more 8-aminoguanines; one or more 8-thiolguanines; one or more 8-thioalkylguanines; one or more 8-hydroxylguanincs; one or more 5-halouracils; one or more 5-bromouracils; one or more 5-trifluoromethyluracils; one or more 5-halocytosines; one or more 5-bromocytosines; one or more 5-trifluoromethylcytosines; one or more 5-substituted uracils; one or more 5-substituted cytosines; one or more 7-methylguanines; one or more 7-methyladenines; one or more 2-F-adenines; one or more 2-amino-adenines; one or more 8-azaguanines; one or more 8-azaadenines; one or more 7-deazaguanines; one or more 7-deazaadenines; one or more 3-deazaguanines; one or more 3-deazaadenines; one or more tricyclic pyrimidines; one or more phenoxazine cytidines; one or more phenothiazine cytidines; one or more substituted phenoxazine cytidines; one or more carbazole cytidines; one or more pyridoindole cytidines; one or more 7-deazaguanosines; one or more 2-aminopyridines; one or more 2-pyridones; one or more 5-substituted pyrimidines; one or more 6-azapyrimidines; one or more N-2, N-6 or O-6 substituted purines; one or more 2-aminopropyladenines; one or more 5-propynyluracils; one or more 5-propynylcytosines, or any combination thereof.

22. The composition of any one of claims 1-21, wherein the Cas13Z polypeptide is fused to one or more heterologous polypeptides.

23. A Cas13Z fusion polypeptide comprising:
a) a Cas13Z polypeptide comprising an amino acid sequence having at least 50% amino acid sequence identity to the amino acid sequence depicted in any one of FIG. 1-5; and
b) one or more heterologous polypeptides.

24. The Cas13Z fusion polypeptide of claim 23, wherein at least one of the one or more heterologous polypeptide exhibits an enzymatic activity that modifies a target RNA.

25. The Cas13Z fusion polypeptide of claim 24, wherein the enzymatic activity comprises methylase activity, demethylase activity, or deaminase activity.

26. The Cas 13Z fusion polypeptide of claim 23, wherein at least one of the one or more heterologous polypeptide exhibits an enzymatic activity that modifies a target polypeptide associated with a target RNA.

27. The Cas13Z fusion polypeptide of any one of claims 23-26, wherein at least one of the one or more heterologous polypeptide facilitates entry of the Cas13Z polypeptide into a eukaryotic cell.

28. The Cas 13Z fusion polypeptide of any one of claims 23-27, wherein at least one of the one or more heterologous polypeptide is a nuclear export signal.

29. The Cas13Z fusion polypeptide of any one of claims 23-27, wherein at least one of the one or more heterologous polypeptide is a nuclear localization signal.

30. A nucleic acid comprising a nucleotide sequence encoding the Cas13Z fusion polypeptide of any one of claims 23-29.

31. The nucleic acid of claim 30, wherein the nucleotide sequence encoding the Cas13Z fusion polypeptide is operably linked to a promoter.

32. The nucleic acid of claim 31, wherein the promoter is a regulatable promoter.

33. The nucleic acid of claim 31 or claim 32, wherein the promoter is functional in a eukaryotic cell.

34. The nucleic acid of claim 33, wherein the promoter is functional in one or more of: a plant cell, a fungal cell, an animal cell, cell of an invertebrate, a fly cell, a cell of a vertebrate, a mammalian cell, a primate cell, a non-human primate cell, and a human cell.

35. The nucleic acid of claim 33 or claim 34, wherein the promoter is a cell type-specific promoter or a tissue-specific promoter.

36. A recombinant expression vector comprising the nucleic acid of any one of claims 30-35.

37. The recombinant expression vector of claim 36, wherein the recombinant expression vector is a recombinant adenoassociated viral vector, a recombinant retroviral vector, or a recombinant lentiviral vector.

38. A recombinant expression vector comprising one or more nucleotide sequences encoding:
a) a Cas13Z polypeptide comprising an amino acid sequence having at least 50% amino acid sequence identity to the amino acid sequence depicted in any one of FIG. 1-5; and
b) a Cas13Z guide RNA comprising:
i) an activation region that binds to the Cas13Z polypeptide; and
ii) a targeting region that comprises a nucleotide sequence that is complementary to a target sequence of a target RNA.

39. A eukaryotic cell comprising one or more of:
a) a Cas13Z polypeptide comprising an amino acid sequence having at least 50% amino acid sequence identity to the amino acid sequence depicted in any one of FIG. 1-5;
b) a nucleic acid molecule encoding a Cas13Z polypeptide comprising an amino acid sequence having at least 50% amino acid sequence identity to the amino acid sequence depicted in any one of FIG. 1-5;
c) a Cas13Z fusion polypeptide comprising: i) a Cas13Z polypeptide comprising an amino acid sequence having at least 50% amino acid sequence identity to the amino acid sequence depicted in any one of FIG. 1-5; and ii) one or more heterologous polypeptides;
d) a nucleic acid comprising a nucleotide sequence encoding a Cas13Z fusion polypeptide, and
e) a Cas 13Z guide RNA comprising: i) an activation region that binds to the Cas13Z polypeptide; and ii) a targeting region that comprises a nucleotide sequence that is complementary to a target sequence of a target RNA; and
f) a nucleic acid comprising a nucleotide sequence encoding a Cas13Z guide RNA.

40. The eukaryotic cell of claim 39, wherein the eukaryotic cell is a plant cell, a mammalian cell, an insect cell, an arachnid cell, a fungal cell, a bird cell, a reptile cell, an amphibian cell, an invertebrate cell, a mouse cell, a rat cell, a primate cell, a non-human primate cell, or a human cell.

41. A method of editing a target RNA, the method comprising contacting a target RNA with:
a) a Cas13Z polypeptide comprising an amino acid sequence having at least 50% amino acid sequence identity to the amino acid sequence depicted in any one of FIG. 1-5; and
b) a Cas13Z guide RNA comprising: i) an activation region that binds to the Cas 13Z polypeptide; and ii) a targeting region that comprises a nucleotide sequence that is complementary to a target sequence of the target RNA.

42. The method of claim 41, wherein the method comprises deaminating one or more nucleotides of a target RNA.

43. The method of claim 41, wherein the method comprises methylating one or more nucleotides of a target RNA.

44. The method of claim 41, wherein the method comprises demethylating one or more nucleotides of a target RNA.

45. A method of detecting a target RNA in a sample comprising a plurality of RNAs that differ from one another in nucleotide sequence, the method comprising:
a) contacting the sample with:
i) a Cas13Z polypeptide comprising an amino acid sequence having at least 50% amino acid sequence identity to the amino acid sequence depicted in any one of FIG. 1-5;
ii) a Cas13Z guide RNA comprising a region that hybridizes with the target RNA; and
iii) a labeled detector RNA that does not comprise a region that hybridizes with the target RNA;
b) detecting a signal produced by cleavage of the labeled detector RNA by the Cas13Z polypeptide.

46. The method of claim 45, wherein the target RNA in the sample is present in a range of from 50 fM to 1 nM.

47. The method of claim 45, wherein the target RNA in the sample is present in a range of from 500 fM to 1 nM.

48. The method of claim 45, wherein the target RNA in the sample is present in a range of from 1 pM to 1 nM.

49. The method of any one of claims 45-48, wherein the plurality of RNAs comprise from 5 to 10⁷ RNAs that differ from one another in nucleotide sequence.

50. The method of any one of claims 45-49, wherein measuring a detectable signal comprises one or more of: gold nanoparticle-based detection, fluorescence polarization, colloid phase transition/dispersion, electrochemical detection, fluorescent signal detection, and semiconductor-based sensing.

51. The method of any one of claims 45-50, wherein the labeled detector RNA comprises a fluorescence-emitting dye pair.

52. The method of any one of claims 45-50, wherein the labeled detector RNA comprises a quencher/fluor pair.

53. The method of any one of claims 45-52, wherein the labeled detector RNA comprises one or more of: a non-natural internucleoside linkage, a nucleic acid mimetic, a modified sugar moiety, a modified nucleobase, a locked nucleic acid (LNA), a peptide nucleic acid (PNA), a morpholino nucleic acid, and a cyclohexenyl nucleic acid (CeNA).

54. The method of any one of claims 45-53, wherein the target RNA is from a virus, a parasite, a helminth, a fungus, a protozoan, a bacterium, or a pathogenic bacterium.

55. The method of any one of claims 45-53, wherein the target RNA is from a virus selected from: Zika virus, human immunodeficiency virus (HIV), hepatitis B virus, hepatitis C virus, herpes virus, herpes simplex virus I, herpes simplex virus II, papillomavirus, rabies virus, cytomegalovirus, human serum parvo-like virus, respiratory syncytial virus, varicella-zoster virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, west Nile virus, a coronavirus, and yellow fever virus.

56. The method of any one of claims 45-53, wherein the target RNA is from pathogenic bacteria selected from: Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Treponema pallidum, Lyme disease spirochetes, Pseudomonas aeruginosa, Mycobacterium leprae, and Brucella abortus.

57. The method of any one of claims 45-53, wherein the target RNA is from a human cell, an animal cell, a plant cell, a cancerous cell, an infected cell, or a diseased cell.

58. The method of any one of claims 45-53, wherein the target RNA is a transcript of a DNA molecule.

59. A device comprising:
i) a Cas13Z polypeptide comprising an amino acid sequence having at least 50% amino acid sequence identity to the amino acid sequence depicted in any one of FIG. 1-5;
ii) a Cas13Z guide RNA comprising a region that hybridizes with the target RNA; and
iii) a labeled detector RNA that does not comprise a region that hybridizes with the target RNA.
